SEQUENCE LISTING (16 sequences)

SEQ ID NO: 1 - DNA, 19 bp, Artificial sequence
  Synthetic primer
catgctgtgaattgggctg

SEQ ID NO: 2 - DNA, 23 bp, Artificial sequence
  Synthetic primer
ccctctggtgatactttagcagt

SEQ ID NO: 3 - DNA, 16 bp, Artificial sequence
  Synthetic primer
gcatgctgcccggacc

SEQ ID NO: 4 - DNA, 18 bp, Artificial sequence
  Synthetic primer
acatgccaggtcgctggt

SEQ ID NO: 5 - DNA, 20 bp, Artificial sequence
  Synthetic primer
tccgcgacgtggagaatatc

SEQ ID NO: 6 - DNA, 20 bp, Artificial sequence
  Synthetic primer
tggtgtggtaaggacatcgc

SEQ ID NO: 7 - DNA, 20 bp, Artificial sequence
  Synthetic primer
tcaacatgttagccgcttgc

SEQ ID NO: 8 - DNA, 20 bp, Artificial sequence
  Synthetic primer
atggcaacctcaagtcctcc

SEQ ID NO: 9 - DNA, 20 bp, Artificial sequence
  Synthetic primer
ggaactacaggcaaccccaa

SEQ ID NO: 10 - DNA, 21 bp, Artificial sequence
  Synthetic primer
tcatctgggcaaggattgact

SEQ ID NO: 11 - DNA, 20 bp, Artificial sequence
  Synthetic primer
ttgaagagtggccggtttgt

SEQ ID NO: 12 - DNA, 20 bp, Artificial sequence
  Synthetic primer
tgcagaaaactggctccgat

SEQ ID NO: 13 - DNA, 20 bp, Artificial sequence
  Synthetic primer
agacgagcttgacttctcca

SEQ ID NO: 14 - DNA, 20 bp, Artificial sequence
  Synthetic primer
tgcattcggctcttcttcgt

SEQ ID NO: 15 - DNA, 22 bp, Artificial sequence
  Synthetic primer
tgccttgcttgagatagttcac

SEQ ID NO: 16 - DNA, 20 bp, Artificial sequence
  Synthetic primer
tcacttggaccccagtcaca

(19) United States
(12) Patent Application Publication
(10) Pub. No.: US 2022/0002291 A1
(43) Pub. Date: Jan. 6, 2022

(54) PROTEOLYSIS-TARGETING CHIMERAS

(71) Applicants: The Wistar Institute of Anatomy and Biology, Philadelphia, PA (US); Thomas Jefferson University, Philadelphia, PA (US)

(72) Inventors: Joseph SALVINO, Chester Springs, PA (US); Bruno CALABRETTA, Philadelphia, PA (US); Marco DE DOMINICI, Philadelphia, PA (US); You Cai XIAO, Philadelphia, PA (US)

(73) Assignees: The Wistar Institute of Anatomy and Biology, Philadelphia, PA (US); Thomas Jefferson University, Philadelphia, PA (US)

(21) Appl. No.: 17/290,789

(22) PCT Filed: Oct. 30, 2019
(86) PCT No.: PCT/US2019/058980

§ 371 Date: May 3, 2021

Related U.S. Application Data
(60) Provisional application No. 62/755,038, filed on Nov. 2, 2018.

(51) Int. Cl. C07D 471/04; A61K 45/06; A61K 31/519

(52) U.S. Cl. CPC C07D 471/04; A61K 31/519; A61K 45/06

(57) ABSTRACT

The present disclosure provides compounds of the formula (I) wherein these compounds contain a ligand which binds to one or more target proteins such as CDK4 or CDK6 and a ligand which binds to the machinery associated with the ubiquitinating protein machinery. Also provided herein are methods of using these compounds in compositions or methods of treating patients with these compounds for the treatment of a disease or disorders such as cancer.

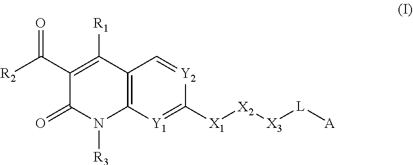

This application claims the benefit of priority from U.S. Provisional Patent Application No. 62/755,038, filed on Nov. 2, 2018, the entire contents of which are hereby incorporated by reference.

### BACKGROUND

### 1. Field

The present disclosure relates generally to the field of medicinal chemistry and medicine. More particularly, it concerns methods using small molecule ligands for selectively degrading target proteins such as proteins involved in disease such as cancer.

### 2. Description of Related Art

Proteolysis-targeting chimeras (PROTACs) are bifunctional molecules comprised of two small molecule ligands, one with high affinity towards the target protein of interest, and the second for recruitment of an E3 ligase that ubiquitinates the protein and targets it for proteolysis by the 26S proteasome (Lai and Crews, 2017). The two ligands are joined by a flexible tether providing a highly modular approach to generate molecules designed to degrade and silence proteins through a mechanism differing from standard small molecule or antibody inhibition. This modular approach provides room to optimize for ligand affinity without concern for functional activity since silencing the protein relies on recruitment of an E3 ligase in close proximity to the protein for ubiquitination, not functional inhibition. Optimal length and hydrophobicity of the tether is important and must be empirically evaluated because if the tether is too short there may be significant steric interactions in the recruitment of the E3 ligase. Hydrophobicity of the tether should also be optimized.

Additionally, one must also consider recruitment of various E3 ubiquitin ligases and the tether length and hydrophobicity. There are three classes of E3 ligases that have been identified, which include the HECT, RING, and U-Box domain types. The HECT domain family members directly catalyze the final attachment of ubiquitin to their substrate protein, while RING and U-Box E3s do not have a direct catalytic role in protein ubiquitination (Robinson and Ardley, 2004 and Metzger et al., 2012). The Cullin-RING ligases are the most abundant. Small molecules targeting these enzymes provide a framework to optimize ligase-recruiting molecules (Bulatov et al., 2015). PROTACs show relatively specific target degradation and less off-target degradation than initially suggested by the ligand specificity because the E3 ligase recruited can affect the specificity of the PROTAC (Lai and Crews, 2017).

Therefore, there remains a need to develop new PROTACs which have enhanced linker length and hydrophobicity.

### SUMMARY

In one aspect, the present disclosure provides compounds of the formula:

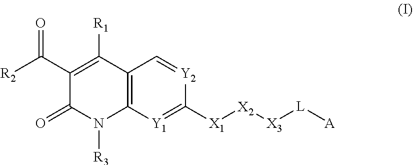

wherein:
R₁ is alkyl_{(C≤12)}, cycloalkyl_{(C≤12)}, aryl_{(C≤12)}, or a substituted version of any of these groups;
R₂ is alkyl_{(C≤12)}, cycloalkyl_{(C≤12)}, or a substituted version of either of these groups;
R₃ is cycloalkyl_{(C≤12)}, aryl_{(C≤12)}, or a substituted version of either of these groups;
Y₁ and Y₂ are each independently N or CH;
X₁ is O, S, or NRₐ,
   Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};

L is a linking group of the formula:

—(C(O))_{d}(CH₂)ₐX₄—(CH₂)_{b}—Y₃—X₅—(CH₂)_{c}— (IA)
wherein:
   d is 0 or 1;
   a, b, or c is 0, 1, 2, 3, 4, 5, or 6;
   X₄ is —C(O)—, —NR_{b}—, heteroarenediyl_{(C≤12)} or substituted heteroarenediyl_{(C≤12)};
      wherein R_{b} and R_{c} are each independently selected from hydrogen, alkyl_{(C≤6)}, or substituted alkyl_{(C≤6)};
   X₅ is —C(O)—, —NR_{b}—, —C(O)NR_{c}—, alkanediyl_{(C≤12)}, substituted alkanediyl_{(C≤12)}, arenediyl_{(C≤12)}, substituted arenediyl_{(C≤12)}, heteroarenediyl_{(C≤12)}, or substituted heteroarenediyl_{(C≤12)};
      wherein R_{b} and R_{c} are each independently selected from hydrogen, alkyl_{(C≤6)}, or substituted alkyl_{(C≤6)};
   Y₃ is a covalent bond, alkanediyl_{(C≤12)}, substituted alkanediyl_{(C≤12)}, —(CH₂CH₂O)ₑ(CH₂)_{f}—, —C(O)NR_{d}-alkanediyl_{(C≤12)}, or substituted —C(O)NR_{d}-alkanediyl_{(C≤12)};
   wherein:
      e is 1, 2, 3, 4, or 5;
      f is 0, 1, 2, 3, 4, or 5; and
      R_{d} is hydrogen, alkyl_{(C≤6)}, or substituted alkyl_{(C≤6)}; or
a linking group of the formula:

-(AA₁)ₓ- (IB)
wherein:
   AA₁ is an amino acid residue; and
   x is 1, 2, 3, 4, 5, or 6; and
A is hydrogen or an E3 ligase ligand; or
   a compound of the formula:

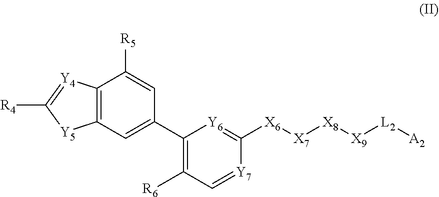

wherein:
R₄ is hydrogen, alkyl_{(C≤12)}, substituted alkyl_{(C≤12)}, cycloalkyl_{(C≤12)}, or substituted cycloalkyl_{(C≤12)};
R₅ and R₆ are each independently is hydrogen, halo, alkyl_{(C≤12)}, substituted alkyl_{(C≤12)}, cycloalkyl_{(C≤12)}, or substituted cycloalkyl_{(C≤12)};
Y₄, Y₆, and Y₇ are each independently N or CH;
Y₅ is O, S, or NR_{d}, wherein:
   R_{d} is hydrogen, alkyl_{(C≤12)}, substituted alkyl_{(C≤12)}, cycloalkyl_{(C≤12)}, or substituted cycloalkyl_{(C≤12)};
X₆ is O, S, or NRₑ,
   Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:

—(CH₂)_{g}X₁₀—(CH₂)ₕ—Y₈—X₁₁—(CH₂)ᵢ— (IIA)
wherein:
   g, h, and i are each independently 0, 1, 2, 3, 4, or 5;
   X₁₀ is —C(O)—, —NR_{f}—, heteroarenediyl_{(C≤12)} or substituted heteroarenediyl_{(C≤12)}, wherein:
      R_{f} is hydrogen, alkyl_{(C≤6)}, or substituted alkyl_{(C≤6)};
   X₁₁ is —C(O)—, —NR_{f}—, —C(O)NR_{g}—, heteroarenediyl_{(C≤12)} or substituted heteroarenediyl_{(C≤12)};
      wherein R_{f} and R_{g} are each independently selected from hydrogen, alkyl_{(C≤6)}, or substituted alkyl_{(C≤6)};
   Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
   wherein:
      j is 1, 2, 3, 4, or 5;
      k is 0, 1, 2, 3, 4, or 5; and
      R_{g} is hydrogen, alkyl_{(C≤6)}, or substituted alkyl_{(C≤6)}; or
a linking group of the formula:

-(AA₂)_{y}- (IIB)
wherein:
   AA₂ is an amino acid residue; and
   y is 1, 2, 3, 4, 5, or 6; and
A₂ is hydrogen or an E3 ligase ligand;
   or a pharmaceutically acceptable salt of either of these formulae.

In some embodiments, the compounds are further defined as:

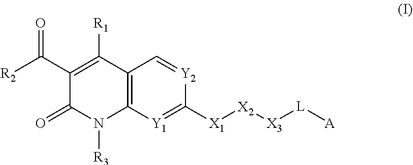

wherein:
R₁ is alkyl_{(C≤12)}, cycloalkyl_{(C≤12)}, aryl_{(C≤12)}, or a substituted version of any of these groups;
R₂ is alkyl_{(C≤12)}, cycloalkyl_{(C≤12)}, or a substituted version of either of these groups;
R₃ is cycloalkyl_{(C≤12)}, aryl_{(C≤12)}, or a substituted version of either of these groups;
Y₁ and Y₂ are each independently N or CH;
X₁ is O, S, or NRₐ,
   Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:

—(C(O))_{d}(CH₂)ₐX₄—(CH₂)_{b}—Y₃—X₅—(CH₂)_{c}— (IA)
wherein:
   d is 0 or 1;
   a, b, or c is 0, 1, 2, 3, 4, 5, or 6;
   X₄ is —C(O)—, —NR_{b}—, heteroarenediyl_{(C≤12)} or substituted heteroarenediyl_{(C≤12)};
      wherein R_{b} and R_{c} are each independently selected from hydrogen, alkyl_{(C≤6)}, or substituted alkyl_{(C≤6)};
   X₅ is —C(O)—, —NR_{b}—, —C(O)NR_{c}—, alkanediyl_{(C≤12)}, substituted alkanediyl_{(C≤12)}, arenediyl_{(C≤12)}, substituted arenediyl_{(C≤12)}, heteroarenediyl_{(C≤12)}, or substituted heteroarenediyl_{(C≤12)};
      wherein R_{b} and R_{c} are each independently selected from hydrogen, alkyl_{(C≤6)}, or substituted alkyl_{(C≤6)};
   Y₃ is a covalent bond, alkanediyl_{(C≤12)}, substituted alkanediyl_{(C≤12)}, —(CH₂CH₂O)ₑ(CH₂)_{f}—, —C(O)NRₐ-alkanediyl_{(C≤12)}, or substituted —C(O)NR_{d}-alkanediyl_{(C≤12)};
   wherein:
      e is 1, 2, 3, 4, or 5;
      f is 0, 1, 2, 3, 4, or 5; and
      R_{d} is hydrogen, alkyl_{(C≤6)}, or substituted alkyl_{(C≤6)}; or
a linking group of the formula:

-(AA₁)ₓ- (IB)
wherein:
   AA₁ is an amino acid residue; and
   x is 1, 2, 3, 4, 5, or 6; and
A is hydrogen or an E3 ligase ligand;
   or a pharmaceutically acceptable salt thereof.

In some embodiments, the compounds are further defined as:

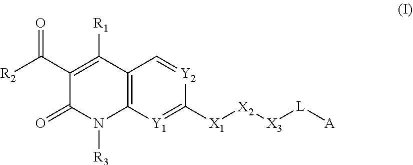

wherein:
R₁ is alkyl_{(C≤12)}, cycloalkyl_{(C≤12)}, aryl_{(C≤12)}, or a substituted version of any of these groups;
R₂ is alkyl_{(C≤12)}, cycloalkyl_{(C≤12)}, or a substituted version of either of these groups;
R₃ is cycloalkyl_{(C≤12)}, aryl_{(C≤12)}, or a substituted version of either of these groups;
Y₁ and Y₂ are each independently N or CH;
X₁ is O, S, or NRₐ,
   Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:

—C(O)(CH₂)ₐX₄—(CH₂)_{b}—Y₃—X₅—(CH₂)_{c}— (IC)
wherein:
   a, b, or c is 0, 1, 2, 3, 4, or 5;
   X₄ is —C(O)—, —NR_{b}—, heteroarenediyl_{(C≤12)} or substituted heteroarenediyl_{(C≤12)};
      wherein R_{b} and R_{c} are each independently selected from hydrogen, alkyl_{(C≤6)}, or substituted alkyl_{(C≤6)};
   X₅ is —C(O)—, —NR_{b}—, —C(O)NR_{c}—, heteroarenediyl_{(C≤12)} or substituted heteroarenediyl_{(C≤12)};
      wherein R_{b} and R_{c} are each independently selected from hydrogen, alkyl_{(C≤6)}, or substituted alkyl_{(C≤6)};
   Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
   wherein:
      d is 1, 2, 3, 4, or 5;
      e is 0, 1, 2, 3, 4, or 5; and
      R_{d} is hydrogen, alkyl_{(C≤6)}, or substituted alkyl_{(C≤6)}; or
a linking group of the formula:

-(AA₁)ₓ- (IB)
wherein:
   AA₁ is an amino acid residue; and
   x is 1, 2, 3, 4, 5, or 6; and
A is an E3 ligase ligand;
   or a pharmaceutically acceptable salt thereof.

In some embodiments, the compounds are further defined as:

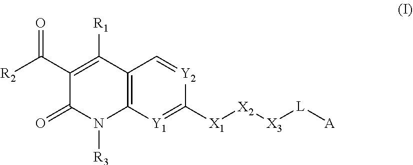

wherein:
R₁ is alkyl_{(C≤12)}, cycloalkyl_{(C≤12)}, aryl_{(C≤12)}, or a substituted version of any of these groups;
R₂ is alkyl_{(C≤12)}, cycloalkyl_{(C≤12)}, or a substituted version of either of these groups;
R₃ is cycloalkyl_{(C≤12)}, aryl_{(C≤12)}, or a substituted version of either of these groups;
Y₁ and Y₂ are each independently N or CH;
X₁ is O, S, or NRₐ,
   Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:

—C(O)(CH₂)ₐX₄—(CH₂)_{b}—Y₃—X₅—(CH₂)_{c}— (IC)
wherein:
   a, b, or c is 0, 1, 2, 3, 4, or 5;
   X₄ is —C(O)—, —NR_{b}—, heteroarenediyl_{(C≤12)} or substituted heteroarenediyl_{(C≤12)};
      wherein R_{b} and R_{c} are each independently selected from hydrogen, alkyl_{(C≤6)}, or substituted alkyl_{(C≤6)};
   X₅ is —C(O)—, —NR_{b}—, —C(O)NR_{c}—, heteroarenediyl_{(C≤12)} or substituted heteroarenediyl_{(C≤12)};
      wherein R_{b} and R_{c} are each independently selected from hydrogen, alkyl_{(C≤6)}, or substituted alkyl_{(C≤6)};
   Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
   wherein:
      d is 1, 2, 3, 4, or 5;
      e is 0, 1, 2, 3, 4, or 5; and
      R_{d} is hydrogen, alkyl_{(C≤6)}, or substituted alkyl_{(C≤6)}; and
A is an E3 ligase ligand;
   or a pharmaceutically acceptable salt thereof.

In some embodiments, the compounds are further defined as:

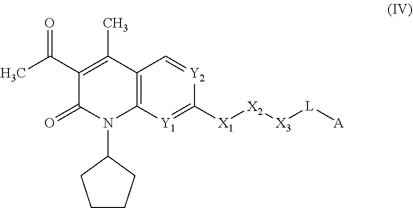

wherein:
Y₁ and Y₂ are each independently N or CH;
X₁ is O, S, or NRₐ,
   Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:

—C(O)(CH₂)ₐX₄—(CH₂)_{b}—Y₃—X₅—(CH₂)_{c}— (IC)
wherein:
   a, b, or c is 0, 1, 2, 3, 4, or 5;
   X₄ is —C(O)—, —NR_{b}—, heteroarenediyl_{(C≤12)} or substituted heteroarenediyl_{(C≤12)};
      wherein R_{b} and R_{c} are each independently selected from hydrogen, alkyl_{(C≤6)}, or substituted alkyl_{(C≤6)};
   X₅ is —C(O)—, —NR_{b}—, —C(O)NR_{c}—, heteroarenediyl_{(C≤12)} or substituted heteroarenediyl_{(C≤12)};
      wherein R_{b} and R_{c} are each independently selected from hydrogen, alkyl_{(C≤6)}, or substituted alkyl_{(C≤6)};
   Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
   wherein:
      d is 1, 2, 3, 4, or 5;
      e is 0, 1, 2, 3, 4, or 5; and
      R_{d} is hydrogen, alkyl_{(C≤6)}, or substituted alkyl_{(C≤6)}; and
A is an E3 ligase ligand;
   or a pharmaceutically acceptable salt thereof.

In some embodiments, the compounds are further defined as:

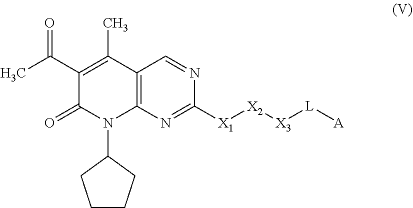

wherein:
X₁ is O, S, or NRₐ,
   Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:

—C(O)(CH₂)ₐX₄—(CH₂)_{b}—Y₃—X₅—(CH₂)_{c}— (IC)
wherein:
   a, b, or c is 0, 1, 2, 3, 4, or 5;
   X₄ is —C(O)—, —NR_{b}—, heteroarenediyl_{(C≤12)} or substituted heteroarenediyl_{(C≤12)};
      wherein R_{b} and R_{c} are each independently selected from hydrogen, alkyl_{(C≤6)}, or substituted alkyl_{(C≤6)};
   X₅ is —C(O)—, —NR_{b}—, or —C(O)NR_{c}—;
      wherein R_{b} and R_{c} are each independently selected from hydrogen, alkyl_{(C≤6)}, or substituted alkyl_{(C≤6)};
   Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
   wherein:
      d is 1, 2, 3, 4, or 5;
      e is 0, 1, 2, 3, 4, or 5; and
      R_{d} is hydrogen, alkyl_{(C≤6)}, or substituted alkyl_{(C≤6)}; and
A is an E3 ligase ligand;
   or a pharmaceutically acceptable salt thereof.

In some embodiments, the compounds are further defined as:

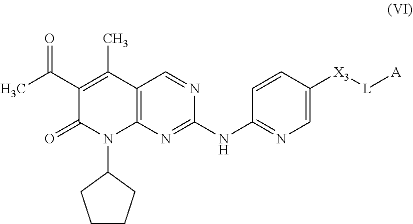

wherein:
X₃ is heterocycloalkanediyl_{(C≤12)} or substituted heterocycloalkanediyl_{(C≤12)};
L is a linking group of the formula:

—C(O)(CH₂)ₐX₄—(CH₂)_{b}—Y₃—X₅—(CH₂)_{c}— (IC)
wherein:
   a, b, or e is 0, 1, 2, 3, 4, or 5;
   X₄ is —C(O)—, —NR_{b}—, heteroarenediyl_{(C≤12)} or substituted heteroarenediyl_{(C≤12)};
      wherein R_{b} and R_{c} are each independently selected from hydrogen, alkyl_{(C≤6)}, or substituted alkyl_{(C≤6)};
   X₅ is —C(O)—, —NR_{b}—, or —C(O)NR_{c}—;
      wherein R_{b} and R_{c} are each independently selected from hydrogen, alkyl_{(C≤6)}, or substituted alkyl_{(C≤6)};
   Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
   wherein:
      d is 1, 2, 3, 4, or 5;
      e is 0, 1, 2, 3, 4, or 5; and
      R_{d} is hydrogen, alkyl_{(C≤6)}, or substituted alkyl_{(C≤6)}; and
A is an E3 ligase ligand;
   or a pharmaceutically acceptable salt thereof.

In some embodiments, the compounds are further defined as:

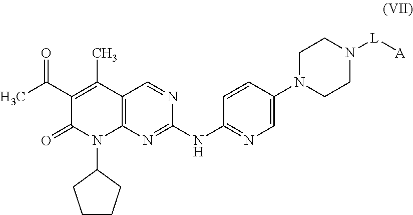

wherein:
L is a linking group of the formula:

—C(O)—(CH₂)ₐX₄—(CH₂)_{b}—Y₃—X₅—(CH₂)_{c}— (IC)
wherein:
   a, b, or e is 0, 1, 2, 3, 4, or 5; provided the sum of a, b, and c are greater than 1;
   X₄ is —C(O)—, —NR_{b}—, heteroarenediyl_{(C≤12)} or substituted heteroarenediyl_{(C≤12)};
      wherein R_{b} and R_{c} are each independently selected from hydrogen, alkyl_{(C≤6)}, or substituted alkyl_{(C≤6)};
   X₅ is —C(O)—, —NR_{b}—, or —C(O)NR_{c}—;
      wherein R_{b} and R_{c} are each independently selected from hydrogen, alkyl_{(C≤6)}, or substituted alkyl_{(C≤6)};
   Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
   wherein:
      d is 1, 2, 3, 4, or 5;
      e is 0, 1, 2, 3, 4, or 5; and
      R_{d} is hydrogen, alkyl_{(C≤6)}, or substituted alkyl_{(C≤6)};
A is an E3 ligase ligand;
   or a pharmaceutically acceptable salt thereof.

In some embodiments, the compounds are further defined as:

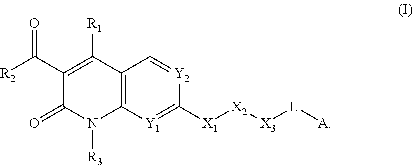

wherein:
R₁ is alkyl_{(C≤12)}, cycloalkyl_{(C≤12)}, aryl_{(C≤12)}, or a substituted version of any of these groups;
R₂ is alkyl_{(C≤12)}, cycloalkyl_{(C≤12)}, or a substituted version of either of these groups;
R₃ is cycloalkyl_{(C≤12)}, aryl_{(C≤12)}, or a substituted version of either of these groups;
Y₁ and Y₂ are each independently N or CH;
X₁ is O, S, or NRₐ,
   Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:

-(AA₁)ₓ- (IB)
wherein:
   AA₁ is an amino acid residue; and
   x is 1, 2, 3, 4, 5, or 6;
A is an E3 ligase ligand;
   or a pharmaceutically acceptable salt thereof.

In some embodiments, the compounds are further defined as:

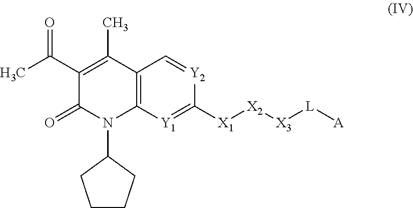

wherein:
Y₁ and Y₂ are each independently N or CH;
X₁ is O, S, or NRₐ,
   Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:

-(AA₁)ₓ- (IB)
wherein:
   AA₁ is an amino acid residue; and
   x is 1, 2, 3, 4, 5, or 6;
A is an E3 ligase ligand;
   or a pharmaceutically acceptable salt thereof.

In some embodiments, the compounds are further defined as:

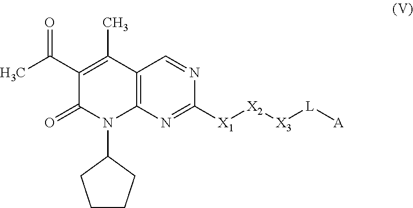

wherein:
X₁ is O, S, or NRₐ,
   Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:

-(AA₁)ₓ- (IB)
wherein:
   AA₁ is an amino acid residue; and
   x is 1, 2, 3, 4, 5, or 6;
A is an E3 ligase ligand;
   or a pharmaceutically acceptable salt thereof.

In some embodiments, the compounds are further defined as:

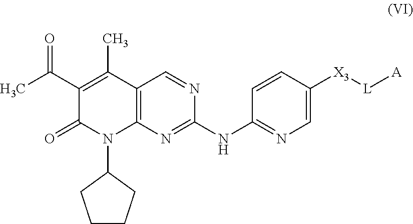

wherein:
X₃ is heterocycloalkanediyl_{(C≤12)} or substituted heterocycloalkanediyl_{(C≤12)};
L is a linking group of the formula:

-(AA₁)ₓ- (IB)
wherein:
   AA₁ is an amino acid residue; and
   x is 1, 2, 3, 4, 5, or 6;
A is an E3 ligase ligand;
   or a pharmaceutically acceptable salt thereof.

In some embodiments, the compounds are further defined as:

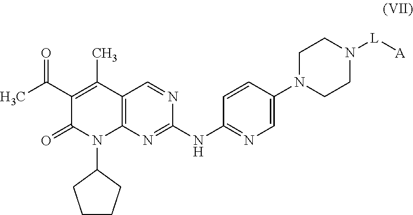

wherein:
L is a linking group of the formula:

-(AA₁)ₓ- (IB)
wherein:
   AA₁ is an amino acid residue; and
   x is 1, 2, 3, 4, 5, or 6;
A is an E3 ligase ligand;
   or a pharmaceutically acceptable salt thereof.

In other embodiments, the compounds are further defined as:

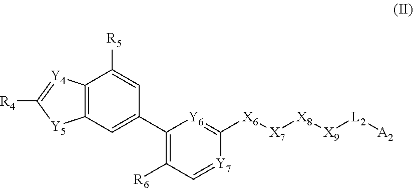

wherein:
R₄ is hydrogen, alkyl_{(C≤12)}, substituted alkyl_{(C≤12)}, cycloalkyl_{(C≤12)}, or substituted cycloalkyl_{(C≤12)};
R₅ and R₆ are each independently is hydrogen, halo, alkyl_{(C≤12)}, substituted alkyl_{(C≤12)}, cycloalkyl_{(C≤12)}, or substituted cycloalkyl_{(C≤12)};
Y₄, Y₆, and Y₇ are each independently N or CH;
Y₅ is O, S, or NR_{d}, wherein:
   R_{d} is hydrogen, alkyl_{(C≤12)}, substituted alkyl_{(C≤12)}, cycloalkyl_{(C≤12)}, or substituted cycloalkyl_{(C≤12)};
X₆ is O, S, or NRₑ,
   Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:

—(CH₂)_{g}X₁₀—(CH₂)ₕ—Y₈—X₁₁—(CH₂)ᵢ— (IIA)
wherein:
   g, h, and i are each independently 0, 1, 2, 3, 4, or 5;
   X₁₀ is —C(O)—, —NR_{f}—, heteroarenediyl_{(C≤12)} or substituted heteroarenediyl_{(C≤12)}, wherein:
      R_{f} is hydrogen, alkyl_{(C≤6)}, or substituted alkyl_{(C≤6)};
   X₁₁ is —C(O)—, —NR_{f}—, —C(O)NR_{g}—, heteroarenediyl_{(C≤12)} or substituted heteroarenediyl_{(C≤12)};
      wherein R_{f} and R_{g} are each independently selected from hydrogen, alkyl_{(C≤6)}, or substituted alkyl_{(C≤6)};
   Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
   wherein:
      j is 1, 2, 3, 4, or 5;
      k is 0, 1, 2, 3, 4, or 5; and
      R_{g} is hydrogen, alkyl_{(C≤6)}, or substituted alkyl_{(C≤6)}; or
a linking group of the formula:

-(AA₂)_{y}- (IIB)
wherein:
   AA₂ is an amino acid residue; and
   y is 1, 2, 3, 4, 5, or 6; and
A₂ is hydrogen or an E3 ligase ligand;
   or a pharmaceutically acceptable salt thereof.

In some embodiments, the compounds are further defined as:

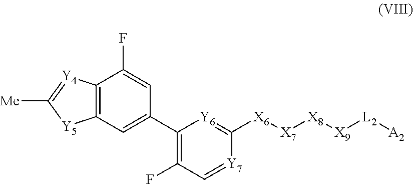

wherein:
Y₄, Y₆, and Y₇ are each independently N or CH;
Y₅ is O, S, or NR_{d}, wherein:
   R_{d} is hydrogen, alkyl_{(C≤12)}, substituted alkyl_{(C≤12)}, cycloalkyl_{(C≤12)}, or substituted cycloalkyl_{(C≤12)};
X₆ is O, S, or NRₑ,
   Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:

—(CH₂)_{g}X₁₀—(CH₂)ₕ—Y₈—X₁₁—(CH₂)ᵢ— (IIA)
wherein:
   g, h, and i are each independently 0, 1, 2, 3, 4, or 5;
   X₁₀ is —C(O)—, —NR_{f}—, heteroarenediyl_{(C≤12)} or substituted heteroarenediyl_{(C≤12)}, wherein:
      R_{f} is hydrogen, alkyl_{(C≤6)}, or substituted alkyl_{(C≤6)};
   X₁₁ is —C(O)—, —NR_{f}—, —C(O)NR_{g}, heteroarenediyl_{(C≤12)} or substituted heteroarenediyl_{(C≤12)};
      wherein R_{f} and R_{g} are each independently selected from hydrogen, alkyl_{(C≤6)}, or substituted alkyl_{(C≤6)};
   Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
   wherein:
      j is 1, 2, 3, 4, or 5;
      k is 0, 1, 2, 3, 4, or 5; and
      R_{g} is hydrogen, alkyl_{(C≤6)}, or substituted alkyl_{(C≤6)}; or
a linking group of the formula:

-(AA₂)_{y}- (IIB)
wherein:
   AA₂ is an amino acid residue; and
   y is 1, 2, 3, 4, 5, or 6; and
A₂ is hydrogen or an E3 ligase ligand;
   or a pharmaceutically acceptable salt thereof.

In some embodiments, the compounds are further defined as:

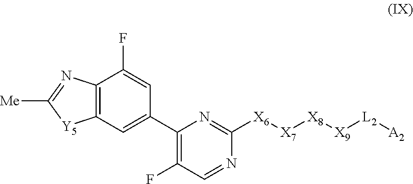

wherein:
Y₅ is O, S, or NR_{d}, wherein:
   R_{d} is hydrogen, alkyl_{(C≤12)}, substituted alkyl_{(C≤12)}, cycloalkyl_{(C≤12)}, or substituted cycloalkyl_{(C≤12)};
X₆ is O, S, or NRₑ,
   Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:

—(CH₂)_{g}X₁₀—(CH₂)ₕ—Y₈—X₁₁—(CH₂)ᵢ— (IIA)
wherein:
   g, h, and i are each independently 0, 1, 2, 3, 4, or 5;
   X₁₀ is —C(O)—, —NR_{f}—, heteroarenediyl_{(C≤12)} or substituted heteroarenediyl_{(C≤12)}, wherein:
      R_{f} is hydrogen, alkyl_{(C≤6)}, or substituted alkyl_{(C≤6)};
   X₁₁ is —C(O)—, —NR_{f}—, —C(O)NR_{g}, heteroarenediyl_{(C≤12)} or substituted heteroarenediyl_{(C≤12)};
      wherein R_{f} and R_{g} are each independently selected from hydrogen, alkyl_{(C≤6)}, or substituted alkyl_{(C≤6)};
   Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
   wherein:
      j is 1, 2, 3, 4, or 5;
      k is 0, 1, 2, 3, 4, or 5; and
      R_{g} is hydrogen, alkyl_{(C≤6)}, or substituted alkyl_{(C≤6)}; or
a linking group of the formula:

-(AA₂)_{y}- (IIB)
wherein:
   AA₂ is an amino acid residue; and
   y is 1, 2, 3, 4, 5, or 6; and
A₂ is hydrogen or an E3 ligase ligand;
   or a pharmaceutically acceptable salt thereof.

In some embodiments, the compounds are further defined as:

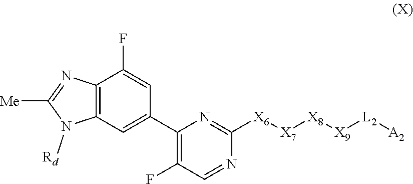

wherein:
R_{d} is hydrogen, alkyl_{(C≤12)}, substituted alkyl_{(C≤12)}, cycloalkyl_{(C≤12)}, or substituted cycloalkyl_{(C≤12)};
X₆ is O, S, or NRₑ,
   Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:

—(CH₂)_{g}X₁₀—(CH₂)ₕ—Y₈—X₁₁—(CH₂)ᵢ— (IIA)
wherein:
   g, h, and i are each independently 0, 1, 2, 3, 4, or 5;
   X₁₀ is —C(O)—, —NR_{f}—, heteroarenediyl_{(C≤12)} or substituted heteroarenediyl_{(C≤12)}, wherein:
      R_{f} is hydrogen, alkyl_{(C≤6)}, or substituted alkyl_{(C≤6)};
   X₁₁ is —C(O)—, —NR_{f}—, —C(O)NR_{g}—, heteroarenediyl_{(C≤12)} or substituted heteroarenediyl_{(C≤12)};
      wherein R_{f} and R_{g} are each independently selected from hydrogen, alkyl_{(C≤6)}, or substituted alkyl_{(C≤6)};
   Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
   wherein:
      j is 1, 2, 3, 4, or 5;
      k is 0, 1, 2, 3, 4, or 5; and
      R_{g} is hydrogen, alkyl_{(C≤6)}, or substituted alkyl_{(C≤6)}; or
a linking group of the formula:

-(AA₂)_{y}- (IIB)
wherein:
   AA₂ is an amino acid residue; and
   y is 1, 2, 3, 4, 5, or 6; and
A₂ is hydrogen or an E3 ligase ligand;
   or a pharmaceutically acceptable salt thereof.

In some embodiments, the compounds are further defined as:

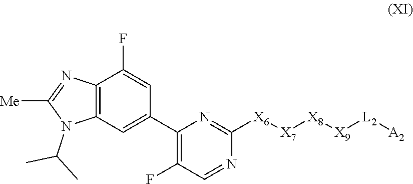

wherein:
X₆ is O, S, or NRₑ,
   Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:

—(CH₂)_{g}X₁₀—(CH₂)ₕ—Y₈—X₁₁—(CH₂)ᵢ— (IIA)
wherein:
   g, h, and i are each independently 0, 1, 2, 3, 4, or 5;
   X₁₀ is —C(O)—, —NR_{f}—, heteroarenediyl_{(C≤12)} or substituted heteroarenediyl_{(C≤12)}, wherein:
      R_{f} is hydrogen, alkyl_{(C≤6)}, or substituted alkyl_{(C≤6)};
   X₁₁ is —C(O)—, —NR_{f}—, —C(O)NR_{g}—, heteroarenediyl_{(C≤12)} or substituted heteroarenediyl_{(C≤12)};
      wherein R_{f} and R_{g} are each independently selected from hydrogen, alkyl_{(C≤6)}, or substituted alkyl_{(C≤6)};
   Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
   wherein:
      j is 1, 2, 3, 4, or 5;
      k is 0, 1, 2, 3, 4, or 5; and
      R_{g} is hydrogen, alkyl_{(C≤6)}, or substituted alkyl_{(C≤6)}; or
a linking group of the formula:

-(AA₂)_{y}- (IIB)
wherein:
   AA₂ is an amino acid residue; and
   y is 1, 2, 3, 4, 5, or 6; and
A₂ is hydrogen or an E3 ligase ligand;
   or a pharmaceutically acceptable salt thereof.

In some embodiments, a is 0, 1, 2, or 3. In some embodiments a is 0 or 1. In other embodiments, a is 1 or 2. In other embodiments, a is 6. In some embodiments, b is 0, 1, 2, or 3. In some embodiments, b is 0 or 1. In other embodiments, b is 1 or 2. In some embodiments, c is 0, 1, 2, or 3. In some embodiments, c is 0 or 1. In other embodiments, c is 1 or 2. In some embodiments, d is 0. In other embodiments, d is 1.

In some embodiments, X₄ is heteroarenediyl_{(C≤12)} or substituted heteroarenediyl_{(C≤12)} such as 1,2,3-triazol-1,4-diyl. In other embodiments, X₄ is NR_{b} such as NH or N(CH₃). In some embodiments, X₅ is —C(O)NR_{c}—; wherein R_{c} is hydrogen, alkyl_{(C≤6)}, or substituted alkyl_{(C≤6)} such as —C(O)NH— or —C(O)—.

In some embodiments, Y₃ is a covalent bond. In other embodiments, Y₃ is alkanediyl_{(C≤8)} or substituted alkanediyl_{(C≤8)} such as methanediyl, ethanediyl, propanediyl, or butanediyl. In other embodiments, Y₃ is —C(O)NRₐ-alkanediyl_{(C≤12)} or substituted —C(O)NR_{d}-alkanediyl_{(C≤12)} such as —C(O)NH-alkanediyl_{(C≤12)} or substituted —C(O)NH-alkanediyl_{(C≤12)}. In some embodiments, the alkanediyl_{(C≤12)} or substituted alkanediyl_{(C≤12)} is methanediyl, ethanediyl, propanediyl, butanediyl, pentanediyl, or hexanediyl. In other embodiments, Y₃ is —(CH₂CH₂O)_{d}(CH₂)ₑ—, wherein: e is 1, 2, 3, 4, or 5; and f is 0, 1, 2, 3, 4, or 5. In some embodiments, e is 2, 3, or 4. In some embodiments, f is 0 or 1.

In some embodiments, AA₁ is a canonical amino acid. In some embodiments, x is 1, 2, or 3. In some embodiments, X₆ is NRₑ, wherein Rₑ is hydrogen, alkyl_{(C≤6)}, or substituted alkyl_{(C≤6)}. In some embodiments, X₇ is pyridinediyl such as 2,5-pyridinediyl. In some embodiments, X₈ is alkanediyl_{(C≤6)} such as methylene. In some embodiments, X₉ is heterocycloalkanediyl_{(C≤6)} such as 1,4-piperazindiyl. In some embodiments, g is 0, 1, or 2. In some embodiments, g is 2. In some embodiments, h is 0, 1, or 2. In some embodiments, h is 0. In some embodiments, i is 0, 1, or 2. In some embodiments, i is 1.

In some embodiments, X₁₀ is —NR_{f}—. In some embodiments, R_{f} is hydrogen. In some embodiments, Y_{g} is a covalent bond. In some embodiments, X₁₁ is —C(O)—.

In some embodiments, A is hydrogen. In other embodiments, A is an E3 ligase ligand for VHL, MDM2, cereblon, or cIAP. In further embodiments, the E3 ligase ligand is pomalidomide, thalidomide, lenalidomide, VHL-1, adamantane, 1-((4,4,5,5,5-pentafluoropentyl)sulfinyl)nonane, nutlin-3a, RG7112, RG7338, AMG 232, AA-115, bestatin, MV-1, LCL161, or a derivative thereof.

In some embodiments, A₂ is hydrogen. In other embodiments, A₂ is an E3 ligase ligand for VHL, MDM2, cereblon, or cIAP. In further embodiments, the E3 ligase ligand is pomalidomide, thalidomide, lenalidomide, VHL-1, adamantane, 1-((4,4,5,5,5-pentafluoropentyl)sulfinyl)nonane, nutlin-3a, RG7112, RG7338, AMG 232, AA-115, bestatin, MV-1, LCL161, or a derivative thereof. In still further embodiments, the E3 ligase ligand is:

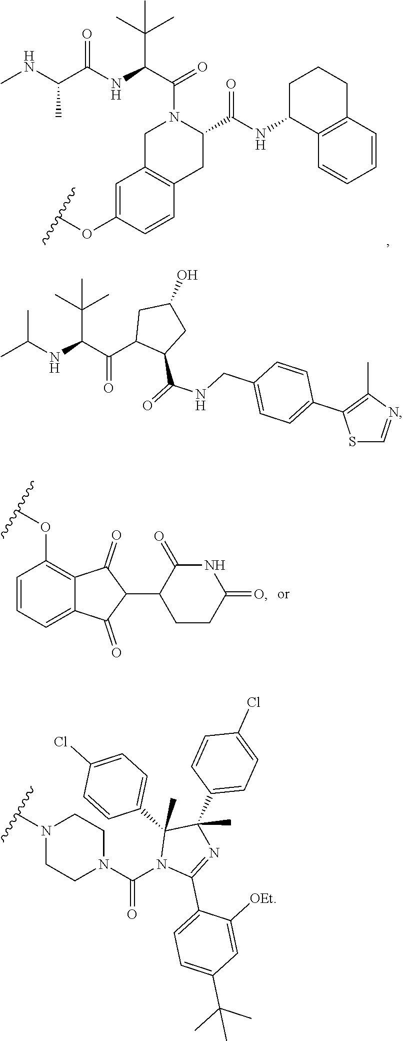

In some embodiments, the compound is further defined as:

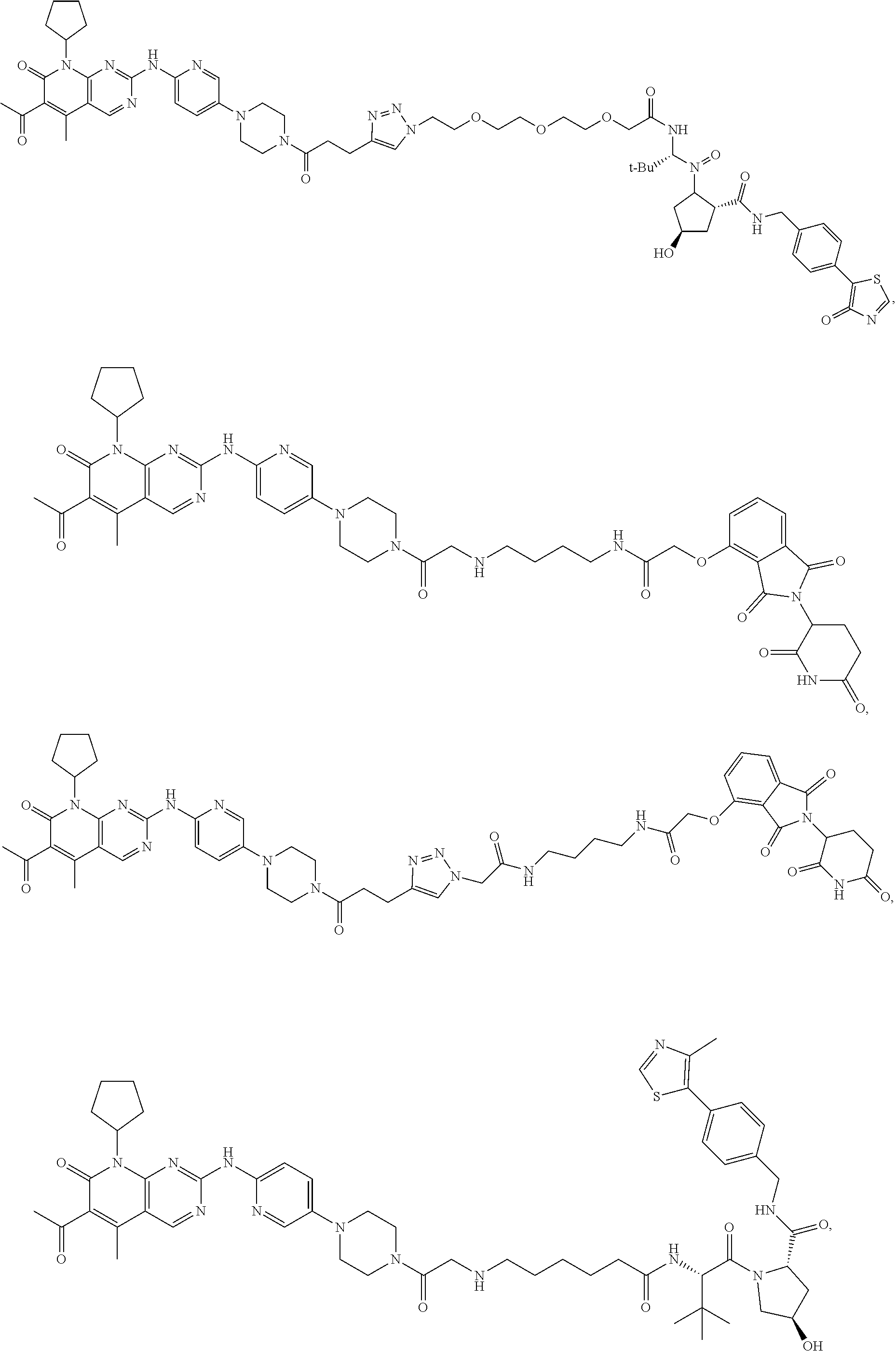

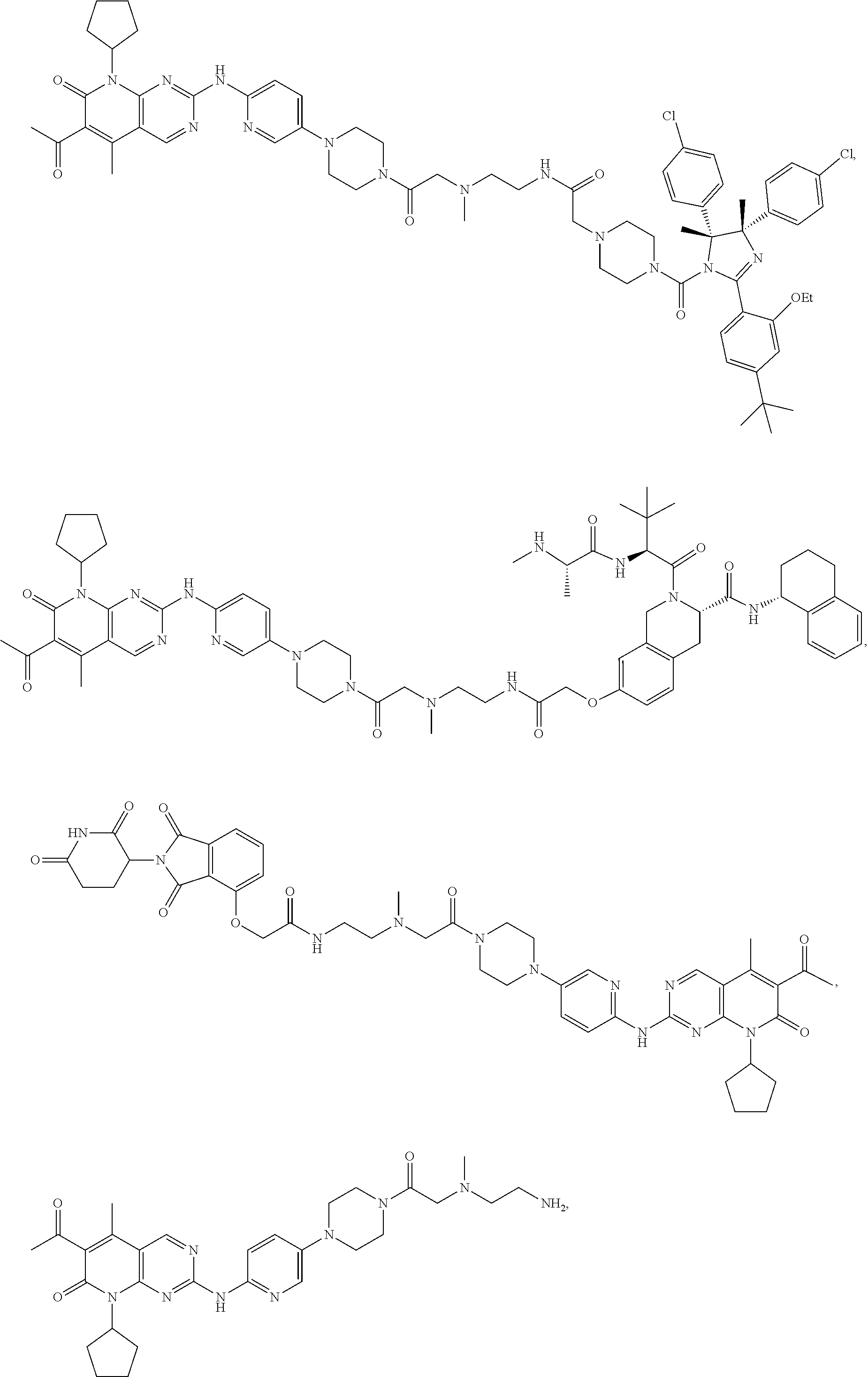

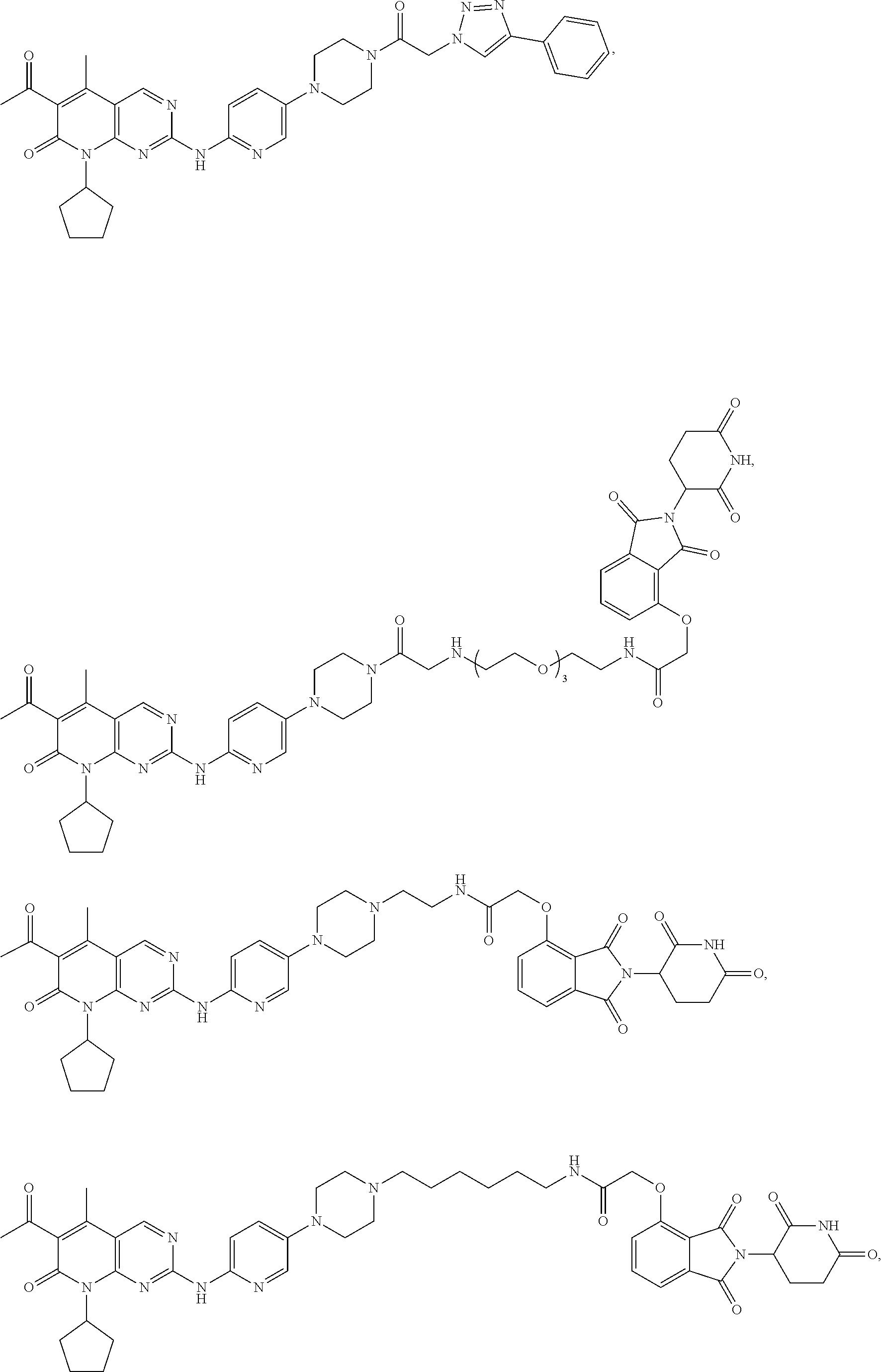

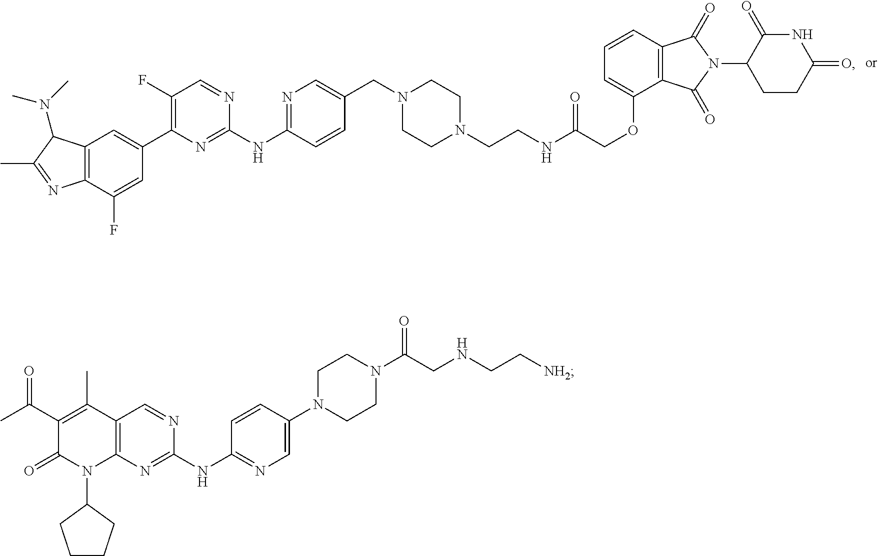 [SMILES: CC1=Nc2c(F)cc(-c3nc(Nc4ccc(CN5CCN(CCNC(=O)COc6cccc7c6C(=O)N(C6CCC(=O)NC6=O)C7=O)CC5)cn4)ncc3F)cc2C1N(C)C ; CC(=O)c1c(C)c2cnc(Nc3ccc(N4CCN(C(=O)CNCCN)CC4)cn3)nc2n(C2CCCC2)c1=O]

or a pharmaceutically acceptable salt thereof.

In some embodiments, the compound is further defined as.

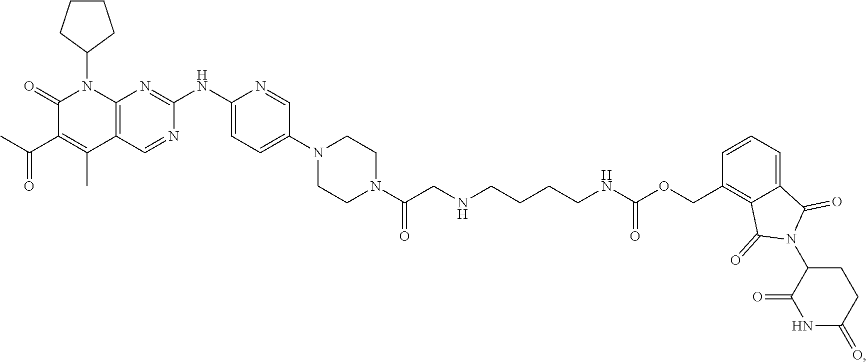 [SMILES: CC(=O)c1c(C)c2cnc(Nc3ccc(N4CCN(C(=O)CNCCCCNC(=O)OCc5cccc6c5C(=O)N(C5CCC(=O)NC5=O)C6=O)CC4)cn3)nc2n(C2CCCC2)c1=O]

or a pharmaceutically acceptable salt thereof.

In another aspect, the present disclosure provides compositions comprising a compound of the present disclosure and an excipient. In some embodiments, the composition is formulated for administration orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In some embodiments, the composition is formulated as a unit dose.

In yet another aspect, the present disclosure provides methods of treating a disease or disorder in a patient comprising administering a therapeutically effective amount of a compound or composition of the present disclosure to the patient. In some embodiments, the disease or disorder is cancer. In some embodiments, the cancer has aberrant signaling of CDK4 or CDK6. In some embodiments, the cancer is a leukemia, breast cancer, gastric cancer, pancreatic cancer, or liver cancer. In further embodiments, the leukemia is acute lymphoblastic leukemia, acute myeloid leukemia, or chronic myeloid leukemia. In some embodiments, the method further comprises administering a second anti-cancer therapy. In some embodiments, the patient is a mammal, such as a human.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.1%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows inhibition of CDK4 and CDK6 kinase activity for YX-2-79, YX-2-107, and YX-2-115.

FIGS. 2A-2D show the effect of palbociclib, YX-2-107 and Cereblon-ligand (CRBN-L) in BV173 and SUP-B15 cells. FIGS. 2A & 2B show cell cycle of BV173 cells (FIG. 2A) and SUP-B15 cells (FIG. 2B) after a 48 h treatment with the indicated doses of drugs. FIGS. 2C & 2D show western blot of BV173 cells (FIG. 2C) and SUP-B15 cells (FIG. 2D) showing the expression of CDK6, CDK4, FOXM1 and phosphorylation of RB after a 72 h treatment with the indicated doses of drugs.

FIGS. 3A & 3B show YX-2-107 induces rapid proteasome-dependent degradation of CDK6. FIG. 3A shows immunoblot for CDK6 expression in BV173 cells treated for the indicated times with YX-2-107 or palbociclib. FIG. 3B shows immunoblot for CDK6 expression in BV173 cells treated with YX-2-107 with or without the proteasomal inhibitor MG132 for 4 hours.

FIGS. 4A-4C show in vivo treatment with YX-2-107 or palbociclib. Leukemic mice, 3 per group, were treated with palbociclib or YX-2-107 at 150 mg/kg for 3 consecutive days. 24 hours after the end of the treatment, bone marrow cells were purified (purity of human cells was >90% by CD19-CD10 flow cytometry) and subjected to cell cycle analysis by propidium iodide staining (FIG. 4A) or western blot for RB-phosphorylation and FOXM1, CDK4, and CDK6 expression (FIG. 4B). FIG. 4C shows densitometry of CDK4 and CDK6 expression from FIG. 4B.

FIGS. 5A-5C show effects of YX-2-233 in Ph+ ALL cell lines. FIG. 5A shows the structure of YX-2-233. FIG. 5B shows cell cycle analysis at 24 h. FIG. 5C shows immunoblot of YX-2-233-treated (24 h) BV173 or SUP-B15 cells.

FIG. 6 shows the comparison of effects between YX-2-196 and YX-2-107 in BV173 and SUP-B15 cells.

FIG. 7 shows the comparison of effects between AC-1-027 and YX-2-107 in BV173 and SUP-B15 cells after 4 h and 24 h.

FIG. 8 shows results from in vivo experiment to compare the effects on leukemia load post 10 days treatment with daily IP injections of palbociclib and YX-2-107.

FIG. 9 shows additional data for YX-2-107 in Ph+BV173 and SUP-B15 cells.

FIG. 10A-10E show the effect of CDK6 silencing on apoptosis and leukemogenesis of BV173 cells. BV173 cells were transduced with scramble (SCR), CDK4 or CDK6 (82, 86, 88, 73) shRNA vectors and selected with puromycin or treated with Palbociclib (2 μM). (FIG. 10A) Cell cycle analysis by propidium iodide staining of shRNA-transduced or Palbociclib-treated cells; (FIG. 10B) Apoptosis detected by Annexin V staining after 7 days of puromycin or Palbociclib treatment; (FIG. 10C) Representative immunoblot for CDK4/6 and phospho-RB expression; (FIG. 10D) Apoptosis detected by Annexin V staining of BV173 cells transduced with TET-ON shCDK6-88 and treated with doxycycline (1 μg/ml) or Palbociclib (1 μM) for 7 days; (FIG. 10E) leukemia load (peripheral blood flow cytometry analysis of CD19+mCherry+ cells performed two weeks after treatment cessation) of NSG mice injected with BV173 TET-ON shCDK6-88 cells and left untreated or treated with DOX (2 g/L in the drinking water) or Palbociclib chow for 4 weeks starting 7 days post-cell injection; (FIG. 10F) Kaplan-Meier survival plot of NSG mice injected with BV173 TET-ON shCDK6-88 and left untreated or treated with doxycycline (2 g/L in the drinking water) or Palbociclib chow for four weeks starting 7 days post-cell injection.

FIGS. 11A-11D show Specific effects of CDK6 silencing on the cell cycle and apoptosis of BV173 cells. (FIG. 11A) Cell cycle analysis of BV173 cells transduced with TET-ON shCDK6-88 or the empty vector and treated with DMSO, DOX (1 μg/ml) or Palbociclib (1 μM) for 2 days or BV173 shCDK6-88 expressing a shRNA resistant form of CDK6 (CDK6-shRES) treated with DOX (1 μM) for 2 days; (FIG. 11B) CDK6 and phospho-RB levels in BV173 shCDK6-88 EV or CDK6-shRES cells treated with DMSO, DOX (1 μM) or Palbociclib (1 μM) for 3 days; (FIG. 11C) Apoptosis monitored by Annexin V staining of BV173 cells transduced with TET-ON shCDK6-88, empty (EV) vector, or a p53-targeting shRNA 9sh-p53) and treated with DOX (1 μg/ml) or Palbociclib (1 μM) for 10 days; (FIG. 11D) p53 levels in EV or sh-p53-transduced BV173 cells.

FIGS. 12A & 12B show apoptosis induced by CDK6 silencing in BV173 cells is largely p53-independent. (FIG. 12A) Apoptosis as monitored by Annexin V staining of BV173 cells transduced with TET-ON shCDK6-88, empty (EV) vector, or a p53-targeting shRNA 9sh-p53) and treated with DOX (1 μg/ml) or Palbociclib (1 μM) for 10 days; (FIG. 12B) immunoblot for p53 of EV or sh-p53-transduced BV173 cells.

FIG. 13 shows the effect of CDK6 silencing versus enzymatic inhibition for engraftment of BV173 cells in NSG mice. Peripheral blood flow cytometry analysis performed two weeks after treatment cessation of NSG mice injected with BV173 TET-ON shCDK6-88 cells and left untreated or treated with DOX (2 g/L in the drinking water) or Palbociclib chow for 4 weeks starting 7 days post-cell injection.

FIGS. 14A-14D show gene subset regulated by CDK6 silencing not by kinase inhibition in BV173 cells. (FIG. 14A) Heat-map showing genes selectively regulated by CDK6 silencing as compared to Palbociclib treatment in Ph+BV173 cells; (FIG. 14B) Heat-map of 8 genes selectively downregulated by CDK6 silencing; (FIG. 14C) qPCR analysis of selected genes differentially regulated by CDK6 silencing but not Palbociclib treatment in Ph+BV173 cells. Data represent mean+SD of three independent experiments. Statistical analysis: one way ANOVA with Bonferroni's correction. *<0.05 **<0.01, ***<0.001; (FIG. 14D) plots of the correlation between the expression of CDK6 and HDAC1 or CDK6 and SMARCD2 in a panel of 122 Ph+ ALL samples (GSE13159; MILE, microarray innovations in leukemia).

FIGS. 15A-15D show Palbociclib and derivatives. (FIG. 15A) Palbociclib and derivative compounds with differences in kinase inhibition due to modest changes to the piperazine-linker tail; (FIG. 15B) Several PROTAC candidates using various linkers and either a VHL or a Cereblon recruiting ligand; (FIG. 15C) YX-2-107, a CRBN-Palbociclib PROTAC, selectively degrades CDK6 in BV173 cells after a 4-h treatment; (FIG. 15D) Synthesis of CRBN E3-Amine component for Cereblon E3 ligase recruitment and as a control.

FIG. 16 shows schematic steps in the synthesis of PROTAC YX-2-107.

FIGS. 17A-17E show proteasome-dependent degradation and CDK6 stability in PROTAC YX-2-107-treated cells. Immunoblot shows CDK6 expression in BV173 cells treated with: (FIG. 17A) PROTAC YX-2-107 or Palbociclib; (FIG. 17B) PROTAC YX-2-107 with or without the proteasomal inhibitor MG132 for 4 hours; (FIG. 17C) PROTAC YX-2-107 (2 μM) with Palbociclib or Thalidomide at the indicated concentrations for 4 hours; and (FIG. 17D) PROTAC YX-2-107 for 4 hours, washed and cultured without YX-2-107 for 1, 2, 4, 6, and 24 hours; (FIG. 17E) Volcano plot illustrates significantly differentially abundant proteins identified by at least two unique peptides found in all three replicates of the PROTAC-treated or control (DMSO-treated) samples. The −log 10 p-value is plotted against the log 2-fold change (PROTAC/DMSO). Blue points represent proteins with p<0.05 and an absolute fold-change >2.

FIGS. 18A-18C show the effects of PROTAC YX-2-233 in Ph+ ALL cell lines. (FIG. 18A) Structure of PROTAC YX-2-233; (FIG. 18B) cell cycle analysis at 24 h; and (FIG. 18C) immunoblot of PROTAC YX-2-233-treated (24 h) BV173 or SUP-B15 cells.

FIGS. 19A-19D show the effects of Palbociclib, YX-2-107 and Cereblon-ligand (CRBN-L) in Ph+BV173 and SUP-B15 cells. (FIGS. 19A & 19B) Cell cycle analysis of BV173 cells (FIG. 19A) and SUP-B15 cells (FIG. 19B) after a 48-h treatment with the indicated drug concentrations; (FIGS. 19C & 19D) Immunoblot of BV173 cells (FIG. 19C) and SUP-B15 cells (FIG. 19D) showing the expression of CDK6, CDK4, FOXM1, and phospho-RB after a 72 h treatment with the indicated drug concentrations.

FIGS. 20A-20I show the effects of PROTAC YX-2-107 in Ph+ ALL cells and normal hematopoietic progenitors. (FIGS. 20A & 20B) Cell cycle analysis of BV173 cells (FIG. 20A) and SUP-B15 cells (FIG. 20B) after a 48-h treatment with the indicated drug concentrations; (FIGS. 20C & 20D) Immunoblot of BV173 cells (FIG. 20C) and SUP-B15 cells (FIG. 20D) showing the expression of CDK6, CDK4, FOXM1, and phospho-RB after a 72 h treatment with the indicated drug concentrations; (FIG. 20E) immunoblot for CDK4/CDK6 expression (left) and number of S phase cells (represented as the percentage of drug-treated vs untreated cells taken as 100) (right) in YX-2-107-treated Ph+ ALL cells (sample #004); (FIGS. 20F-20H) immunoblot for CDK4/CDK6 expression and percentage of S phase cells in YX-2-107-treated normal hematopoietic progenitors and BV173 cells; (FIG. 20I) cell cycle profile of CD34+ HSPC transduced with anti-CDK4 or anti-CDK6 shRNA and selected with puromycin for 48 h or treated with Palbociclib (500 nM; 24 h).

FIGS. 21A & 21B show effects of CDK6-degrading PROTACs or Palbociclib on the proliferation of BV173 cells. (FIG. 21A) structure of YX-2-107-related PROTACs; (FIG. 21B) immunoblots of BV173 cells treated with PROTACs at the indicated concentrations for 24 h; (FIG. 21C) percentage of S phase cells by propidium iodide staining of BV173 cells treated with PROTACs as in (FIG. 21B) or with Palbociclib for 24 hour. IC₅₀s were calculated based on the percent reduction of S-phase cells using graphpad PRISM software.

FIG. 22A-22E show PROTAC YX-2-107 metabolic stability and its biological activity in a mouse xenograft of Ph+ ALL. (FIG. 22A) Half-life of YX-2-107, Palbociclib, and E3 ligase recruiting molecules incubated in mouse liver microsomes; (FIG. 22B) Time course of plasma concentration of YX-2-107 injected intraperitoneally at 10 mg/kg into C57BL/6j mice and its pharmacokinetic property (left); c-e) Cell cycle analysis by propidium iodide staining (FIG. 22C), and immunoblot for phospho-RB, FOXM1, CDK4 and CDK6 (FIG. 22D), with densitometry of CDK4 and CDK6 expression (FIG. 22E) of bone marrow leukemic cells (>90% CD19+CD10+ by flow cytometry) from NSG mice injected with Ph+ ALL cells and treated (3 mice/group) with Palbociclib or YX-2-107 at 150 mg/kg/day for 3 consecutive days when peripheral blood leukemic cells were 50%. Bone marrow leukemic cells were purified 24 hours after the cessation of drug treatment.

FIGS. 23A & 23B show in vivo effects of PROTAC AC-1-212 or Palbociclib on the proliferation of Ph+ ALL cells. Mice were injected with human Ph+ ALL cells (sample #004) and, when peripheral blood leukemic cells (CD19+CD10+) were >50%, treated with vehicle, PROTAC AC-1-212 20 mg/kg IP BID or Palbociclib 150 mg/Kg by gavage for 3 consecutive days. Twelve hours after the last treatment, bone marrow cells (>90% CD19+CD10+) were purified and assessed for the percentage of S phase cells (FIG. 23A) or expression of CDK4/6, phospho-RB and FOXM1 (FIG. 23B). Quantitation of CDK4 and CDK6 levels (based on FIG. 23B immunoblot) is shown in FIG. 23C.

FIGS. 24A-24D show the effect of PROTAC YX-2-107 treatment on normal mouse hematopoiesis. 6 (2 month-old) C57BL/6j mice were treated with vehicle (Veh) or PROTAC YX-2-107 (107) 150 mg/kg IP daily for 10 consecutive days. 4 days after the cessation of treatment, peripheral blood (PB) and bone marrow (BM) cells were collected and analyzed by flow cytometry; (FIG. 24A) gating strategy for the quantification of stem and progenitor cells; (FIG. 24B) gating strategy for the quantification of B-lymphoid progenitor cells; (FIG. 24C) percentage of progenitor populations in the BM, (FIG. 24D) number of selected hematopoietic cells in the PB. p-value was considered non-significant (N.S.) if >0.05.

FIGS. 25A-25J show leukemia load in mice injected with Ph+ ALL primary samples and treated with PROTAC YX-2-107. NSG mice injected with primary Ph+ ALL-004 (FIGS. 25A-25E) or ALL-1222 (FIGS. 25F-25J) were tested five weeks later (PRE) by anti-CD19 flow cytometry to assess the frequency of leukemic cells in the peripheral blood. Subsequently, mice were treated with: vehicle (FIGS. 25A & 25F), Palbociclib 150 mg/kg once per day (FIGS. 25B & 26G), PROTAC YX-2-107 125 mg/kg (ALL-004) or 150 mg/kg (ALL-1222) once per day (FIGS. 25C & 25H) or PROTAC YX-2-107 twice per day at half dose per injection (FIGS. 25D & 25I) for 10 consecutive days. Then, the percentage of leukemia cells (CD19+) in the peripheral blood was determined at week 7 (POST). (FIGS. 25E & 25J) fold changes of the percentages shown above.

FIGS. 26A-26F show the effect of PROTAC YX-2-107 on peripheral blood leukemia burden of NSG mice injected with a TKI-resistant Ph+ ALL. NSG mice were injected with a primary, human, TKI-resistant (BCR-ABL1T315I) Ph+ ALL sample (#557). (FIGS. 26A-26D) Peripheral blood leukemia burden was analyzed at week 7 post-cell injection (pre) and after 12 and 20 days of treatment with Palbociclib (mixed in the diet to achieve a dose of 150 mg/kg/day) or YX-2-107 IP twice/day at either 25 mg/kg or 50 mg/kg. In these mice, the percentage of peripheral blood leukemic cells (CD19+) was determined at day 14 and 21, respectively. (FIGS. 26E & 26F) fold changes of the percentages shown above.

FIG. 27 shows NLS-CDK4 is resistant to degradation by PROTAC YX-2-107. Immunoblot shows expression of NLS-CDK4 and CDK6 in PROTAC YX-2-107-treated NLS-CDK4-BV173 cells.

### DESCRIPTION OF ILLUSTRATIVE EMBODIMENTS

The present disclosure relates to PROTACs which contain modified linker groups. These compounds show improved property relative those known in the art. In particular, the PROTACs described herein with CDK6 or CDK4 targeting ligands may show one or more advantages of compounds known in the art including but not limited showing improved efficacy, improved selectivity, or show improved bioavailability. Without wishing to be bound by any theory, it is believed that a basic group in the linker may lead to an improvement in molecular properties of the compound and selective degradation for CDK6 over CDK4.

In some embodiments, these compounds may be able to counteract the compensatory increase in CDK6 expression seen with current clinically used CDK4/6 inhibitors (Yang et al., 2017). Additionally, the PROTACs described herein may also have a kinetic advantage over covalent inhibitors since restoration of protein function following PROTAC-induced degradation requires target protein re-synthesis. Furthermore, the PROTACs described herein may be able to exhibit sub-stoichiometric effects by inducing multiple protein ubiquitination events and may overcome potential exposure issues with drugs which require high doses.

### I. ROLE OF CDK IN CANCER CELLS

A. Requirement for MYB in Ph+ ALL Cells

Using a Doxycycline (DOX)-inducible MYB-shRNA (Drabsch et al., 2008), it was shown that silencing MYB expression in Ph+ ALL cell lines (BV173, SUP-B15, Z181) markedly suppresses proliferation, inhibits colony formation, and induces cell cycle arrest (De Dominici et al., 2018). DOX-treated NSG mice injected with shMYB-BV173 cells had a lower leukemia burden and survived much longer than untreated mice (De Dominici et al., 2018)

The effects of MYB silencing were also assessed in mice injected with shMYB primary Ph+ ALL cells. Compared to untreated mice, leukemia load was reduced in DOX-treated mice (De Dominici et al., 2018), but the effect was transient due to the outgrowth of cells with reduced GFP positivity in which MYB expression was not silenced (De Dominici et al., 2018).

B. Identification of MYB Targets in Ph+ ALL Lines.

MYB targets potentially important in Ph+ ALL were identified by microarray analyses of untreated and DOX-treated BV173 and SUP-B15 cells. 79 genes including LEF1, FOXM1, CCND3 (Cyclin D3), CDK6, BCL2, and CDKN1A (p21), showed at least a 1.5-fold change in expression in both lines. Expression of MYB and CDK6 is highly correlated in Ph+ ALL and high-risk childhood ALL. Changes in CDK6, CCDN3 and CDKN1A levels correlate with the cell cycle arrest of MYBsilenced BV173 cells and are biologically significant as indicated by suppressed CDK4/6-dependent RB phosphorylation despite unchanged CDK4 levels.

C. CDK6 but not CDK4 is Required for Ph+ ALL Cell Proliferation.

CDK4 and CDK6 are thought to have redundant roles in the cell cycle and the expression of both isoforms is readily detected in most cases of Ph+ ALL (De Dominici et al., 2018). However, silencing CDK6 alone markedly suppressed proliferation and phospho-RB/FOXM1 expression in Ph+ ALL cells, while silencing CDK4 expression had no effects. This result suggests that in these cells CDK6 exerts a function that is not shared by CDK4. Of interest, CDK6 is predominantly localized in the nucleus of Ph+ ALL cells, while CDK4 appears to be almost exclusively cytoplasmic. This finding could explain the specific requirement for CDK6 by Ph+ ALL cells. Indeed, expression of a nucleus-localized CDK4 rescued the cell cycle arrest of MYB-silenced BV173 cells (De Dominici et al., 2018). CDK4 silencing had no effect on the cell cycle of normal CD34+ hematopoietic progenitors; CDK6 silencing reduced the S phase of CD34+ cells to approximately 60% of that in control cells but the effect was less pronounced than in Palbociclib-treated cells, indicating that CDK4 expression partially compensates for loss of CDK6.

Consistent with these data, RB phosphorylation and FOXM1 levels were completely rescued by CDK6 in CDK4-silenced cells whereas the effect of CDK4, in particular on S807-811 phospho-RB, in CDK6-silenced cells was only partial.

D. Restoring the Expression of CDK6, Cyclin D3 and BCL2 Rescues the Cell Cycle Arrest and Apoptosis Induced by MYB Silencing

Ectopic expression of cyclin D3 did not rescue RB phosphorylation, expression of its target FOXM1 (Anders et al., 2011), or the cell cycle arrest of MYB-silenced BV173 cells. By contrast, expression of CDK6 alone rescued all three of these phenotypes, suggesting that these cells expressed enough cyclin D3 or other cyclin D isoforms to activate CDK6. Co-expression of cyclin D3 and CDK6 was more effective than CDK6 alone, but its growth-promoting effect was transient because it did not rescue the decreased colony formation and apoptosis induced by MYB silencing, possibly because it had no effects on BCL2 levels. Indeed, expression of BCL2 restored in vitro growth, suppressed apoptosis, and partially rescued the clonogenic potential of MYB-silenced cells.

E. Treatment with Palbociclib Markedly Suppresses In Vitro Growth of Ph+ Cells but has a Transient Effect In Vivo

Treatment with Palbociclib (0.5 μM) markedly suppressed viability of BV173 cells and the percentage of S phase (n=8) and colony formation (n=5) of primary Ph+ ALL cells (De Dominici et al., 2018). However, Palbociclib dependent growth inhibition of primary Ph+ ALL in NSG mice was transient with leukemia burden returning to levels seen in untreated mice 30 days after termination of the therapy. This outcome suggests that the duration of the therapy may have been insufficient and that the effect was only cytostatic. Since co-expression of cyclin D3 and CDK6 did not rescue apoptosis and colony formation inhibition induced by MYB silencing, the effect of MYB silencing may be mimicked by the simultaneous targeting of proliferative and anti-apoptotic pathways. Indeed, the Palbociclib/Sabutoclax (BCL2 family antagonist) combination is more effective than either drug used alone ex vivo or in vivo. Since Sabutoclax is a pan-BCL2 inhibitor (Lu et al., 2006) but MYB silencing had no effect on BCL-XL and MCL-1 expression, to mimic MYB silencing more closely the ex vivo effects of Palbociclib in combination with the BCL2 antagonist Venetoclax (Souers et al., 2013) were tested in Ph+ ALL cells. Co-treatment of BV173 and SUP-B15 Ph+ cell lines with Palbociclib and Venetoclax suppressed cell growth more effectively than either drug alone and the effect appears to be synergistic (BV173 cells) or additive (SUP-B15 cells). Interestingly, SUP-B15 cells are much more sensitive than BV173 cells to Venetoclax (nM vs M).

### II. COMPOUNDS OF THE PRESENT DISCLOSURE

The compounds of the present disclosure are shown, for example, below in Table 1, in the summary section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration. In some embodiments, the present compounds may contain two or more atoms which have a defined stereochemical orientation.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

TABLE 1
Compounds of the Present Disclosure

| Com- | |
|---|---|
| pound | |
| ID | Structure |
| YX-2-79 | 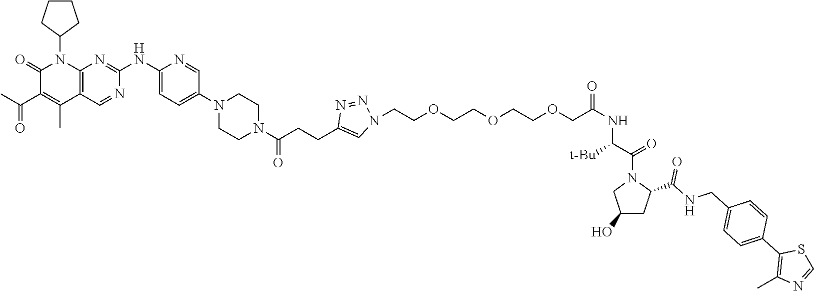 [SMILES: CC(=O)c1c(C)c2cnc(Nc3ccc(N4CCN(C(=O)CCc5cn(CCOCCOCCOCC(=O)N[C@H](C(=O)N6C[C@H](O)C[C@H]6C(=O)NCc6ccc(-c7scnc7C)cc6)C(C)(C)C)nn5)CC4)cn3)nc2n(C2CCCC2)c1=O] |
| YX-2-99 | 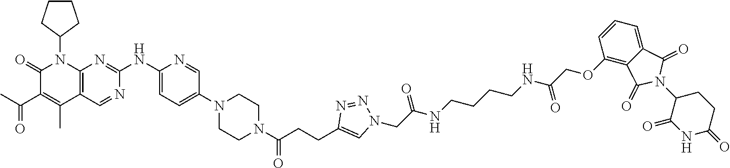 [SMILES: CC(=O)c1c(C)c2cnc(Nc3ccc(N4CCN(C(=O)CCc5cn(CC(=O)NCCCCNC(=O)COc6cccc7c6C(=O)N(C6CCC(=O)NC6=O)C7=O)nn5)CC4)cn3)nc2n(C2CCCC2)c1=O] |
| YX-2-107 | 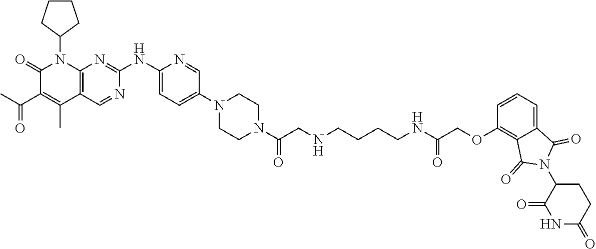 [SMILES: CC(=O)c1c(C)c2cnc(Nc3ccc(N4CCN(C(=O)CNCCCCNC(=O)COc5cccc6c5C(=O)N(C5CCC(=O)NC5=O)C6=O)CC4)cn3)nc2n(C2CCCC2)c1=O] |
| YX-2-196 | 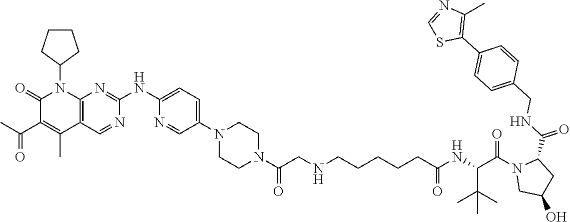 [SMILES: CC(=O)c1c(C)c2cnc(Nc3ccc(N4CCN(C(=O)CNCCCCCC(=O)N[C@H](C(=O)N5C[C@H](O)C[C@H]5C(=O)NCc5ccc(-c6scnc6C)cc5)C(C)(C)C)CC4)cn3)nc2n(C2CCCC2)c1=O] |
| YX-2-233 | 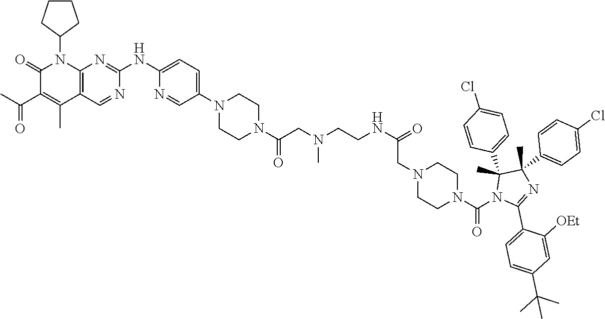 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCN(C)CC(=O)N2CCN(c3ccc(Nc4ncc5c(C)c(C(C)=O)c(=O)n(C6CCCC6)c5n4)nc3)CC2)CC1] |
| YX-2-238 | 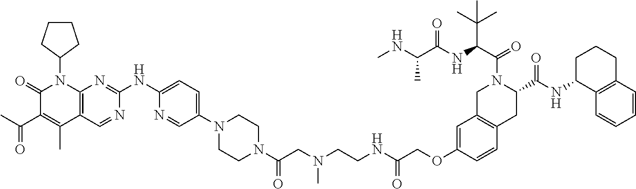 [SMILES: CN[C@@H](C)C(=O)N[C@H](C(=O)N1Cc2cc(OCC(=O)NCCN(C)CC(=O)N3CCN(c4ccc(Nc5ncc6c(C)c(C(C)=O)c(=O)n(C7CCCC7)c6n5)nc4)CC3)ccc2C[C@H]1C(=O)N[C@@H]1CCCc2ccccc21)C(C)(C)C] |
| AC-1-027 | 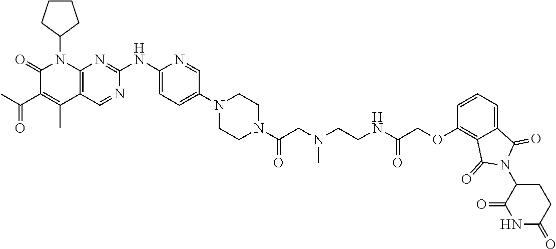 [SMILES: CC(=O)c1c(C)c2cnc(Nc3ccc(N4CCN(C(=O)CN(C)CCNC(=O)COc5cccc6c5C(=O)N(C5CCC(=O)NC5=O)C6=O)CC4)cn3)nc2n(C2CCCC2)c1=O] |
| AC-1-079 | 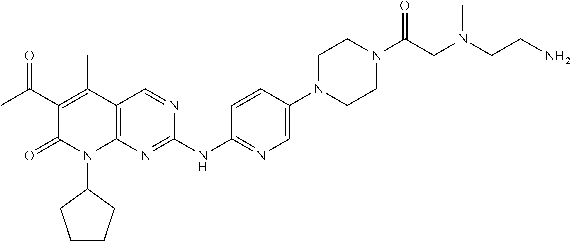 [SMILES: CC(=O)c1c(C)c2cnc(Nc3ccc(N4CCN(C(=O)CN(C)CCN)CC4)cn3)nc2n(C2CCCC2)c1=O] |
| AC-1-091 | 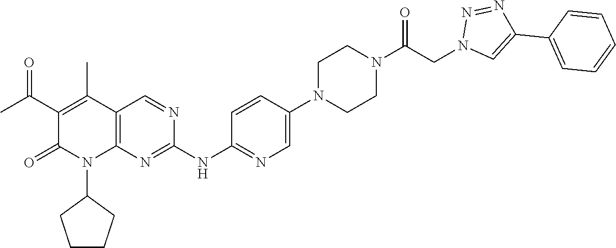 [SMILES: CC(=O)c1c(C)c2cnc(Nc3ccc(N4CCN(C(=O)Cn5cc(-c6ccccc6)nn5)CC4)cn3)nc2n(C2CCCC2)c1=O] |
| AC-1-112 | 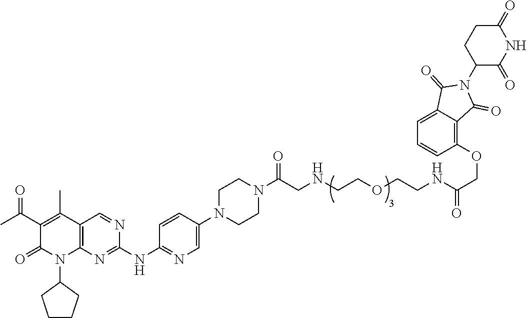 |
| AC-1-212 | 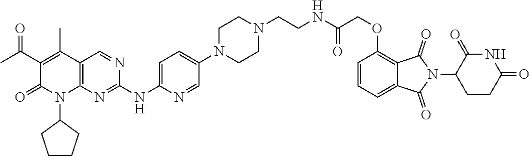 [SMILES: CC(=O)c1c(C)c2cnc(Nc3ccc(N4CCN(CCNC(=O)COc5cccc6c5C(=O)N(C5CCC(=O)NC5=O)C6=O)CC4)cn3)nc2n(C2CCCC2)c1=O] |
| AC-1-277 | 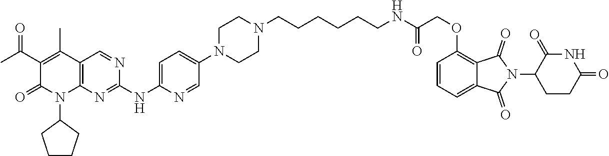 [SMILES: CC(=O)c1c(C)c2cnc(Nc3ccc(N4CCN(CCCCCCNC(=O)COc5cccc6c5C(=O)N(C5CCC(=O)NC5=O)C6=O)CC4)cn3)nc2n(C2CCCC2)c1=O] |
| AC-2-011 | 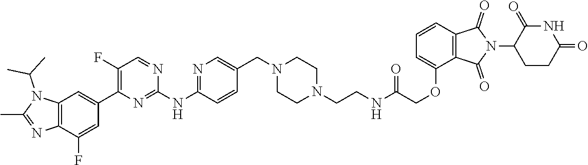 [SMILES: Cc1nc2c(F)cc(-c3nc(Nc4ccc(CN5CCN(CCNC(=O)COc6cccc7c6C(=O)N(C6CCC(=O)NC6=O)C7=O)CC5)cn4)ncc3F)cc2n1C(C)C] |
| YX-2-115 | 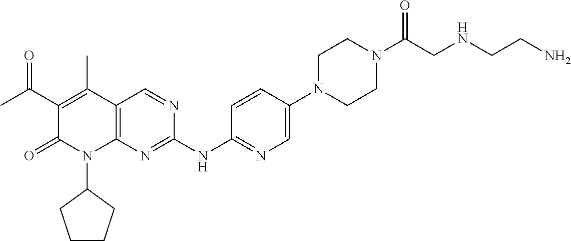 [SMILES: CC(=O)c1c(C)c2cnc(Nc3ccc(N4CCN(C(=O)CNCCN)CC4)cn3)nc2n(C2CCCC2)c1=O] |

### III. PHARMACEUTICAL FORMULATIONS AND ROUTES OF ADMINISTRATION

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the patient has eaten or will eat.

### IV. CHEMICAL DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof, in a divalent context “phosphate” means —OP(O)(OH)O— or a deprotonated form thereof, “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

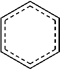

covers, for example,

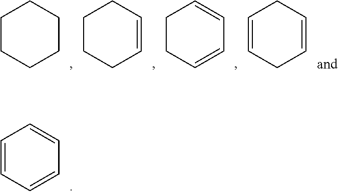

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g. [symbol] for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” g e single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

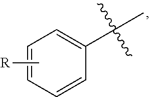

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

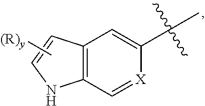

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}>” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

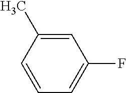

is also taken to refer to

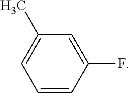

Aromatic compounds may also be depicted using a circle to represent the delocalized nature of the electrons in the fully conjugated cyclic π system, two non-limiting examples of which are shown below:

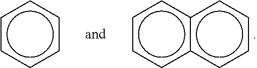

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ⁱBu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

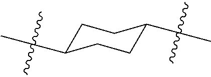 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

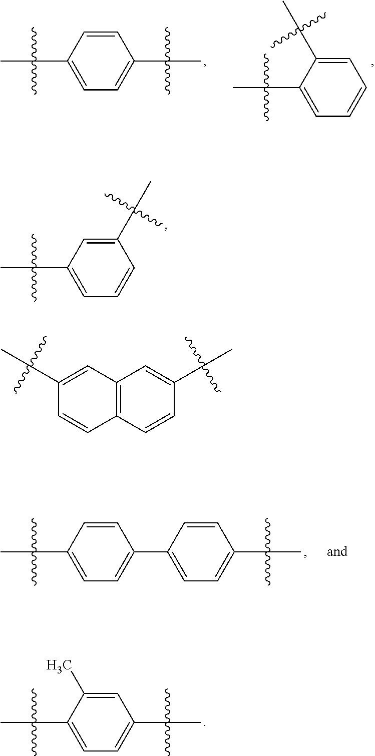

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. The term “heteroarenediyl” refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, oxygen or sulfur, and wherein the divalent group consists of no atoms other than carbon, hydrogen, aromatic nitrogen, aromatic oxygen and aromatic sulfur. If more than one ring is present, the rings are fused; however, the term heteroarenediyl does not preclude the presence of one or more alkyl or aryl groups (carbon number limitation permitting) attached to one or more ring atoms. Non-limiting examples of heteroarenediyl groups include:

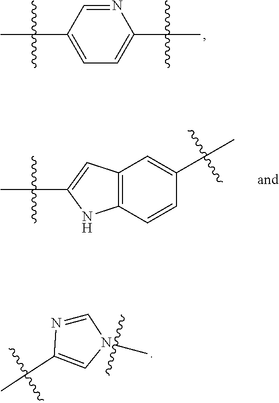

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. A “heterocycloalkane” refers to the class of compounds having the formula H—R, wherein R is heterocycloalkyl.

The term “heterocycloalkanediyl” refers to a divalent cyclic group, with two carbon atoms, two nitrogen atoms, or one carbon atom and one nitrogen atom as the two points of attachment, said atoms forming part of one or more ring structure(s) wherein at least one of the ring atoms of the non-aromatic ring structure(s) is nitrogen, oxygen or sulfur, and wherein the divalent group consists of no atoms other than carbon, hydrogen, nitrogen, oxygen and sulfur. If more than one ring is present, the rings are fused. As used herein, the term heterocycloalkanediyl does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkanediyl groups include:

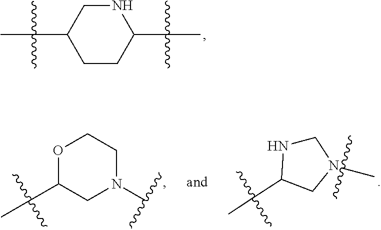

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### V. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—CDK4/6-Inhibiting PROTACs Synthesis and Characterization

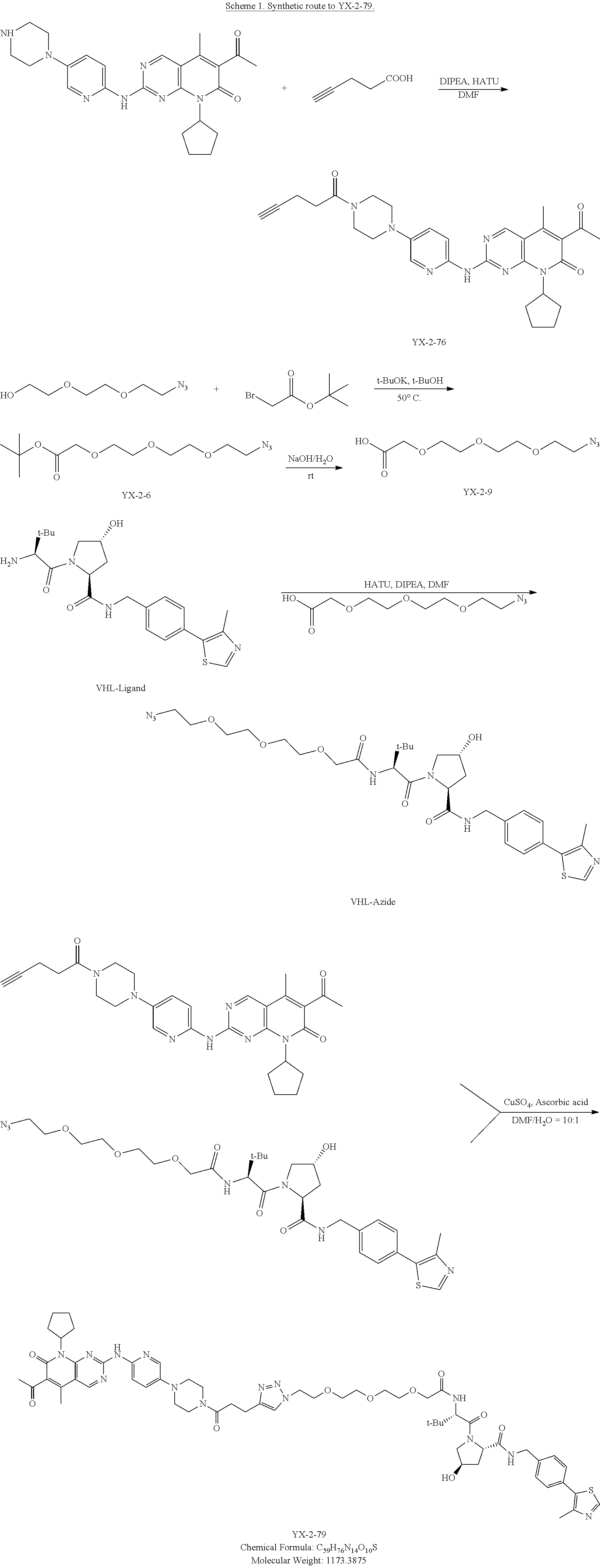

A mixture of palbociclib (224 mg, 0.5 mmol) and but-3-ynoic acid (50 mg, 0.5 mmol) in DMF (10 ml) was treated with DIPEA (166 μL, 1.0 mmol) and then HATU (190 mg, 0.5 mmol) and stirred at ambient temperature for 1 hour. After which time the reaction mixture was diluted with H₂O and extracted with EtOAc. The organic layer was dried by Na₂SO₄ and concentrated in vacuum. The resulting material was purified by chromatography on silica to afford palbociclib-alkyne YX-2-76 (185 mg, 70% yield). LC-MS RT=1.43, ES+ve 528.

2-(2-(2-azidoethoxy)ethoxy)ethanol (3.0 g, 16.9 mmol) was dissolved in t-BuOH (30 ml), and then KOt-Bu (3.9 g, 33.8 mmol) was added into the reaction at ambient temperature. After 1 hour, tert-butyl 2-bromoacetate (6.6 g, 33.8 mmol) was added slowly to the reaction mixture and stirred at 50° C. for 5 hours. The reaction solvent was removed and diluted with H₂O and extracted with EtOAc. The organic layer was dried by Na₂SO₄ and concentrated in vacuum. The resulting material was purified by chromatography on silica to afford palbociclib-alkyne YX-2-6 (3.0 g, 75% yield). Then the product was treated with dioxane and concentrated HCl, stirred at room temperature for 2 hours, solvent was removed and used it directly for next step.

A mixture of VHL-ligand (430 mg, 1.0 mmol) and YX-2-9 (233 mg, 1.0 mmol) in DMF (10 ml) was treated with DIPEA (360 μL, 2.0 mmol) and then HATU (380 mg, 1.0 mmol) and stirred at ambient temperature for 2 hours. After which time the reaction mixture was diluted with H₂O and extracted with EtOAc. The organic layer was dried by Na₂SO₄ and concentrated in vacuum. The resulting material was purified by chromatography on silica to afford VHL-Azide (529 mg, 82% yield).

CuSO₄ (5 mg, 0.03 mmol) and ascorbic acid (5 mg, 0.03 mmol) was added to a solution of palbociclib-alkyne YX-2-76 (52 mg, 0.1 mmol) with VHL-azide (62 mg, 0.1 mmol) in DMF/H₂O (4 mL:0.4 mL) and stirred at ambient temperature for 24 hour. The mixture was diluted with H₂O and extracted with EtOAc. The organic layer was dried by Na₂SO₄ and concentrated in vacuum. The resulting material was purified by chromatography on silica to afford YX-2-79 (24 mg, 20% yield). ¹H NMR (400 MHz, DMSO) δ 10.13 (s, 1H), 8.96 (d, J=6.8 Hz, 2H), 8.58 (s, 1H), 8.07 (s, 1H), 7.88 (d, J=9.2 Hz, 1H), 7.81 (s, 1H), 7.49 (d, J=6.8 Hz, 1H), 7.45-7.33 (m, 4H), 5.88-5.77 (m, 1H), 5.14 (s, 1H), 4.57 (d, J=9.4 Hz, 1H), 4.45 (d, J=5.9 Hz, 2H), 4.35 (s, 2H), 4.31-4.20 (m, 2H), 3.96 (s, 2H), 3.77 (d, J=5.1 Hz, 2H), 3.60 (d, J=5.0 Hz, 7H), 3.52 (s, 4H), 3.12 (s, 3H), 2.86 (d, J=7.3 Hz, 2H), 2.71 (t, J=14.2 Hz, 3H), 2.43 (d, J=4.3 Hz, 4H), 2.31 (s, 3H), 2.24 (s, 2H), 2.05 (s, 2H), 1.90 (s, 3H), 1.77 (s, 2H), 1.59 (s, 3H), 1.24 (s, 3H), 0.94 (s, 7H), 0.85 (d, J=10.5 Hz, 2H). LC-MS RT=1.42, [(M+2H)/2]=588.

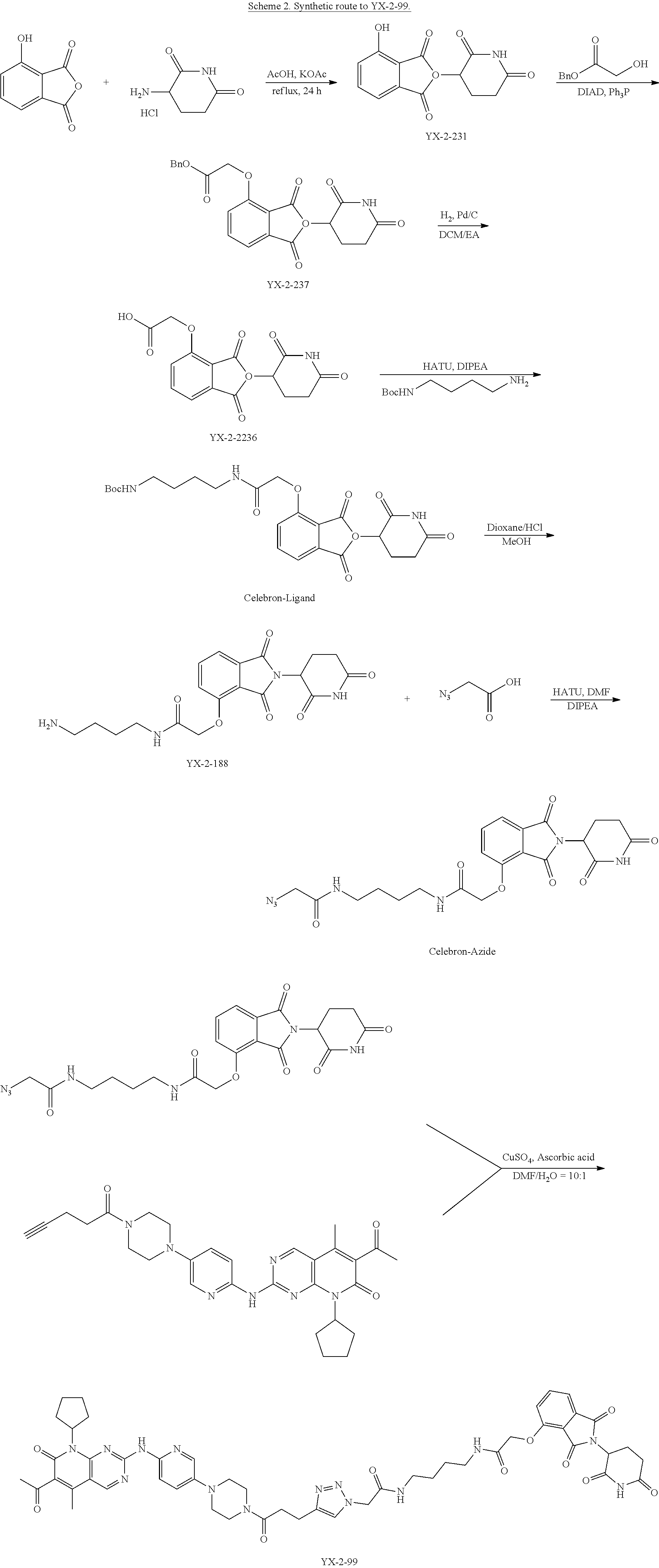

Celebron-ligand (502 mg, 1.0 mmol) was dissolved in MeOH (8 Ml) and treated with dioxane/HCl (4.0 M, 1.0 mL). After stirring at room temperature for 2 hours, the reaction solvent was removed, and the crude product was used directly in next step. A mixture of the amine and 2-azidoacetic acid (100 mg, 1.0 mmol) in DMF (10 mL) was treated with DIPEA (360 μL, 2.0 mmol) and then HATU (380 mg, 1.0 mmol) and stirred at ambient temperature for 2 hours. The reaction mixture was then diluted with H₂O and extracted with EtOAc. The organic layer was dried with Na₂SO₄ and concentrated under vacuum. The resulting material was purified by chromatography on silica to afford Celebron-Azide (412 mg, 85% yield).

A mixture of palbociclib-alkyne YX-2-76 (26 mg, 0.05 mmol) and Celebron-azide (24 mg, 0.05 mmol) in DMF/H₂O (2 mL: 0.2 mL) was treated with CuSO₄ (2.5 mg, 0.015 mmol), then ascorbic acid (2.5 mg, 0.015 mmol) was added before stirring at ambient temperature for 24 hours. The reaction mixture was then diluted with H₂O and extracted with EtOAc. The organic layer was dried with Na₂SO₄ and concentrated under vacuum. The resulting material was purified by chromatography on silica to afford YX-2-99 (12 mg, 24% yield). ¹H NMR (400 MHz, DMSO) δ 11.11 (s, 1H), 10.12 (s, 1H), 8.96 (s, 1H), 8.28 (s, 1H), 8.07 (s, 1H), 7.97 (s, 1H), 7.88 (d, J=8.8 Hz, 1H), 7.79 (d, J=8.7 Hz, 1H), 7.49 (d, J=7.1 Hz, 1H), 7.38 (d, J=8.2 Hz, 1H), 5.83 (s, 1H), 5.12 (d, J=7.1 Hz, 1H), 5.00 (s, 1H), 4.77 (s, 1H), 3.62 (s, 3H), 3.16 (s, 4H), 2.88 (s, 2H), 2.74 (s, 2H), 2.54 (s, 3H), 2.42 (s, 1H), 2.31 (s, 2H), 2.26 (s, 1H), 2.01 (s, 2H), 1.89 (s, 1H), 1.78 (s, 2H), 1.59 (s, 2H), 1.44 (s, 2H), 1.23 (s, 3H), 0.85 (s, 1H). LC-MS RT=1.30, ES+ve 1014.

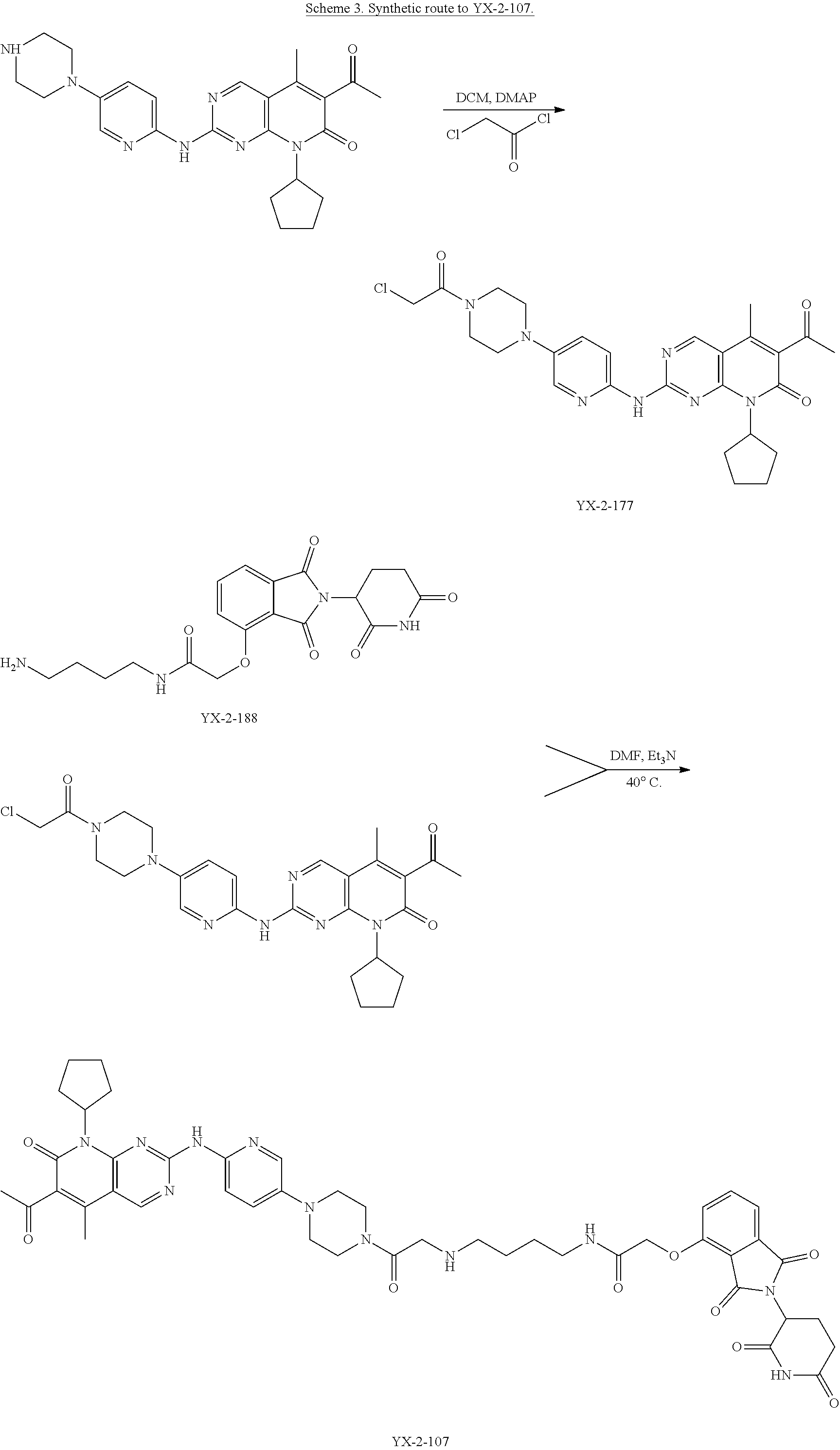 [SMILES: CC(=O)c1c(C)c2cnc(Nc3ccc(N4CCNCC4)cn3)nc2n(C2CCCC2)c1=O ; O=C(Cl)CCl ; CC(=O)c1c(C)c2cnc(Nc3ccc(N4CCN(C(=O)CCl)CC4)cn3)nc2n(C2CCCC2)c1=O ; NCCCCNC(=O)COc1cccc2c1C(=O)N(C1CCC(=O)NC1=O)C2=O ; CC(=O)c1c(C)c2cnc(Nc3ccc(N4CCN(C(=O)CCl)CC4)cn3)nc2n(C2CCCC2)c1=O ; CC(=O)c1c(C)c2cnc(Nc3ccc(N4CCN(C(=O)CNCCCCNC(=O)COc5cccc6c5C(=O)N(C5CCC(=O)NC5=O)C6=O)CC4)cn3)nc2n(C2CCCC2)c1=O]

Chloroacetyl chloride (144 μL, 1.2 mmol) was added to a mixture of palbociclib (448 mg, 1.0 mmol) and DMAP (catalytic amount) in dry dichloromethane at 0° C. The reaction mixture was stirred at room temperature for 3 hours. Dichloromethane was removed, then ethyl acetate was added to the mixture. The suspension was filtered and the solid was washed with ethyl acetate and vacuum dried to afford YX-2-177 (498 mg, 95% yield). LC-MS RT=1.42, ES+ve 524.

Palbociclib-chloride YX-2-177 (52 mg, 0.1 mmol) and Celebron-amine YX-2-188 (40 mg, 0.1 mmol) were added to DMF (5 mL) and then treated with triethylamine (41 μL, 0.3 mmol). The mixture was heated to 40° C. for overnight. Upon completion, the solvent was removed and the crude product was subjected directly to purification by chromatography on silica to afford the YX-2-107 (9.0 mg, 10% yield). ¹H NMR (400 MHz, DMSO) δ 10.15 (s, 1H), 8.96 (s, 1H), 8.09 (s, 2H), 7.94-7.69 (m, 2H), 7.59-7.45 (m, 2H), 7.41 (d, J=8.3 Hz, 1H), 5.90-5.77 (m, 1H), 5.13 (dd, J=12.8, 5.4 Hz, 1H), 4.79 (s, 2H), 3.87 (s, 2H), 3.64 (dd, J=42.3, 24.1 Hz, 5H), 3.18 (d, J=13.6 Hz, 7H), 2.98-2.82 (m, 2H), 2.78 (s, 2H), 2.60 (dd, J=32.9, 17.5 Hz, 3H), 2.42 (s, 3H), 2.31 (s, 3H), 2.24 (s, 2H), 2.04 (s, 2H), 1.88 (s, 2H), 1.76 (s, 3H), 1.59 (s, 4H), 1.49 (s, 3H), 1.24 (s, 3H), 0.84 (dd, J=10.8, 6.8 Hz, 2H). LC-MS RT=1.19, ES+ve 890. (AC-1-027 was prepared using an analogous route).

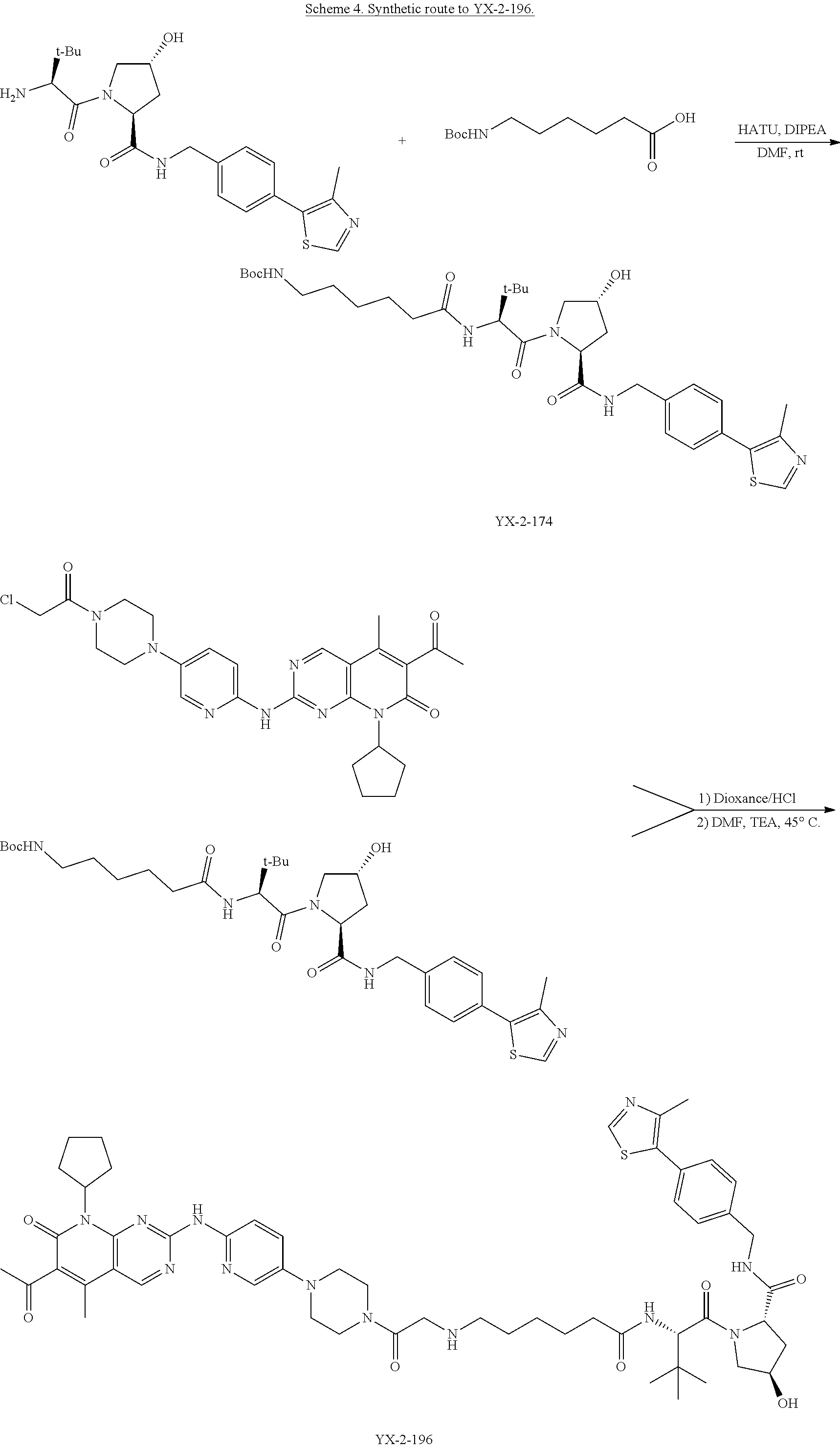

A mixture of VHL ligand (934 mg, 2.0 mmol) and 6-(tert-butoxycarbonylamino)hexanoic acid (508 mg, 2.2 mmol) in DMF (15 mL) was treated with DIPEA (720 μL, 4.0 mmol). HATU (769 mg, 2.0 mmol) was added and the reaction mixture stirred at ambient temperature for 1 hour. The reaction mixture was then diluted with H₂O and extracted with EtOAc. The organic layer was dried by Na₂SO₄ and concentrated under vacuum. The resulting material was purified by chromatography on silica to afford YX-2-174 (610 mg, 48% yield). LC-MS RT=1.60, ES+ve 645.

The above compound was dissolved in DCM (10 mL) and treated with TFA (2 mL), stirred for 2 hours, and then the solvent was removed. The crude product (61 mg, 0.095 mmol) and YX-2-177 (49.7 mg, 0.095 mmol) were added to DMF (12 mL) and treated with triethylamine (38 μL, 0.3 mmol). The mixture was heated to 45° C. for overnight. Upon completion, the solvent was removed and the crude product was subjected directly to purification by chromatography on silica to afford the title compound YX-2-196 (3.6 mg, 4% yield). LC-MS RT=1.36, ES+ve 1031.

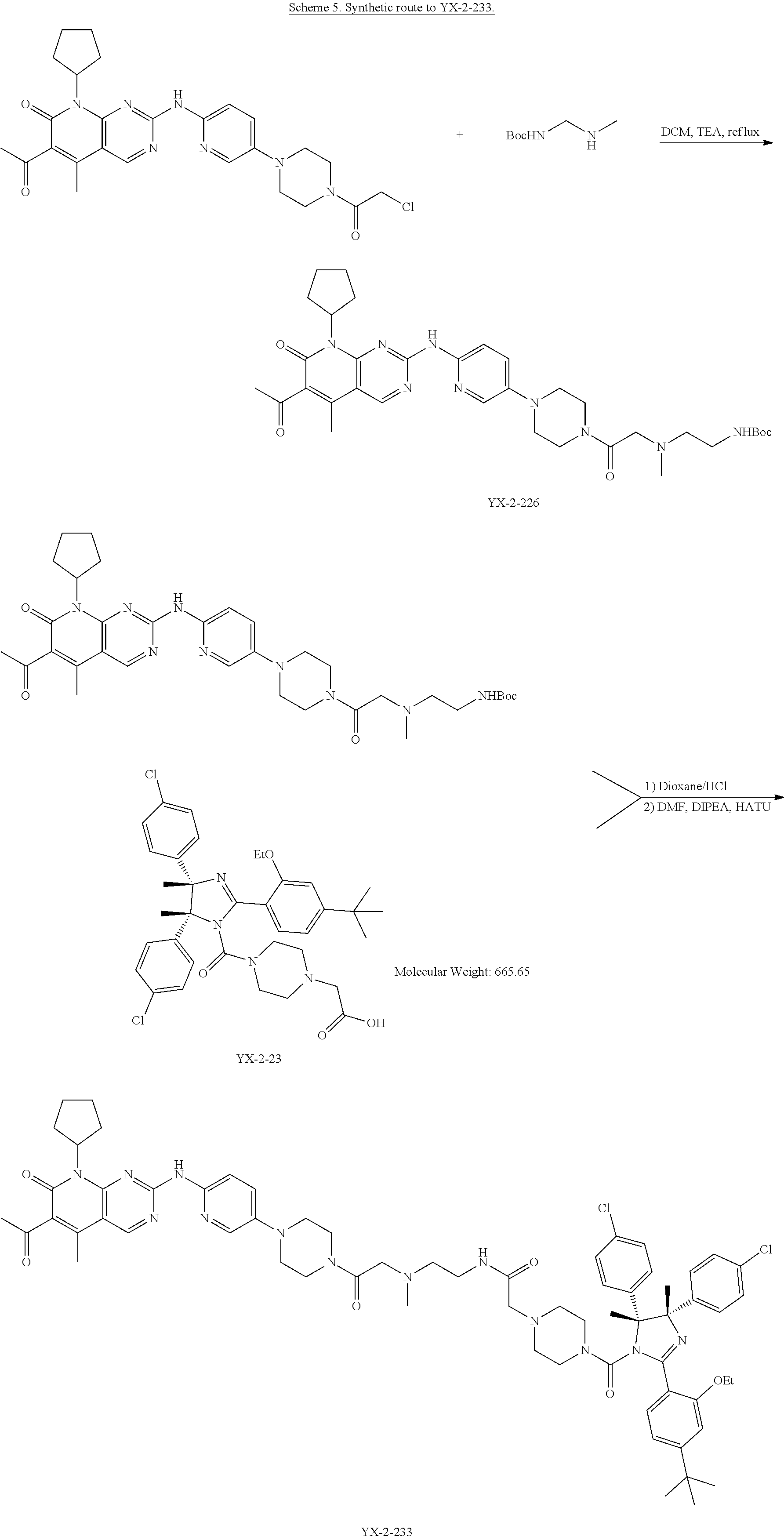

A mixture of YX-2-177 (100 mg, 0.2 mmol) and tert-butyl (methylamino)methylcarbamate (34 mg, 0.4 mmol) in DCM (8 mL) was treated with TEA (100 μl, 0.8 mmol) and then refluxed for overnight. DCM was evaporated and the crude product was purified by chromatography on silica to afford YX-2-226 (40 mg, 30% yield). LC-MS RT=1.32, ES+ve 662.

YX-2-226 (20 mg, 0.03 mmol) was dissolved in DCM (1 mL) and treated with TFA (0.1 mL), stirred for 2 hours. Next, the reaction solvent was removed and the crude product and YX-2-23 were dissolved in DMF (3 mL) and treated with DIPEA (10 μl, 0.06 mmol) and HATU (12 mg, 0.03 mmol). The mixture was stirred at room temperature for 2 hours. Upon completion, the mixture was treated with ethyl acetate and water. The organic phase was separated and evaporated to dryness and the product was purified by chromatography on silica to afford YX-2-233 (6 mg, 17% yield). ¹H NMR (400 MHz, CD₃OD_SPE) δ 9.10 (s, 1H), 8.17 (d, J=9.6 Hz, 1H), 7.91 (s, 1H), 7.78 (d, J=7.9 Hz, 1H), 7.58 (d, J=9.5 Hz, 1H), 7.43-7.30 (m, 2H), 7.12 (dd, J=37.4, 16.5 Hz, 5H), 6.09-5.90 (m, 1H), 4.40 (s, 2H), 4.24 (s, 1H), 3.81 (s, 2H), 3.63 (s, 3H), 3.52-3.34 (m, 5H), 3.20 (s, 1H), 3.05 (d, J=15.2 Hz, 3H), 2.77 (s, 1H), 2.61 (s, 1H), 2.50 (s, 2H), 2.43 (s, 2H), 2.31 (s, 2H), 2.11 (d, J=16.6 Hz, 3H), 2.03 (s, 2H), 1.90 (s, 2H), 1.70 (s, 3H), 1.48 (d, J=7.0 Hz, 2H), 1.45 (s, 6H), 1.29 (s, 5H), 0.88 (dd, J=15.7, 7.7 Hz, 3H). LC-MS RT=1.66, [(M+2H)/2]=605.

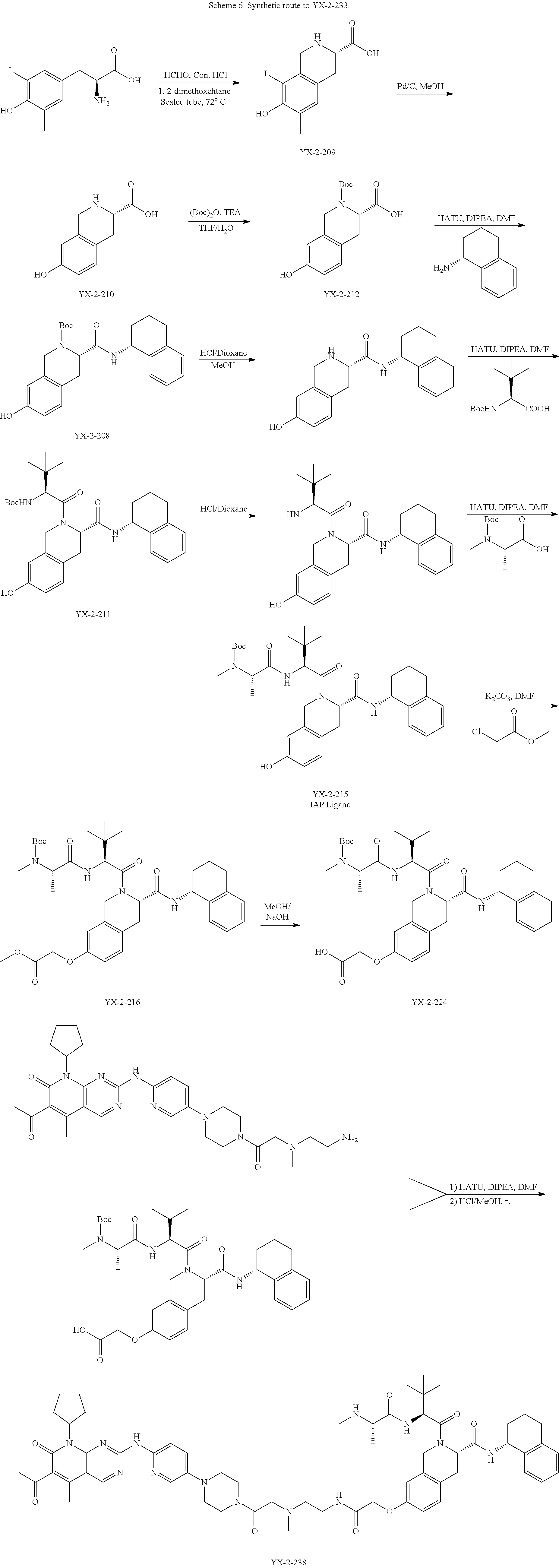

A solution of IAP ligand (80 mg, 0.13 mmol) and methyl 2-chloroacetate (40 μL, 0.52 mmol) in DMF (4 mL) was treated with potassium carbonate (50 mg, 0.52 mmol) and the mixture stirred at room temperature overnight. The crude product was subjected directly to purification by chromatography on silica to afford YX-2-216 (40 mg, 44% yield). YX-2-216 (230 mg, 0.33 mmol) was dissolved in MeOH (2 mL) and treated with NaOH/H₂O (100 mg/2 mL) solution, stirred at room temperature for 2 hours, and then 1N HCl was added to the reaction mixture to adjust the pH to 3-4. The mixture was then treated with ethyl acetate and water, the organic phase separated and evaporated to dryness, and the crude product purified by chromatography on silica to afford YX-2-224 (160 mg, 73% yield). LC-MS RT=1.95, ES+ve 679.

YX-2-226 (20 mg, 0.03 mmol) was dissolved in DCM (1 mL) and treated with TFA (0.1 mL) before being stirred at room temperature for 2 hours. The solvent was then removed, and the crude product was used directly for next step. A mixture of the crude product and YX-2-224 (20 mg, 0.025 mmol) in DMF was treated with DIPEA (20 μL, 0.05 mmol) and then HATU (12 mg, 0.025 mmol) and the mixture was stirred at ambient temperature for 1 hour. The reaction mixture was then diluted with H₂O and extracted with EtOAc. The organic layer was dried with Na₂SO₄ and concentrated under vacuum. The crude product was treated with dioxane/HCl (4.0 M, 0.05 mL) and the mixture stirred for 2 hours. The solvent was then removed and the crude product purified directly by reverse-HPLC to afford YX-2-238 (10 mg, 36% yield). LC-MS RT=1.60, [(M+2H)/2]=562.

### Example 2—Biological Data

It was envisioned that a potent CDK6 kinase inhibitor tethered to an E3 ligase-recruiting molecule may bind to CDK6 and degrade this protein, potentially providing more specific and durable inhibition than currently possible with small molecule inhibitors. Design of an initial CDK4/6-targeted-PROTAC was guided by the X-ray crystal structure of palbociclib in complex with CDK6 (PDB code: 2euf) (Lu et al., 2006). The site of linker attachment is important to maintain ligand affinity. The piperazine tail of palbociclib protrudes from the active site (ATP pocket) towards the solvent suggesting that it may tolerate linker attachment to various E3 ligase-recruiting molecules (Wang et al., 2016). Synthesis of several compounds to understand what can be tolerated were undertaken. The CRBN E3-Amine (Scheme 7) was synthesized using a slight modification of the published procedure (Winter et al., 2015). Palbociclib was reacted with chloroacetyl chloride followed by alkylation with CRBN E3-Amine to yield the palbociclib-PROTAC, YX-2-107 (Schemes 3 and 7). Control compound YX-2-115 was also synthesized (Scheme 7; compound 4d described in Wang et al., 2016).

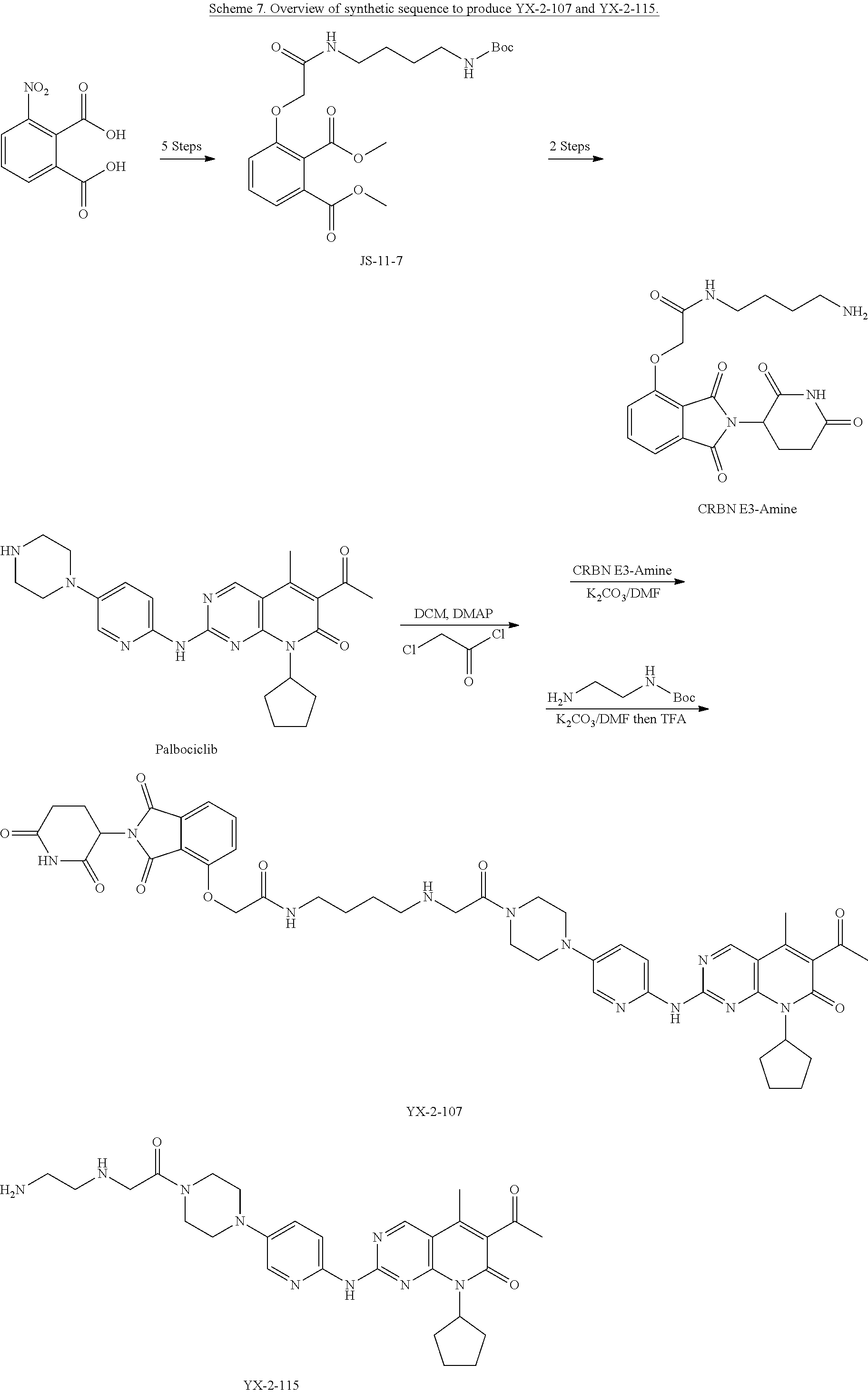

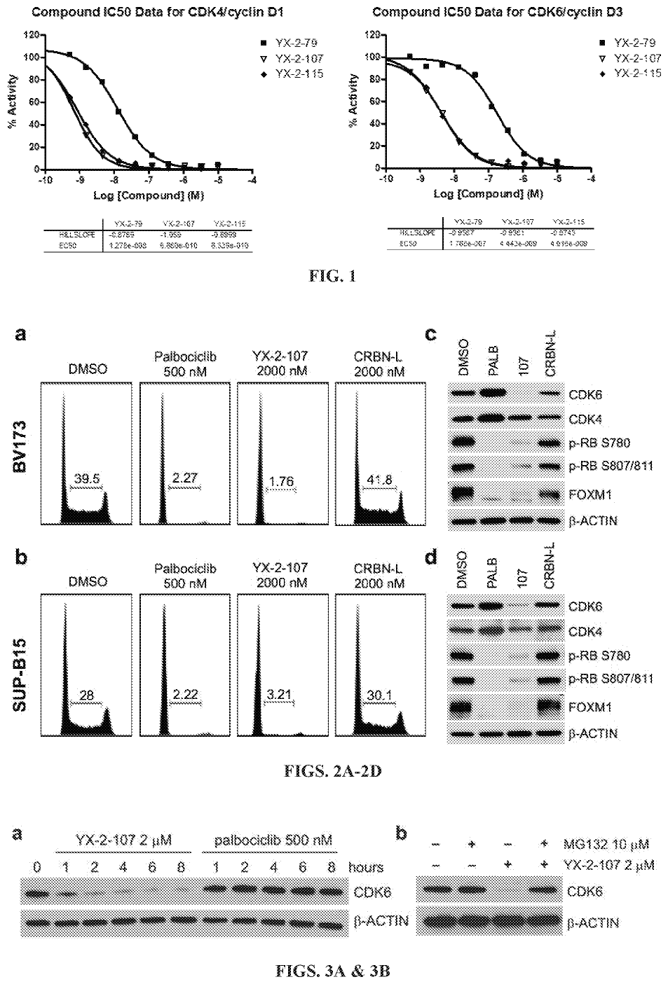

Three palbociclib derivatives, YX-2-79 (Scheme 1), YX-2-107, and YX-2-115, were evaluated for their ability to inhibit CDK4 and CDK6 kinase activity. Compound YX-2-115 and the PROTAC YX-2-107 potently inhibit CDK6 and CDK4. YX-2-79 is 40-fold less potent (FIG. 1 and Table 2). Next, the effects of YX-2-107 were tested in Ph+BV173 and SUP-B15 cells and found that it inhibits S phase entry, RB phosphorylation, and FOXM1 expression but at a higher concentration than palbociclib (FIGS. 2A-2D).

TABLE 2
Inhibition of CDK4 and CDK6 Kinase Activity
for YX-2-79, YX-2-107, and YX-2-115.
Compound IC50 (nM)

| | YX-2-79 | YX-2-107 | YX-2-115 | Staurosporine |
|---|---|---|---|---|
| Kinases | IC50 nM | IC50 nM | IC50 nM | IC50 nM |
| CDK4/cyclinD1 | 12.78 | 0.69 | 0.83 | 8.93 |
| CDK6/cyclinD3 | 176.80 | 4.44 | 4.02 | 12.28 |

A key finding was that palbociclib treatment induced increased expression of CDK4 and, especially, CDK6; by contrast, CDK4 expression did not increase in YX-2-107-treated cells and levels of CDK6 were markedly decreased (FIGS. 2A-2D). A control compound consisting of the cereblon ligand and the linker (CRBN-L) had no effect (FIGS. 2A-2D). Downregulation of CDK6 in Ph+BV173 cells was detected as early as one hour post-treatment with YX-2-107 and at 4 hours, levels of CDK6 were markedly suppressed (FIG. 3A). However, treatment with the proteasome inhibitor MG132 restored CDK6 expression at the levels of untreated cells (FIG. 3B), strongly suggesting that in YX-2-107-treated Ph+ ALL cells CDK6 is targeted for proteasome degradation via cereblon-dependent ubiquitination. In an in vivo study, NSG mice (three/group) were injected with primary Ph+ ALL cells, and treated (3 consecutive days) with palbociclib or YX-2-107, or left untreated when peripheral blood CD19+ cells reached >50%; then, bone marrow cells (>90% CD19+/CD10+) were purified and assessed for cell cycle activity and phospho-RB, FOXM1, and CDK4/CDK6 levels. Palbociclib and YX-2-107 were indistinguishable in suppressing S phase cells and abolishing RB phosphorylation and FOXM1 expression (FIGS. 4A-4C). However, treatment with YX-2-107 suppressed CDK4 and CDK6 levels while CDK6 expression was instead upregulated by palbociclib. To expand the platform of CDK4/6 targeted PROTACs, PROTAC YX-2-233 (FIG. 5A) was synthesized by linking the palbociclib derivative YX-2-115 (Scheme 7) to the MDM2 antagonist RG7112 (Tovar et al., 2013), to recruit the MDM2 E3 ligase. Ex vivo experiments using the BV173 and the SUP-B15 Ph+ ALL cell lines show that treatment with YX-2-233 induced a marked decrease in the number of S phase cells and in RB phoshorylation (FIGS. 5B & 5C). In contrast to YX-2-107, treatment with YX-2-233 induced a marked decrease in CDK4 and CDK6 levels (FIG. 5C).

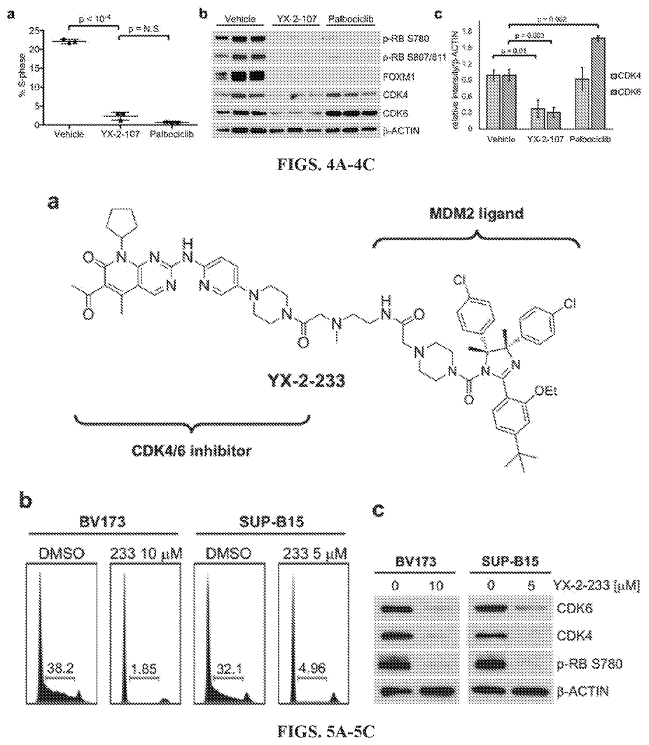

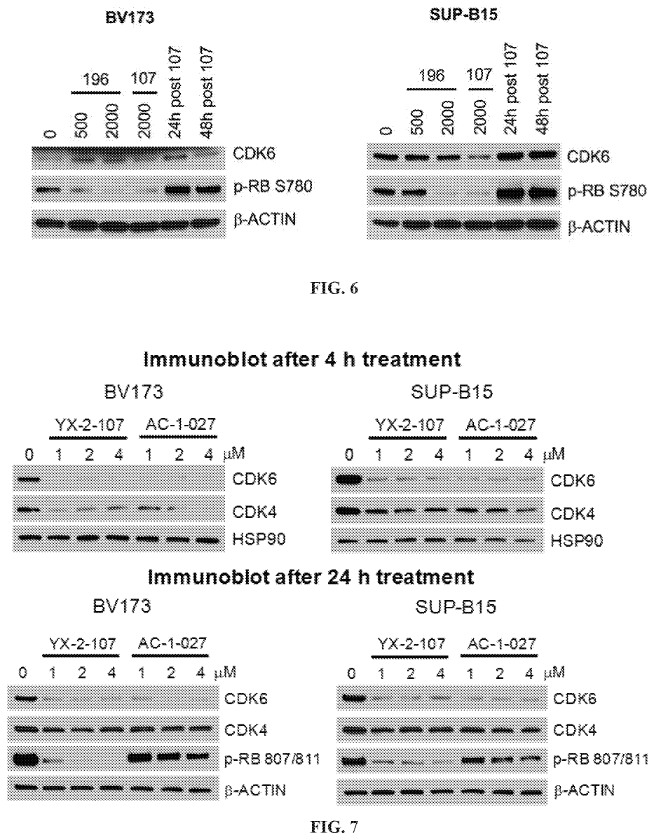

Additional PROTACs were developed to optimize binding affinity and cellular potency by combining CDK4/6 binding molecules, tethers of different length and hydrophobicity, and utilizing various E3 ligase recruiting molecules. Analogs closely related to YX-2-107 that maintain kinase inhibitor potency were synthesized as a surrogate to measuring binding affinity directly. Assays were developed to evaluate the binding affinity of the PROTAC derivatives to their targeted E3 ligase. YX-2-233 (FIG. 5A) targets the least 3-4 additional CDK6 inhibitors (other than palbociclib) taking advantage of the structural requirements for inhibiting CDK6 (Lu et al., 2006) and use these as ligands to conjugate with E3 ligase recruiting molecules. Optimization of the E3 ligase recruiting molecule using binding assays may improve ternary complex formation (Wurz et al., 2018 and Galdeano et al., 2014). E3 ligase recruiting molecules include cIAP ligands and VHL ligands (Sato et al., 2008, Lai and Crews, 2017, and Burslem and Crews, 2017). The VHL E3 ligase recruiting ligand (Galdeano et al., 2014, Galdeano et al., 2012, and Matyskiela et al., 2018) is used to recruit the von Hippel-Lindau protein (pVHL), the substrate recognition subunit of the VHL E3 ligase that targets HIF-1a for degradation. A VHL-recruiting ligand PROTAC was synthesized, YX-2-196 (Scheme 4), and exhibited similar inhibition of phospho-RB (FIG. 6). The difference in decreasing CDK6 levels may be a result of CDK6 not being as efficiently ubiquitinated with YX-2-196 in contrast to YX-2-107. A cIAP recruiting ligand redirects the function of the E3 ligase cIAP, which normally degrades caspase proteins, to ubiquitinate and degrade CDK6. A cIAP-containing compound, AC-1-027, was synthesized. A comparison of the effects of YX-2-107 and AC-1-027 is shown in FIG. 7. Various tethers are tolerated, including alkyl and PEG linkers, and shorter and longer linkers. Tethers constructed with an amide bond or a triazole (Wurz et al., 2018) were also prepared to demonstrate compatibility of components. Further SAR for potency against the target, potency for recruiting the E3 ligase, and molecular properties are planned. Optimized molecules, although typically larger than normal drugs, are cell permeable and have adequate metabolic stability as demonstrated by synthesizing compounds that are effective in vivo. PROTACs may provide an additional layer of selectivity relative to a competitive inhibitor (Lai and Crews, 2017). This effect is seen empirically through the preferential degradation of CDK6 over CDK4.

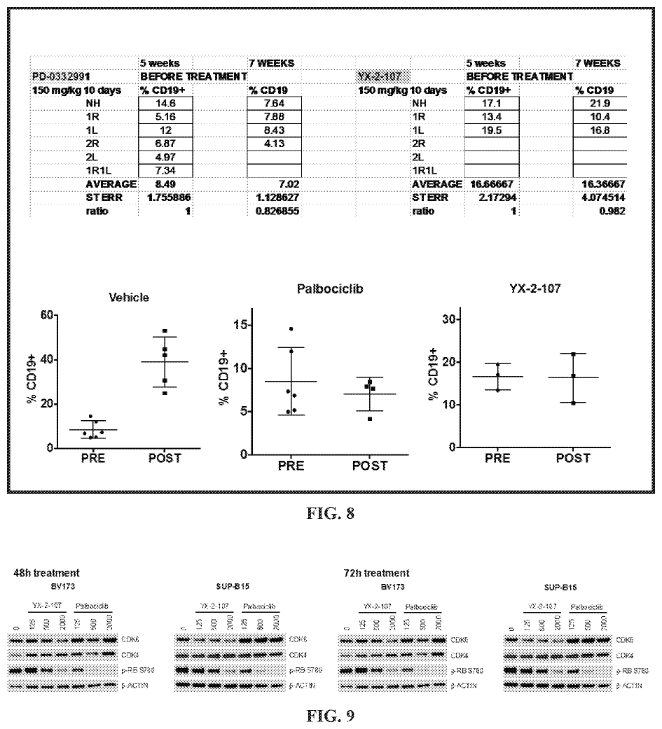

An in vivo experiment to compare the effects on leukemia load post 10 days treatment with daily IP injections of palbociclib and YX-2-107 is shown in FIG. 8. YX-2-107 is effective in blocking leukemia growth and there were no side effects noted. The effect is comparable to palbociclib. It is noted that i) the leukemia load at the starting of the treatment was higher in the YX-2-107 group as compared to the palbociclib group; and ii) 150 mg/kg of palbociclib was employed compared to 125 mg/kg for YX-2-107. The actual concentration of YX-2-107 was even lower considering that the M.W. of palbociclib is lower than YX-2-107.

Additional effects of YX-2-107 in Ph+BV173 and SUP-B15 cells is shown in FIG. 9. YX-2-107 inhibits RB phosphorylation and reduces CDK6 protein levels selectively over CDK4. YX-2-107 is slightly weaker compared to palbociclib but has the advantage of reducing CDK6 protein levels where CDK6 protein levels increase upon treatment with palbociclib.

### Example 3—PROTACs for Selective Degradation of CDK6

### A. Materials and Methods

Cell lines, Ph+ primary ALL samples, and cell cultures. The SUP-B15 cell line (Ph+ ALL) was purchased from ATCC; the BV173 cell line (Ph+ CML-lymphoid blast crisis) (Pegoraro et. al JNCI, 1983) was kindly provided by Dr. N. Donato (NIH, Bethesda, Md.). Cell lines were cultured in Iscove's Modified Dulbecco's Medium (Corning, 10-016-CV) supplemented with 10% heat-inactivated fetal bovine serum (FBS) (Biowest USA), 100 U/mL penicillin-streptomycin (Thermo Fisher Scientific, #15140122) and 2 mmol/L L-glutamine (Thermo Fisher Scientific #25030081) at 37° C., 5% CO₂. Cell lines were tested for mycoplasma every 3 months as described (De Dominici et al., 2018).

Primary adult human Ph+ ALL cells were kindly provided by Dr. Luke F. Peterson (University of Michigan), and obtained from the Division of Hematological Malignancies of Thomas Jefferson University. The TKI-resistant Ph+ ALL sample (#557) was previously characterized as carrying the T315I ABL1 kinase domain mutation (Minieri et al., 2018). Primary Ph+ ALL cells were cultured in StemSpan SFEM (Stem Cell Technology #09650) supplemented with SCF (40 ng/mL), Flt3L (30 ng/mL), IL3 (10 ng/mL), IL-6 (10 ng/mL), and IL-7 (10 ng/mL; PeproTech). G-CSF-mobilized peripheral blood CD34+ primary cells from healthy donors were obtained from the Bone Marrow Transplantation Unit, Thomas Jefferson University and were cultured in StemSpan SFEM (Stem Cell Technology #09650) supplemented with StemSpan CC100 (Stem Cell Technologies ##02690).

Apoptosis and cell cycle analysis. Apoptosis was measured by Annexin V staining: 100,000 cells were resuspended in 50 μL of Annexin V Binding Buffer (10 mM HEPES, 140 mM NaCl, and 2.5 mM CaCl₂, pH 7.4) containing 1.5 μL of Cy-5.5-Annexin V (BD Bioscience #559933) for 15 min at room temperature (RT) and subsequently analyzed with the BD FACS Celesta flow cytometer.

For cell cycle analysis cells were incubated for 5 minutes with Sodium citrate 0.1%, Triton 0.1%, Propidium iodide 50 μg/mL and subsequently analyzed at the BD FACS Celesta flow cytometer.

Lentiviral production and cell transduction. For the constitutive silencing of CDK4/6, pLKO.1 lentiviral vectors conferring puromycin-resistance and expressing shRNAs against CDK6 (TRCN0000010082 [82], TRCN0000000486 [86], TRCN0000000488 [88], TRCN0000010473 [73]) or against CDK4 (TRCN0000000363) were obtained from The RNA Consortium, GE Dharmacon. The scramble shRNA pLKO.1 vector was obtained by Addgene (Addgene plasmid #1864). For lentiviral production, 293T cells were transiently transfected by the calcium phosphate-method with the pLKO.1 plasmid and the 2nd generation lentiviral packaging plasmids pMD2.G (Addgene plasmid 12259) and psPAX2 (Addgene plasmid #12260). After 24 hours, infectious supernatant was collected and used to transduce Ph+ ALL cells by two cycles of spinoculation (1000 g, 45 minutes, 37° C.) with subsequent incubation at 37° C. for 24 hours. Then, cells were selected with 3 μM puromycin for 72 hours, and dead cells were removed by centrifugation on a layer of Ficoll-Paque (GE Healthcare, 17544202). Cells were then cultured for 96 hours before assessing apoptosis and cell cycle by flow cytometry. For inducible CDK6 silencing, the CDK6-targeting shRNA-88 was cloned in the AgeI-EcoRI sites of the Tet-pLKO-puro vector (Addgene plasmid 21915). Lentiviral production was performed as described above. Lentiviral supernatant was concentrated by ultracentrifugation and used to transduce Ph+ ALL cell lines by three cycles of spinoculation. shRNAs were induced with 1 μg/mL doxycycline hydrochloride (RPI Corp. #D43020-100.0). The CDK6 cDNA was cloned in the pUltra-Chili lentiviral vector (Dr. Malcolm Moore; Addgene plasmid #48687) as described (De Dominici et a., 2018). To obtain a shRNA-resistant CDK6 cDNA, the target site of shRNA-88 was mutagenized by PCR-amplification with primers introducing multiple synonymous point mutations (codons 88-94: acCgaTCgGgaGacAaaGTtG, capital letters correspond to mutation introduced). The linear PCR-product was self-ligated, transformed into E. coli and sequenced. The plasmid was transduced in Ph+ ALL cells as described above.

RNA-sequencing. BV173 cells were plated at 5×10⁵ cells/mL and treated with Palbociclib 1 μM or DOX (1 μg/ml) for 48 hours. RNA was isolated with the RNeasy Plus Mini Kit (#74134, Qiagen) following the manufacturer's instructions. 100 ng of total RNA was used to prepare libraries using TruSeq Stranded Total RNA kit (Illumina, CA, USA) following the manufacturer's protocol. Libraries were sequenced on a NextSeq 500 instrument using 75-bp paired-end chemistry. Raw FASTQ sequencing reads were mapped against the reference human genome Ensembl Version GRCh38 utilizing further information from the gene transfer format (.gtf) annotation from GENCODE version GRCH28 using RSEM. Total read counts and normalized Transcripts Per Million (TPM) were obtained using RSEM's calculate-expression function. Before determining differential expression levels, batch effects and sample heterogeneity were tested using iSeqQC (github.com/gkumar09/iSeqQC). Differential gene expression was tested using the DESeq2 package in R/Bioconductor. Genes were considered differentially expressed (DE) if they had adjusted p≤0.05 and absolute fold change ≥2. All the plots were generated using R/Bioconductor, MA, USA.

Gene Set Enrichment Analysis (GSEA) was performed to evaluate Gene Ontology Biological Process (GOBP) terms in the resulting differential expression lists. The DESeq2 test statistic was used as a ranking metric to perform GSEA in pre-ranked mode, with genes having zero base mean or “NA” test statistic values filtered out to avoid providing numerous duplicate values to GSEA. GSEA pre-ranked analysis was performed using the “weighted” enrichment statistics. Cytoscape analysis was performed on the selected genes to examine their network patterns using Reactome functional interaction network.

Protein analysis. Cells were counted and lysed at a density of 10,000/μL in Laemmli Buffer supplemented with 5% β-Mercaptoethanol. Lysates were resolved on a 4-20% gradient polyacrylamide gels (Biorad, #4561095) and transferred onto a nitrocellulose membrane (Santa Cruz Biotechnology, #sc-3718) using a semi-dry trans-blot transfer cell (Bio-Rad). Membranes were then blocked in 5% non-fat dry milk/TBS-T and incubated with the following primary antibodies: CDK6 (rabbit, CST #13331), CDK6 (mouse, CST #3136), CDK4 (rabbit, CST #12790), CDK4 (rabbit, Bethyl Laboratories #A304-224), FOXM1 (rabbit, Santa Cruz Biotechnology #sc-502), phospho-RB Ser-780 (rabbit, CST #9307), phospho-RB Ser-897-811 (rabbit, CST #9308), β-ACTIN, (mouse, CST #3700).

Membranes were incubated with 1:10,000 HRP-conjugated secondary antibodies (Thermo Fisher Scientific, anti-mouse-HRP #31430, or anti rabbit-HRP #31460) and signals were visualized by chemiluminescent reaction using SuperSignal West Pico (Thermo Fisher Scientific #34580) or Dura (Thermo Fisher Scientific #34075) Chemiluminescent Substrates. When different antibodies were used to probe the same nitrocellulose membrane, previous signals were removed by incubation with 0.5% sodium azide for 10 minutes at RT or by stripping in 62 mM Tris-HCL pH 6.8, 2% SDS, 0.7% β-mercaptoethanol for 20 min at 50° C. The degradation constant (DC₅₀) for the tested PROTACs was calculated in GraphPad Prism 6.0 software by plotting the densitometric values of CDK4/6 intensity normalized by the intensity of the loading control.

Proteomic Analysis. 15×10⁶ BV173 cells were treated for 4 hours with 1 μM of PROTAC YX-2-107 or DMSO in triplicate. Cells were centrifuged, washed in ice-cold PBS and lysed in 300 μL of lysis buffer (50 mM Tris, pH 7.5, 1% SDS, 150 mM NaCl, 1 mM EDTA) supplemented with protease inhibitor cocktail (VWR #M221). Lysates were sonicated with Bioruptor (Diagenode) and cleared by centrifugation (10,000 g, 30 min, 4° C.). Protein concentration was assessed by the BCA method (Pierce #23227). Twenty-five μg of proteins were loaded on 10% acrylamide gels (Bio Rad, #4561035), electrophoresed into the gel for 0.5 cm and stained with Coomassie brilliant blue R-250 (Bio Rad #1610400) 0.1% in 40% ethanol, 10% Acetic Acid glacial, 50% water. The entire protein-containing gel regions were excised, digested with trypsin and analyzed by liquid chromatography tandem mass spectrometry (LC-MS/MS) using a 240-min gradient as described (Chae et al., 2016).

Peptide sequences were identified using MaxQuant 1.6.3.3 (Cox and Mann, 2008). MS/MS spectra were searched against a UniProt human protein database (Oct. 1, 2018) using full tryptic specificity with up to two missed cleavages, static carboxamidomethylation of Cys, and variable oxidation of Met and protein N-terminal acetylation. Proteins were quantified by label-free quantitation (LFQ). The “match between runs” feature was used to help transfer identifications across experiments to minimize missing values. Peptide and protein identifications were filtered at <1% false discovery rate (FDR) against a reversed-sequence database. Missing LFQ protein values were imputed with the dataset minimum value divided by two. The protein list was filtered to remove low confidence identifications by requiring proteins to be identified by at least 2 unique peptides in all triplicate of either sample. A total of 3,682 protein groups were quantified using these criteria. Protein levels were considered significantly different between the two samples if the absolute fold-change is >2 and the Student's t-test p-value is <0.05.

Animals. Mice experiments were performed according the guidelines of Thomas Jefferson University Institutional Animal Care and Use Committee (IACUC, protocol number 00012). For leukemogenesis assays, 2×10⁶ leukemia cells (shCDK6-transduced BV173 cells or primary cells from Ph+ ALL patients) were injected intravenously into 7- to 9-week-old NOD/SCID/IL-2Rγ^{null} or NRG-SGM3 mice (The Jackson Laboratory, stock #005557 and #024099, respectively). To induce CDK6 down-regulation in vivo, mice were continuously treated with doxycycline (2 g/L) in D(+)-sucrose-supplemented (30 g/L) drinking water starting 7 days post-cell injection. Palbociclib Isethionate was purchased from LC Laboratories (#P-7766) and was mixed in the chow by Research Diets Inc at 800 mg/kg. The dose was based on the average daily food intake of NSG mice in order to deliver 150 mg/kg per day of Palbociclib. Palbociclib chow was given ad libitum and replaced every 7 days for the duration of the experiment.

The percentage of leukemia cells in the peripheral blood or bone marrow was assessed by detection of the human CD19 (by antibody #555415 from BD Bioscience) or CD10 antigen (by antibody #555375 from BD Bioscience) using the BD FACS Celesta flow cytometer.

Metabolic stability of PROTACs in mouse liver microsomes. Test and control compounds were incubated at 0.5 μM with 0.5 mg/mL of liver microsomes and an NADPH-regenerating system (cofactor solution) in potassium-phosphate buffer (pH 7.4). At 0, 5, 15, 30, and 45 minutes, aliquots were taken, and reactions quenched with a solution of acetonitrile containing an internal standard. As controls, samples lacking the cofactor solution were also examined. At the end of the experiment, samples were analyzed by liquid chromatography with mass spectrometry (LC-MS/MS). The intrinsic clearance (CLint) was determined from the first-order elimination constant by nonlinear regression. This analysis was performed by Alliance Pharma (Malvern, Pa.).

Pharmacokinetic analysis of PROTAC YX-2-107 in CD-1 mice. A 1-arm PK study was performed in 18 CD-1 mice (n=3 mice per time point). Animals were injected intraperitoneally (IP) with a single dose (10 mg/kg) of PROTAC YX-2-107 dissolved in a solution of 10% DMSO, 10% Solutol, 80% PBS. Plasma samples were collected at 0.25, 0.5, 1, 2, 4, and 6 hours post-IP injection and analyzed by LC-MS/MS. Concentrations of PROTAC YX-2-107 were calculated by linear regression analysis. This analysis was performed by Alliance Pharma (Malvern, Pa.).

CDK6 DC50 determination in PROTAC YX-2-107-treated BV173 cells. PROTAC YX-2-107 concentration for half-maximal degradation (DC50) of CDK6 was assessed by immunoblot analysis of CDK6 in cells treated with PROTAC at various doses. Levels of CDK6 were measured by densitometric analysis using ImageJ software and normalized by the levels of β-ACTIN. The DC₅₀ was determined by analyzing the dose-effect curve in Graphpad PRISM

Quantitative PCR analysis. RNA was isolated from untreated, Palbociclib-treated (500 ng/ml; 48 hrs), or doxycycline-treated (2.5 μg/ml; 48 hrs) shCDK6-88 BV173 cells, using the RNeasy Plus Mini Kit (Qiagen, Limburg, The Netherlands) and then reverse-transcribed (2 μg) using the High-Capacity cDNA Reverse Transcription Kit (ThermoFisher Scientific, Waltham, Mass., USA). Quantitative PCR was performed with the QuantStudio 12k Flex (Life Technologies) instrument and QuantStudio 12K Flex software, using the following primers:

HDCA1 FW,
(SEQ ID NO: 1)
5′-CATGCTGTGAATTGGGCTG-3′;
RV,
(SEQ ID NO: 2)
5′-CCCTCTGGTGATACTTTAGCAGT-3′;
SMARCD2 FW,
(SEQ ID NO: 3)
5′-GCATGCTGCCCGGACC-3′;
RV,
(SEQ ID NO: 4)
5′-ACATGCCAGGTCGCTGGT-3′;
JAK1 FW,
(SEQ ID NO: 5)
5′-TCCGCGACGTGGAGAATATC-3′,
RV,
(SEQ ID NO: 6)
5′-TGGTGTGGTAAGGACATCGC-3′;
HADHA FW,
(SEQ ID NO: 7)
5′-TCAACATGTTAGCCGCTTGC-3′;
RV,
(SEQ ID NO: 8)
5′-ATGGCAACCTCAAGTCCTCC-3′;
ACSL1 FW,
(SEQ ID NO: 9)
5′-GGAACTACAGGCAACCCCAA-3′;
RV,
(SEQ ID NO: 10)
5′-TCATCTGGGCAAGGATTGACT-3′;
GOT2 FW,
(SEQ ID NO: 11)
5′-TTGAAGAGTGGCCGGTTTGT-3′;
RV,
(SEQ ID NO: 12)
5′-TGCAGAAAACTGGCTCCGAT-3′;
NFATC2 FW,
(SEQ ID NO: 13)
5′-AGACGAGCTTGACTTCTCCA-3′;
RV,
(SEQ ID NO: 14)
5′-TGCATTCGGCTCTTCTTCGT-3′;
HACD1 FW,
(SEQ ID NO: 15)
5′-TGCCTTGCTTGAGATAGTTCAC-3′;
RV,
(SEQ ID NO: 16)
5′-TCACTTGGACCCCAGTCACA-3′.

Statistical analyses. Data, expressed as mean±s.d. of three experiments, were analyzed for statistical significance by unpaired, two-tailed Student's t-test. P<0.05 was considered statistically significant. Kaplan-Meier plots for mice survival experiments were generated using the GraphPad Prism 6.0 software. Differences in survival were assessed by log-rank test. mRNA levels correlations were analyzed by the Pearson test, and significance was calculated by the Student t distribution.

### C. Synthesis and Characterization

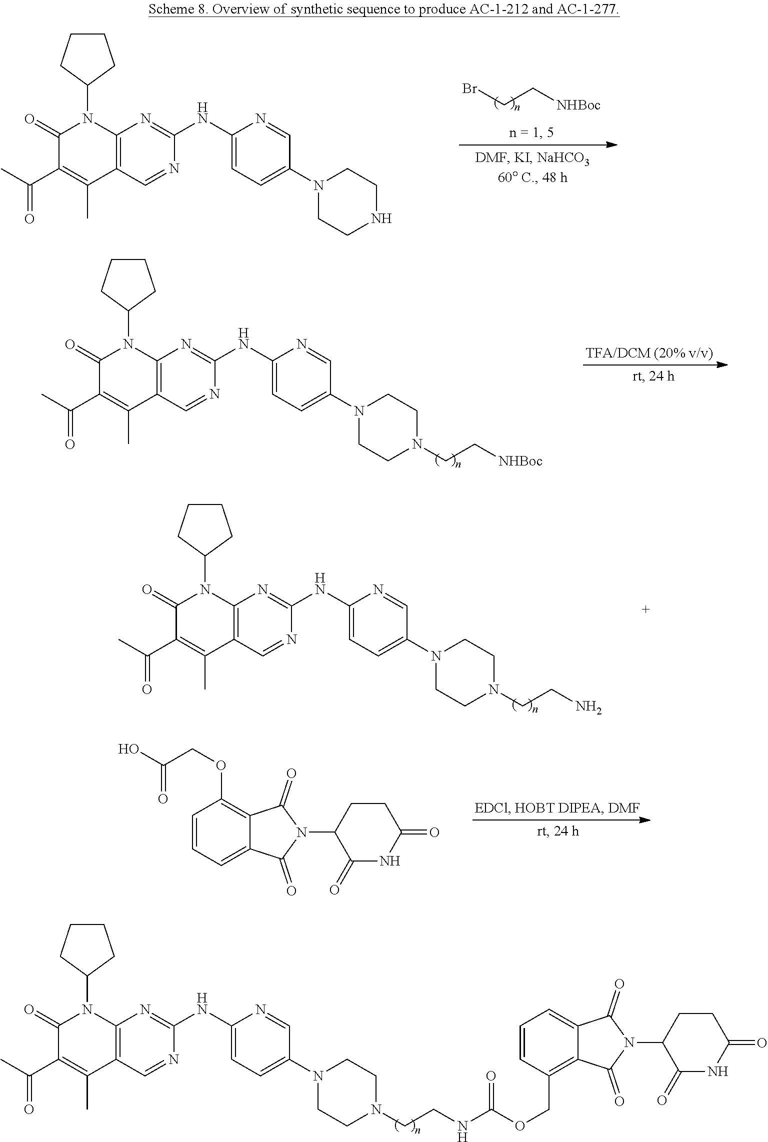

General Synthesis of AC-1-212 and AC-1-277: A 50 mL round bottom flask was charged with the Palbociclib-linker (1 equiv.), Cereblon-ligand acid (1 equiv.), EDCI (2.0 equiv.), HOBT (2.0 equiv.), DIPEA (4 equiv.) and dissolved in DMF to make a 12 mmol solution. The reaction was stirred overnight at rt, then diluted with 15 mL of water. The phases were separated, and the aqueous layer extracted 3× with DCM. The organic fractions were combined, then washed 3× with water (20 mL), dried with Na₂SO₄, and concentrated. The compound was isolated by MPLC normal phase on 12 g silica cartridges with 0-35% DCM/MeOH gradient elution.

AC-1-212: N-(2-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)ethyl)-2-((2-(2,6-dioxopiperidin-3-yl)-1,3-dioxoisoindolin-4-yl)oxy)acetamide was prepared with Palbociclib-linker X1 (93 mg, 0.20 mmol), Cereblon-ligand acid X (69 mg, 0.20 mmol), EDCI (73 mg, 0.40 mmol), HOBT (58 mg, 0.40 mmol) and DIPEA (0.13 mL, 0.80 mmol), following the General Synthesis to afford 60 mg (39%) of AC-1-212 as a yellow solid: ¹H NMR (400 MHz, CDCl₃) δ 8.81 (d, J=7.2 Hz, 1H), 8.16 (t, 8.3 Hz, 1H), 8.08 (s, 1H), 7.77-7.64 (m, 1H), 7.53 (q, J=3.9 Hz, 1H), 7.37-7.30 (m, 1H), 7.21 (d, J=4.2 Hz, 1H), 7.13 (d, J=4.2 Hz, 1H), 5.86 (quin, J=8.1 Hz, 1H), 5.04-4.95 (m, 1H), 4.92 (s, 1H), 4.81-4.73 (m, 1H), 4.67 (s, 1H), 3.82-3.75 (m, 1H), 3.59-3.47 (m, 1H), 3.31-3.19 (m, 3H), 3.20-3.10 (m, 1H), 2.94-2.57 (m, 9H), 2.54 (s, 3H), 2.34 (s, 3H), 2.19-2.10 (m, 1H), 2.10-1.95 (m, 3H), 1.92-1.79 (m, 2H), 1.71-1.57 (m, 2H). LCMS m/z [M+H⁺] Calcd. for C₄₁H₄₄N₁₀O₈ 804, found 805.

AC-1-277: N-(6-(4-(6-((6-acetyl-8-cyclopentyl-5-methyl-7-oxo-7,8-dihydropyrido[2,3-d]pyrimidin-2-yl)amino)pyridin-3-yl)piperazin-1-yl)hexyl)-2-((2-(2,6-dioxopiperidin-3-yl)-1,3-dioxoisoindolin-4-yl)oxy)acetamide was prepared with Palbociclib-linker X2 (65 mg, 0.12 mmol), Cereblon-ligand acid X (43 mg, 0.12 mmol), EDCI (45 mg, 2.4 mmol), HOBT (36 mg, 0.24 mmol) and DIPEA (0.08 mL, 0.47 mmol), following the General Synthesis to afford 46 mg (45%) of AC-1-277 as a yellow solid: ¹H NMR (400 MHz, CDCl₃) δ 8.99 (s, 1H), 8.07-8.00 (m, 1H), 7.99-7.93 (m, 1H), 7.92-7.87 (m, 1H), 7.74 (q, J=7.3 Hz, 1H), 7.61 (d, J=9.5 Hz, 1H), 7.46 (d, J=7.3 Hz, 1H), 7.35 (d, J=7.3 Hz, 1H), 5.90 (t, J=9.8 Hz, 2H), 5.08-5.01 (m, 2H), 3.38 (quin, J=1.7 Hz, 1H), 3.30-3.24 (m, 2H), 3.15-3.09 (m, 4H), 3.03 (quin, J=1.7 Hz, 1H), 2.85-2.55 (m, 5H), 2.41 (s, 3H), 2.33 (s, 3H), 2.27-2.15 (m, 3H), 2.11-2.02 (m, 2H), 2.02-1.93 (m, 2H), 1.87-1.75 (m, 2H), 1.74-1.66 (m, 2H), 1.63-1.57 (m, 2H), 1.57-1.48 (m, 3H), 1.40-1.34 (m, 4H). LCMS m/z [M+H⁺] Calcd. for C₄₅H₅₂N₁₀O₈ 861, found 862.

### C. Results

CDK6 silencing is more effective than CDK4/6 enzymatic inhibition in suppressing Ph+ ALL in immunodeficient mice. Proliferation, CDK4/6-dependent RB phosphorylation and FOXM1 (Sherr et al., 2016; Anders et al., 2011) are markedly reduced in CDK6-silenced Ph+ ALL cell lines while CDK4 silencing had no such effects (De Dominici et al., 2018). However, it is unknown whether CDK6 silencing suppresses Ph+ ALL in mice and whether the effects are comparable or superior to CDK4/6 enzymatic inhibition.

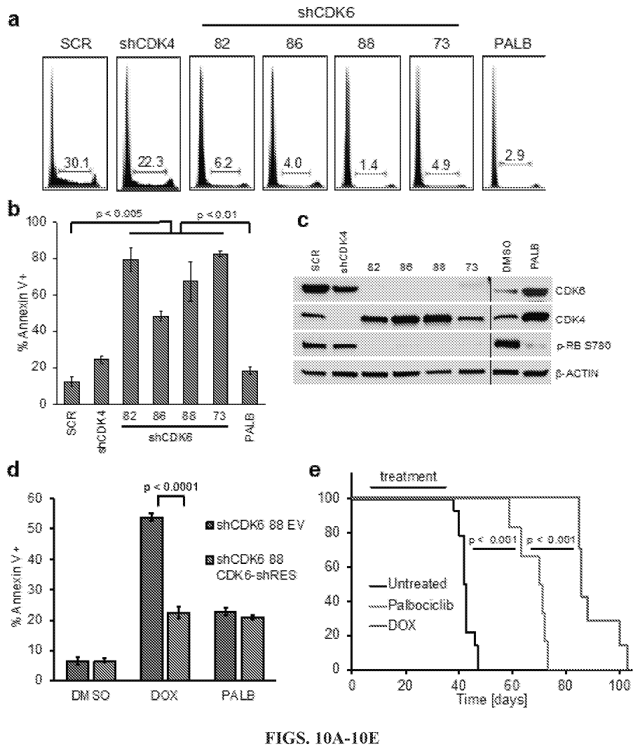

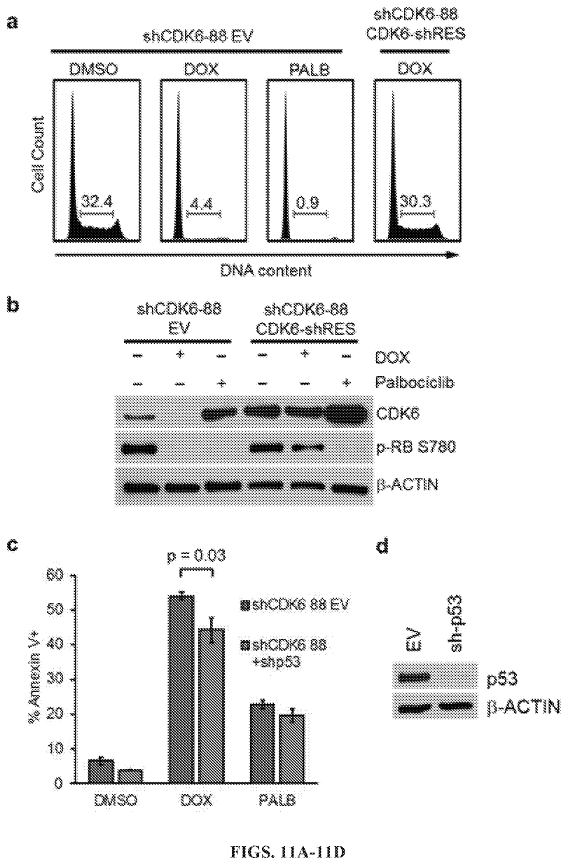

The CDK6 silencing in Ph+BV173 cells was determined to induce a marked increase in the frequency of apoptotic cells which was not detected after treatment with the CDK4/6 inhibitor Palbociclib or after CDK4 silencing (FIGS. 10A-10C). To further assess if apoptosis induced by CDK6 silencing was specific, shCDK6-88 was cloned in the tetracycline-regulated Tet-pLKO-puro vector and transduced into BV173 cells which were subsequently transduced with either a vector expressing a CDK6 cDNA engineered to prevent the binding of the shRNA, or with the empty vector (EV). As shown in FIG. 10D, expression of the shRNA-resistant form of CDK6 rescued the apoptosis induced by CDK6 knockdown in BV173 cells, whereas treatment with Palbociclib had modest but similar effects in both cell lines. In these cells, expression of the shRNA-resistant CDK6 also rescued the DOX-induced decrease in CDK6 and phospho-RB levels, as well as in the number of S phase cells (FIGS. 11A & 11B). Together, these data suggest that kinase-independent effects may be involved in the apoptosis induced by CDK6 silencing.

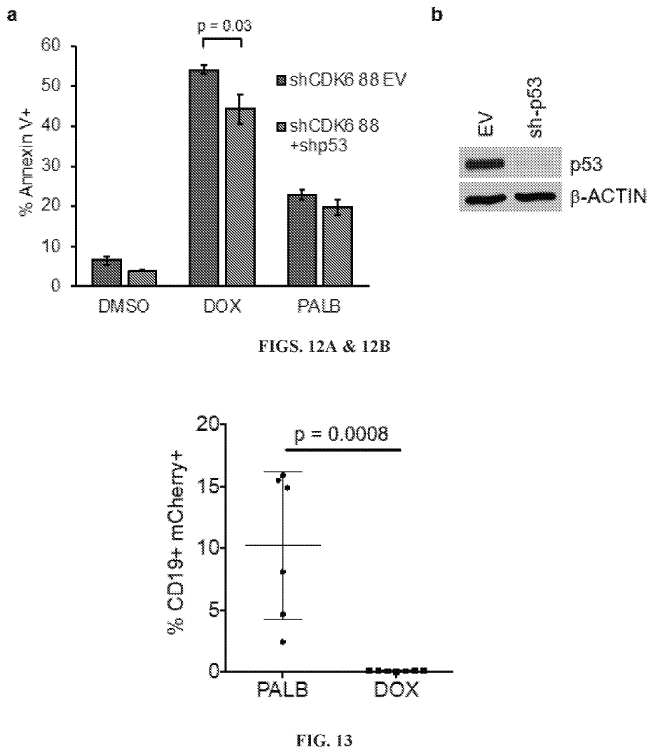

Since CDK6 is reported to regulate the activity of p53 by enhancing the transcription of p53 antagonists (Bellutti et al., 2018), it was assessed that p53 had any role in the apoptosis induced by CDK6 silencing. Down-regulation of p53 expression rescued only modestly, albeit significantly, the apoptosis induced by CDK6 silencing (FIG. 12A), indicating that in Ph+ ALL cells apoptosis induced by CDK6 silencing is predominantly p53-independent.

To assess whether enhanced apoptosis induced by CDK6 silencing ex vivo might correlate with reduced growth of Ph+BV173 cells in vivo, NSG mice were injected with DOX-inducible shCDK6-BV173 cells and left untreated or treated with DOX in the drinking water starting 7-day post cell injection. An additional group of mice was treated with Palbociclib given in the chow to compare directly the effects of CDK6 silencing vs. CDK6 enzymatic inhibition. Treatments were terminated after 4 weeks and two weeks later the peripheral blood was analyzed by flow cytometry to assess the percentage of CD19+ leukemic cells. Such cells were undetectable or barely detected in DOX-treated mice while they comprised approximately 10% (range: 2.4-16%) of total white blood cells in Palbociclib-treated animals (FIG. 13), indicating that leukemia load was markedly suppressed by CDK6 silencing. These data on leukemia burden correlated with markedly different survival of the DOX-treated versus the Palbociclib-treated mice.

Compared to untreated mice (median survival=42.5 days), Palbociclib treated mice exhibited a significant prolongation in survival (median survival=70.5 days, p<0.001). However, treatment with DOX to silence CDK6 expression was even more effective resulting in a survival longer than that induced by Palbociclib treatment (median survival=86 days, p=0.0002; FIG. 10E). Thus, selective silencing of CDK6 provides an advantage over non-selective CDK4/6 enzymatic inhibitors like Palbociclib.

The growth suppression induced by CDK6 silencing correlates with a specific gene expression signature. The longer survival of DOX-treated compared to Palbociclib-treated NSG mice injected with shCDK6-BV173 cells suggests that kinase-independent effects were involved in the more pronounced leukemia suppression induced by CDK6 silencing. To search for kinase-independent pathways potentially explaining these effects, the gene expression profile of Palbociclib-treated and CDK6-silenced (DOX-treated; 48 h) BV173 cells were compared. It should be noted that that the selective silencing of CDK4 has no effect on the proliferation and phospho-RB levels of Ph+ ALL cell lines (De Dominici et a., 2018), indicating that inhibition of CDK4 enzymatic activity does not contribute to the effects of Palbociclib.

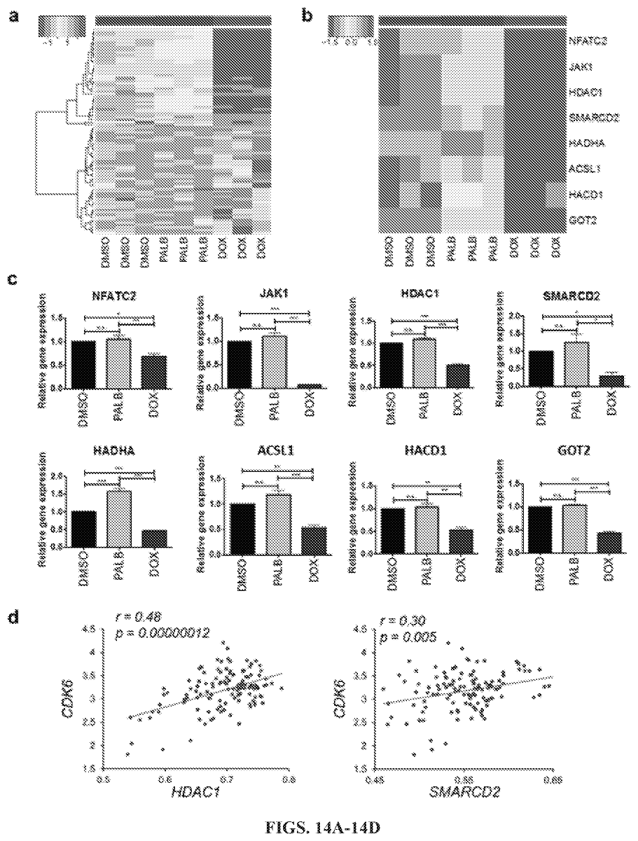

As expected, most genes were similarly regulated by CDK6 enzymatic inhibition and silencing; however, 80 genes showed at least a 1.5-fold change in expression in CDK6-silenced cells compared to Palbociclib-treated cells (FIG. 14A). Among the genes selectively modulated by CDK6 silencing, several of those that were downregulated (FIGS. 14B & 14C) might be functionally linked to the apoptosis susceptibility of CDK6-silenced BV173 cells. In particular, decreased expression of JAK1 and NFATC2 could affect pro-survival signaling, while lower levels of SMARCD2 and HDAC1 could influence chromatin remodeling possibly affecting transcriptional regulation of pro-survival genes.

CDK6 silencing may also affect fatty acid metabolism as suggested by decreased expression of the HACD1, ACSL1, and HADHA genes. These genes encode for enzymes involved in long-chain fatty acid elongation (HACD1), fatty acid activation through synthesis of fatty acid acyl-CoA esters (ACSL1), and mitochondrial fatty acid beta-oxidation (HADHA).

Lastly, CDK6 silencing may also affect oxidative phosphorylation based on decreased expression of the GOT2 gene which encodes for mitochondrial aspartate aminotransferase, a component of the malate-aspartate shuttle which is used for NADH transfer from the cytosol into the mitochondria.

To further investigate whether the “CDK6-silencing signature” might be clinically significant, mRNA levels of CDK6 and its putative targets were analyzed in a dataset of 122 Ph+ ALL samples; a strikingly positive correlation between CDK6 and HDAC1 expression (P=0.00000012) were observed and a significant correlation between CDK6 and SMARCD2 expression (P=0.005) (FIG. 14D). No significant correlation was found in the expression of CDK6 and the remaining putative targets.

Collectively, these changes in gene expression, in particular these potentially impairing chromatin remodeling may explain the propensity to undergo apoptosis of CDK6-silenced Ph+ ALL cells.

Development of a potent CDK4/6-targeted PROTAC that selectively degrades CDK6. Based on the ex vivo and in vivo data shown in FIG. 10, a compound selectively degrading CDK6 would be expected to be more effective and have fewer side effects than a CDK4/6 enzymatic inhibitor in exploiting the CDK6 dependence of Ph+ ALL.

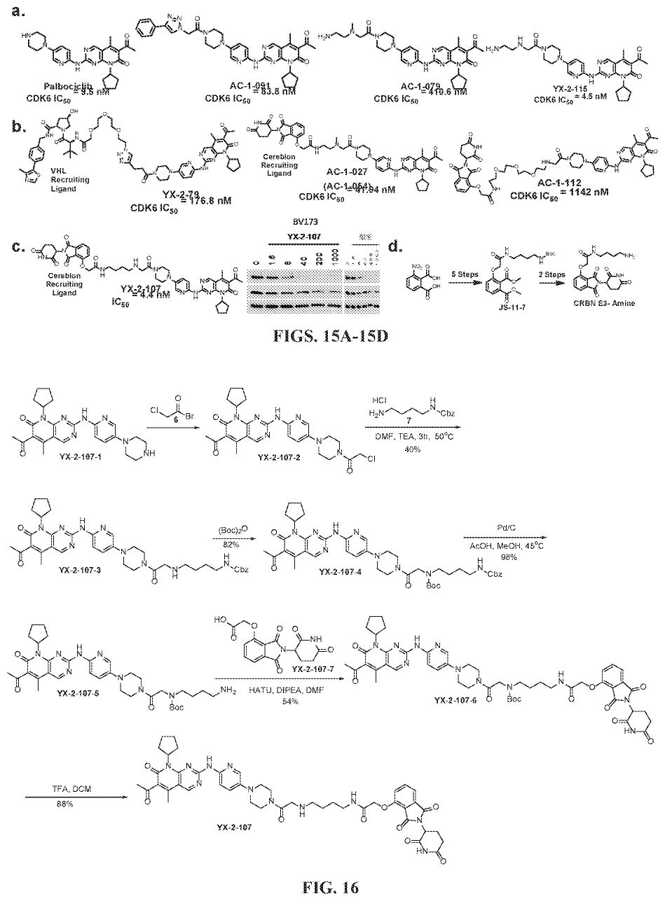

Without wishing to be bound by any theory, it is believed that a potent CDK4/6 kinase inhibitor tethered to an E3 ligase-recruiting molecule might bind to CDK6 and degrade this protein, potentially providing more specific and durable inhibition than currently possible with small molecule inhibitors. Therefore, CDK4/6-targeted-PROTACs were designed guided by the X-ray crystal structure of Palbociclib in complex with CDK6 (PDB id: 2EUF and 5L2T) (Lu et al., 2006; Chen et al., 2016). The piperazine tail of Palbociclib protrudes from the active site (ATP pocket) towards the solvent, suggesting that it might tolerate linker attachment to various E3 ligase-recruiting molecules (Lu et al., 2006). First, several Palbociclib derivatives were synthesized with a variety of linkers; a small representative set is shown in FIG. 15A. It was found that the kinase inhibitory activity of these conjugates varied dramatically, which was not predicted based on the seemingly large volume of solvent-exposed space suggested by the crystal structure in the region where the piperazine tail conjugated with a linker (FIG. 15A) would exit into the solvent (Lu et al., 2006). Notably, a single methyl group change in YX-2-115 (45) compared to AC-1-079 resulted in a 100-fold decrease in inhibition of kinase activity (FIG. 15A). Hence, each molecule had to be considered as a whole and optimized accordingly, rather than attempting to optimize individual components to guide the assembly of a final compound, since that approach would not necessarily result in an optimal PROTAC. FIG. 15B shows several potential PROTACs which have different linkers and either a VHL or Cereblon recruiting ligand (Winter et al., 2015; Buckley et al., 2012). These compounds represent derivatives lacking potent kinase inhibition when tested in vitro for the ability to inhibit cyclin D3/CDK6- or cyclin D1/CDK4-dependent RB phosphorylation. By contrast, the Cereblon-recruiting PROTAC YX-2-107 was identified as a potent inhibitor of in vitro CDK4 or CDK6 kinase inhibitor (IC₅₀=0.69 and 4.4 nM, respectively) (FIG. 15C) comparable to Palbociclib (IC₅₀=11 and 9.5 nM, respectively) (FIG. 15A) as well as a selective CDK6 degrader in Ph+BV173 ALL cells with a degradation constant, DC₅₀, of ˜4 nM (FIG. 15C), based on densitometry of CDK6 band intensity. FIG. 15D shows the synthesis of CRBN E3 amine that serves as recruiter for Cereblon E3 ligase. Schematic steps for the synthesis of PROTAC YX-2-107 are shown in FIG. 16.

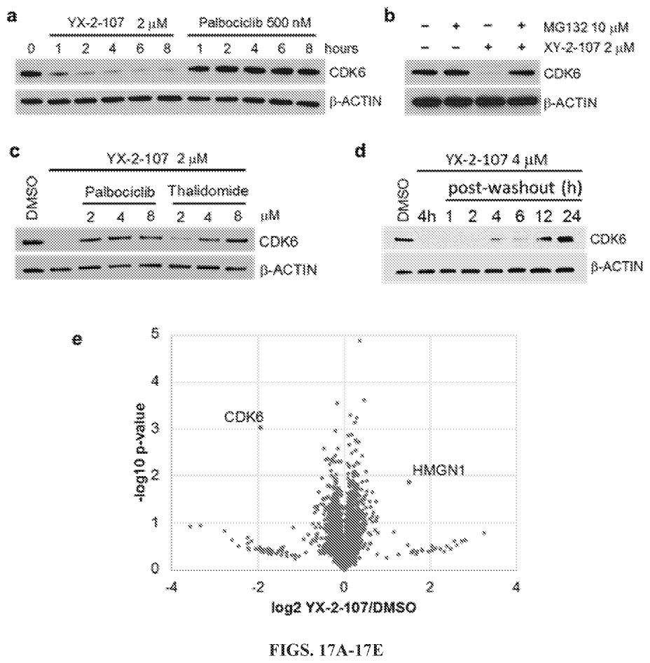

Downregulation of CDK6 expression in Ph+BV173 cells was detected as early as one hour post-treatment with YX-2-107, and at 4 hours CDK6 levels were markedly reduced (FIG. 17A). Treatment with the proteasome inhibitor MG132 restored CDK6 expression to the levels of untreated cells (FIG. 17B), suggesting that, in YX-2-107-treated Ph+ ALL cells, CDK6 is targeted for proteasome degradation via Cereblon-dependent ubiquitination. Co-treatment with Palbociclib or thalidomide to block binding of PROTAC YX-2-107 to CDK6 or Cereblon respectively, prevented the downregulation of CDK6, suggesting that the degradation of CDK6 induced by YX-2-107 requires the formation of a ternary complex consisting of CDK6+PROTAC+Cereblon (FIG. 17C). Expression of CDK6 in PROTAC YX-2-107-treated BV173 cells was very low for at least 6 hours, returning to the levels of untreated cells 12 hours after washing the PROTAC from the culture medium (FIG. 17D), consistent with a more durable inhibitory effect for this PROTAC than for the CDK4/6 enzymatic inhibitor Palbociclib.

Protein degradation induced by YX-2-107 was highly specific since proteomic analysis of BV173 cells treated with YX-2-107 for four hours revealed that of 3,682 proteins examined only CDK6 was significantly downregulated, based on the protein LFQ intensity compared to the control (DMSO-treated) cells (FIG. 17E). Expression of the High Mobility Group Nucleosome Binding Domain 1 protein, HMGN1, was significantly higher in YX-2-107-treated than in control BV173 cells (FIG. 17E), probably reflecting a secondary change in its protein level consequent to CDK6 degradation.

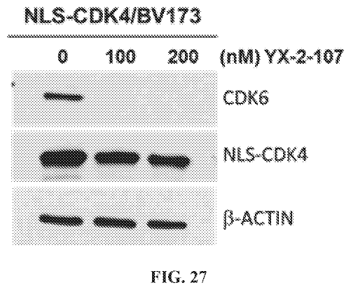

Since CDK4 is exclusively localized in the cytoplasm of Ph+ ALL cells whereas CDK6 is predominantly nuclear (De Dominici et al., 2018), it was asked whether this differential localization might explain the preferential CDK6 degradation by Cereblon, which was reported to be also localized in the nucleus (Wada et al., 2016). Thus, a BV173 derivative line expressing a nuclearly-localized CDK4 protein (NLS-CDK4-BV173) (De Dominici et al., 2018) was treated with PROTAC YX-2-107 for 4 hours and levels of NLS-CDK4 were then assessed by western blotting. As shown in FIG. 27, ectopically-expressed nuclear NLS-CDK4 was not degraded by PROTAC YX-2-107, strongly suggesting that the nuclear localization (endogenous or engineered) of either CDK4 or CDK6 does not dictate their targeting by PROTAC YX-2-107. Based upon these results, it was concluded that the predominantly nuclear localization of CDK6 as opposed to CDK4 in Ph+ ALL cells does not account for the preferential degradation of CDK6 over CDK4 by PROTAC YX-2-107.

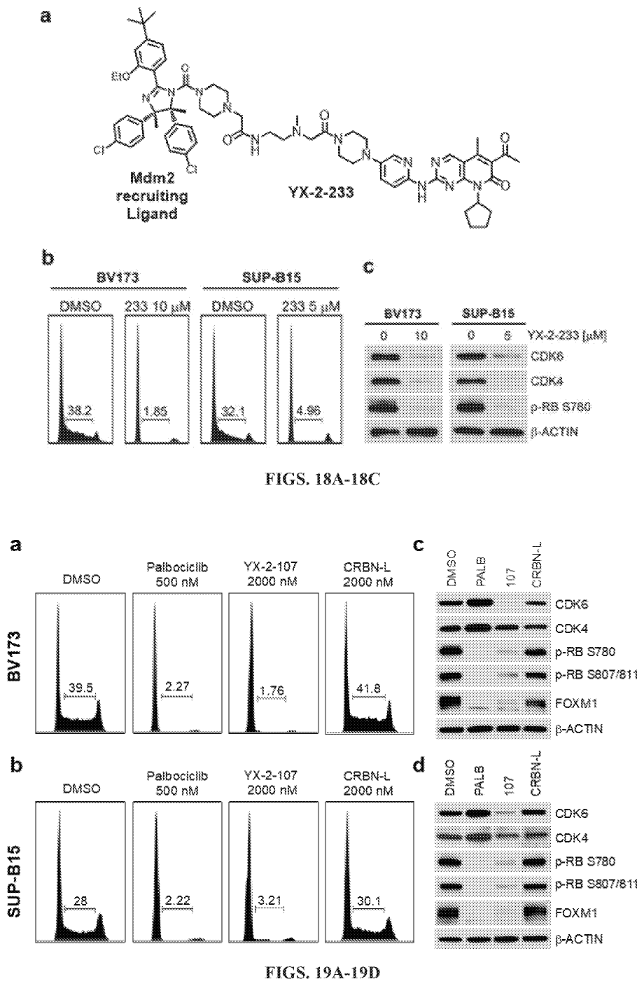

In addition to PROTACs containing VHL or Cereblon ligands, PROTAC YX-2-233 (FIG. 18) was synthesized which is a Palbociclib derivative conjugated to an MDM2-recruiting ligand derived from RG7112 (Tovar et al, 2013). This PROTAC potently suppressed S phase and RB phosphorylation in Ph+ ALL cells (FIGS. 18B & 18C); however, it degraded CDK4 as well as CDK6 (FIG. 18C), suggesting that the E3 ligase that is recruited by the PROTAC may influence the selective degradation of a targeted protein.

Effects of PROTAC YX-2-107 in Ph+ ALL cell lines and normal hematopoietic progenitors (HPCs). The molecular and biological effects of CDK6-degrading PROTACs were assessed, ex vivo, in Ph+ ALL cell lines. Treatment with YX-2-107 inhibited S phase entry, RB phosphorylation, and FOXM1 expression in Ph+BV173 and SUP-B15 cells (FIGS. 19A-19D). A finding was that Palbociclib treatment induced increased expression of CDK4 and, especially, CDK6; by contrast, CDK4 expression did not increase in YX-2-107-treated cells while levels of CDK6 were markedly decreased (FIGS. 19B & 19C). The control compound, CRBN E3-Amine (CRBN-L; FIG. 15D) that consists exclusively of the Cereblon E3 ligase recruiting ligand, had no effect (FIGS. 19A-19D).

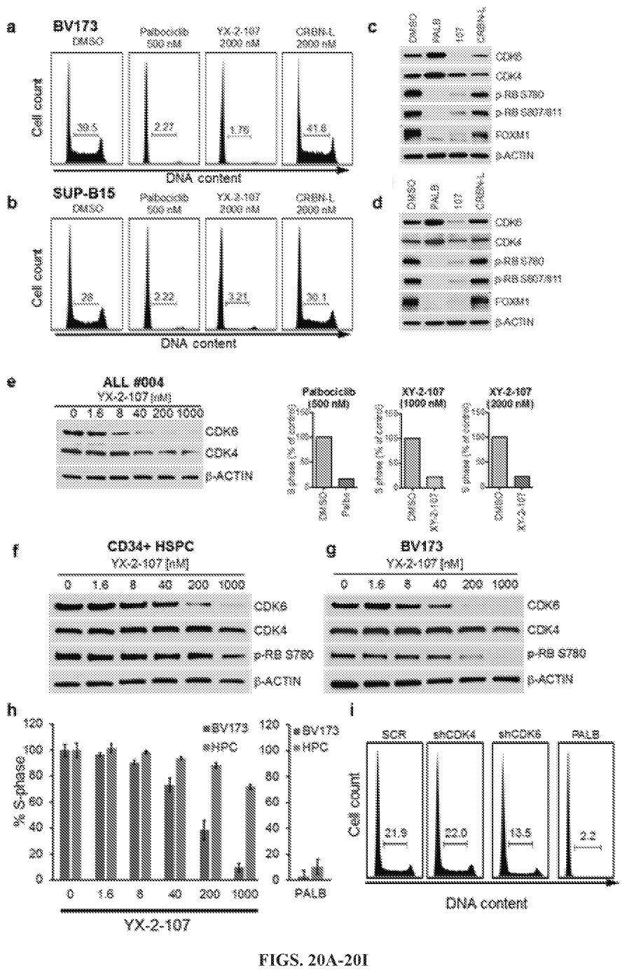

Selective degradation of CDK6 and inhibition of S phase was also observed in YX-2-107 treated blast cells from a patient with de novo Ph+ ALL (sample #004) (FIG. 20E). Since S phase cells in this primary Ph+ ALL sample were only 6.5% by flow cytometry analysis, the effect of PROTAC YX-2-107 was independently evaluated by EdU-pulse labeling. This assay revealed that EdU-positive cells were 4.8% in the untreated sample, 0.5% in cells treated with Palbociclib, and 1.6 and 0.7% in cells treated with 1 or 2 μM YX-2-107 respectively, confirming the results of the flow cytometry analysis.

In normal CD34+ hematopoietic stem and progenitor cells (HSPCs), a 24-hour treatment with YX-2-107 also suppressed CDK6 expression with no effect on CDK4 levels; however, this treatment did not inhibit S phase or reduce phospho-RB as effectively as in BV173 cells (FIG. 20F-20H). By contrast, pharmacological inhibition of CDK4/6 enzymatic activity by treatment with Palbociclib had similar growth-suppressive effects in BV173 and normal CD34+ HSPCs (FIG. 20H). These findings suggest that, unlike Ph+ ALL cells, normal CD34+ HSPCs rely for their growth on both CDK4 and CDK6. This hypothesis was confirmed by the observation that silencing CDK4 alone did not decrease the percentage of CD34+S phase cells whereas selective CDK6 silencing had only a partial effect compared to treatment with the dual CDK4/6 inhibitor Palbociclib which, instead, induced the complete suppression of CD34+S phase cells (FIG. 20I).

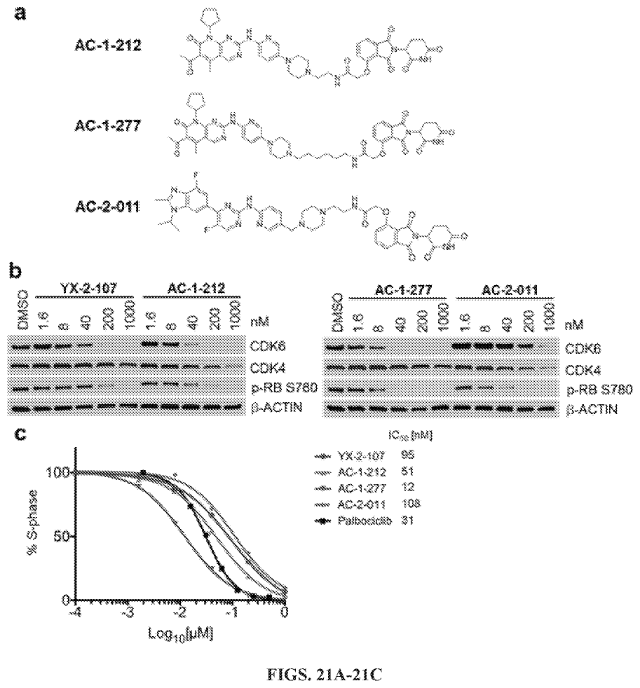

Additional PROTACs were evaluated to develop structure-activity relationships (SAR) during the optimization process. For example, PROTACs AC-2-011, AC-1-212, and AC-1-277 were tested in BV173 cells (FIG. 21). These three PROTACs all inhibited RB phosphorylation and markedly reduced the percentage of S phase cells, but AC-12-011 did not appear to function as a potent CDK6 degrader, since it degraded CDK6 and CDK4 only at high concentrations (FIG. 21A). By contrast, AC-1-212 and in particular AC-1-277 degraded CDK6 selectively and potently as well as suppressed the number of BV173 S phase cells with similar or higher potency as Palbociclib (FIG. 21B).

PROTAC YX-2-107 is bioavailable in mice and pharmacologically active in suppressing Ph+ ALL proliferation. In order to assess the potential use of CDK6-selective PROTACs as drugs in vivo, the metabolic stability of YX-2-107 in mouse liver microsomes was first evaluated and compared it to Palbociclib, and to 4-hydroxy-thalidomide (AC-1-158), using midazolam as a positive control (FIG. 22A). Compounds with greater than a 20-30 minute half-life are predicted to have reasonablly slow clearance and acceptable pharmacokinetic exposure in the plasma. YX-2-107 has good metabolic stability after incubation with mouse liver microsomes, displaying a half-life of 35 minutes, comparable to Palbociclib which exhibited a half-life of 56 minutes. The positive control compound midazolam (poor stability) had a half-life of only about 2 minutes. Other derivatives showed poor (i.e. AC-1-027 (FIG. 15B) with only 2 minute half-life) or moderate stability (i.e. AC-1-212 (FIG. 21) with a 10 minute half-life), emphasizing the need to optimize these compounds prior to in vivo evaluation. YX-2-107 was next evaluated in a mouse pharmacokinetic (PK) study at a 10 mg/kg IP dose (FIG. 22B). Plasma levels show a Cₘₐ=741 nM (150-fold greater than the CDK6 degradation IC₅₀), with clearance from the plasma after 4 hours. The plasma exposure is 30-fold higher than the CDK6 degradation IC₅₀ at 2 h (133 nM), and approximately 4-fold higher the CDK6 degradation IC₅₀ at 6 h (21 nM). Conceivably, CDK6 inhibition may persist for an extended time period beyond clearance of YX-2-107 based on the time needed for recovery of CDK6 de novo protein synthesis in PROTAC YX-2-107-treated BV173 cells (FIG. 17D), providing an advantage over Palbociclib or other ATP competitive kinase inhibitors. Although the PK profile of PROTAC YX-2-107 may not be optimal for a clinical compound, it is suitable for a feasibility study to evaluate its growth-suppressive effects in vivo.

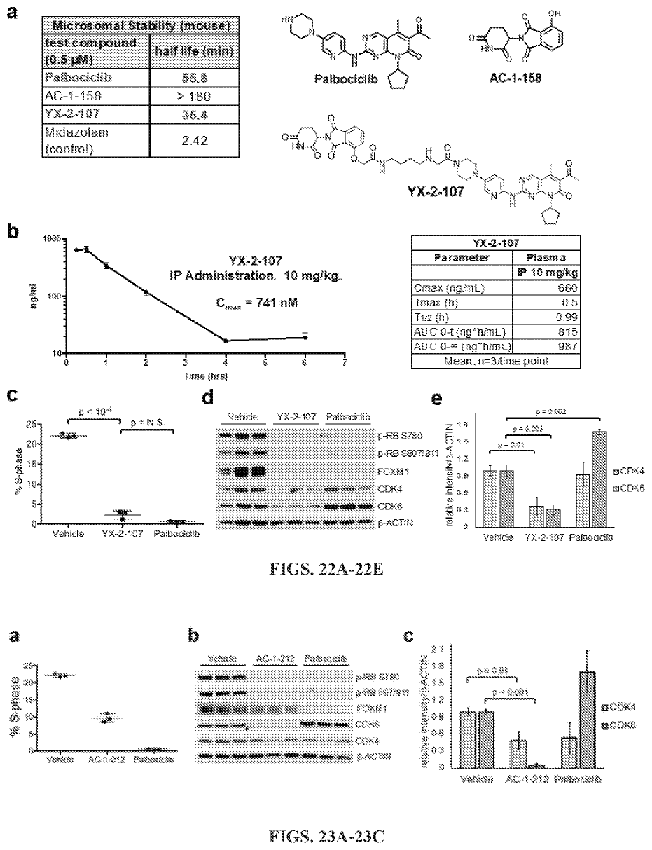

To assess whether PROTAC YX-2-107 is pharmacologically active in vivo its effects were tested after a short treatment in Ph+ ALL xenografts. For this experiment, mice (n=9; three/group) were injected with primary Ph+ ALL cells, monitored for the presence of leukemic cells (CD19+/CD10+ in the peripheral blood, and treated (three consecutive days) with Palbociclib, YX-2-107, or with vehicle only when these cells were >50%. Subsequently, bone marrow cells (>90% CD19+/CD10+) were purified and assessed for cell cycle activity, phospho-RB, FOXM1, and CDK4/CDK6 levels. Palbociclib and YX-2-107 were indistinguishable in terms of suppressing the percentage of S phase cells (FIG. 22C) and in decreasing the expression of phospho-RB and FOXM1 (FIG. 22D). However, treatment with PROTAC YX-2-107 reduced CDK6 and, to a lesser degree, CDK4 levels, while conversely CDK6 expression was upregulated by Palbociclib (FIG. 22E). A similar pilot study was performed with PROTAC AC-1-212. Treatment with this PROTAC suppressed the percentage of primary Ph+ ALL S phase cells, the expression of CDK4/6-regulated p-RB and, to a lesser degree, FOXM1, and induced the selective degradation of CDK6. However, AC-1-212 was less effective than YX-2-107 and the effects were not dose-dependent (FIG. 23), probably reflecting its suboptimal pharmacokinetic exposure (10 minute half-life) in mouse liver microsomes.

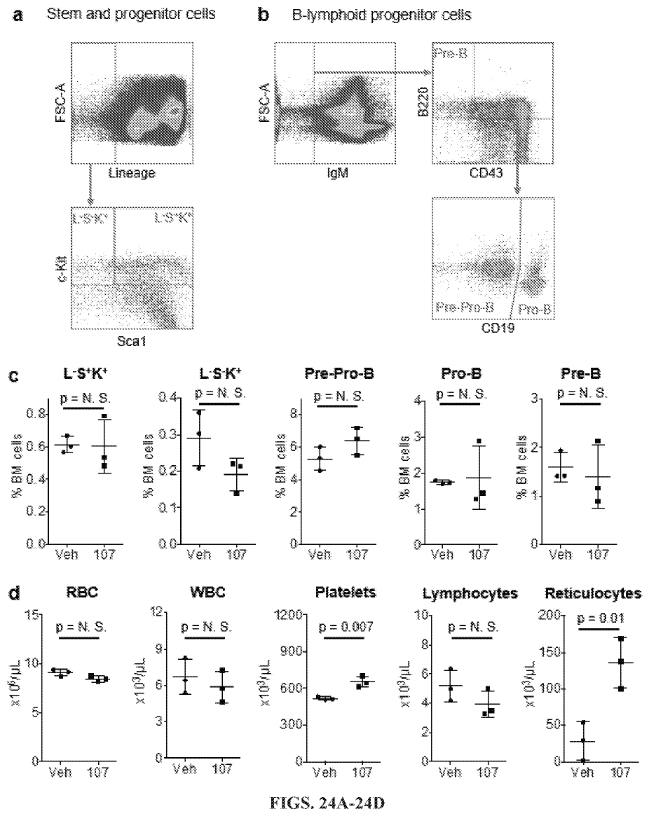

Effects of CDK6-degrader PROTAC YX-2-107 in patient-derived xenografts (PDXs) of Ph+ ALL. Based on these encouraging in vivo data with YX-2-107, further investigations of YX-2-107 and its effects in models of Ph+ leukemia were conducted. First, it was assessed whether in vivo treatment with YX-2-107 induced significant toxicity on normal hematopoiesis. To this end, six 2-month old C57BL/6j mice were treated with YX-2-107 at a daily dose of 150 mg/kg for 10 consecutive days. Mice did not display signs of distress or loss of weight during the treatment. Four days after termination of the treatment, mice were sacrificed, and peripheral blood and bone marrow cells were purified. Flow cytometry analysis of bone marrow cells showed no significant changes in the percentage of stem and progenitor cells and of B-cell precursors. Likewise, peripheral blood cell counts showed no effect of YX-2-107 on most cell subsets except for a moderate increase in the number of platelets and reticulocytes. (FIG. 24).

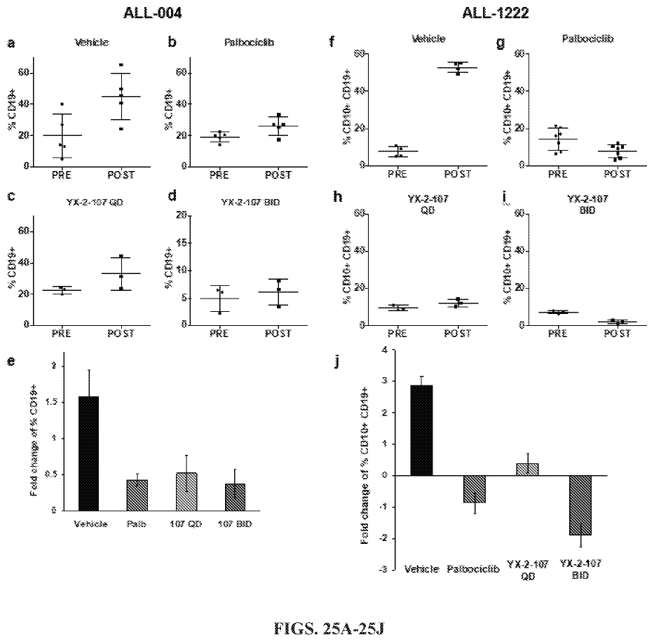

Since such relatively long treatment with YX-2-107 was well tolerated by normal mice, the effects of Palbociclib and YX-2-107 were compared on the peripheral blood leukemia load (% of CD19+CD10+ cells) of NSG mice injected with primary Ph+ ALL cells from two patients and then treated (10 consecutive days) with Palbociclib (150 mg/kg, by gavage), or YX-2-107 (125 or 150 mg/kg, i.p; once/day or twice/day at half dose/injection). As shown in FIG. 25, treatment with YX-2-107 is as effective as Palbociclib in suppressing peripheral blood leukemia burden. Of interest, the twice/day treatment with YX-2-107 appears to be more effective than the single-dose/day treatment regimen, at least in mice injected with ALL sample #1222, which is consistent with the pharmacokinetics of plasma exposure where the compound is cleared after about 4 hours. However, leukemia growth resumed rapidly upon cessation of treatment with either drug.

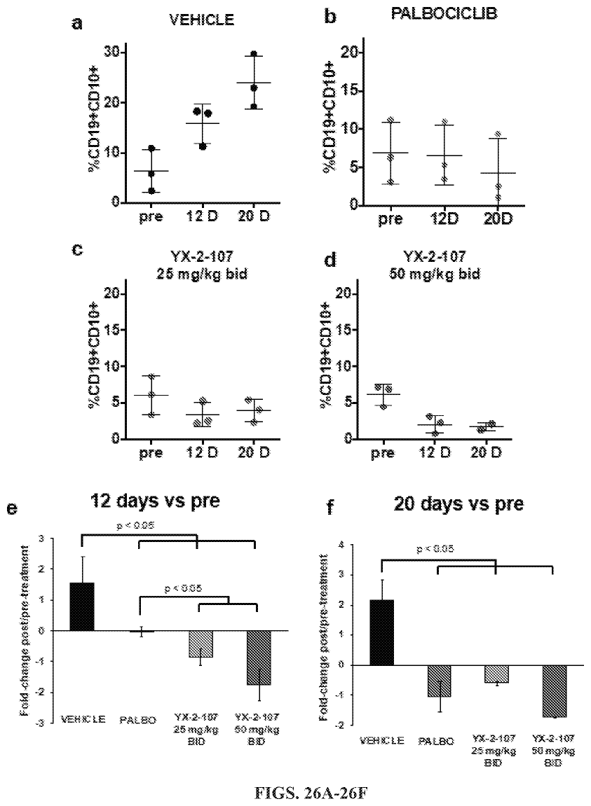

Lastly, peripheral blood leukemia load (% of CD19+CD10+ cells) was assessed of NRG-SGM3 mice (which produce human cytokines SCF, GM-CSF and IL-3 in the bone marrow niche) injected with a TKI-resistant (BCR-ABL1 T315I) primary Ph+ ALL sample and treated (20 consecutive days) with Palbociclib (150 mg/kg in the diet), or YX-2-107 (25 mg or 50 mg/kg twice/day, IP). As shown in FIG. 26, YX-2-107 appears to be significantly more effective than Palbociclib in suppressing the in vivo growth of this TKI-resistant Ph+ ALL after 12 or 20 days of treatment.

### D. Discussion

In this disclosure, the development of proteolysis-targeted chimeras (PROTACs) consisting of high-affinity small molecule ligands for CDK4/6 were prepared, and for the E3 ubiquitin ligase Cereblon, joined by linkers of different structure and/or size. Most Cereblon-recruting PROTACs were capable of selective degradation of CDK6 over CDK4 in Ph+ ALL cells. By contrast, PROTAC YX-2-233 which uses as an E3 ubiquitin ligase recruiter the MDM2 ligand RG7112 (Tovar et al, 2013) degraded CDK4 and CDK6 with equal efficiency. The selective degradation of CDK6 by Cereblon-recruiting PROTACs may be explained by formation of a ternary complex generating new protein-protein contacts that allow selective lysine ubiquitination of CDK6 over CDK4, followed by 26S proteasomal degradation.

The disclosure is consistent with findings from a recent study demonstrating the rapid formation in live cells of a ternary complex with CDK6 and Cereblon, but not with CDK4 and Cereblon, by a CDK4/6-targeted Cereblon-recruiting PROTAC (Brand et al., 2019). Selective degraders of CDK6 are attractive therapeutic agents in Ph+ ALL because Ph+ ALL cells are dependent for their growth on the expression of CDK6 whereas CDK4 function is dispensable (De Dominici et al., 2018). Moreover, CDK6 has kinase-independent growth-promoting effects (Fujimoto et al., 2007; Kollman et al., 2013; Scheicher et al., 2015; Buss et al., 2012; Handschick et al., 2014; Uras et al., 2019; Belluti et al., 2018) that can be exploited therapeutically by drugs that induce CDK6 degradation not by selective inhibitors of CDK6 kinase activity alone.

Indeed, CDK6-silenced Ph+ ALL cells are more susceptible to apoptosis and exhibit a slower disease progression in NSG mice than the Palbociclib-treated counterparts (FIG. 10), possibly as consequence of reduced expression of genes involved in cell survival, chromatin remodeling and mitochondrial metabolic pathways for energy production. Interestingly, the expression of CDK6 and HDAC1 was highly correlated in a dataset of 122 Ph+ ALL patient's samples (FIG. 14D), suggesting that the CDK6-HDAC1 pathway may be useful for the growth suppression/apoptosis of CDK6-silenced Ph+ ALL cells. It is also noteworthy that genetic or pharmacological inhibition of HDAC1 was reported to induce apoptosis of several B-ALL lines, although Ph+ cell lines were not among those tested (Stubbs et al., 2015). Together, these features of CDK6-silenced Ph+ cells support the idea that CDK6 degraders might be more effective therapeutic agents than CDK4/6 enzymatic inhibitors.

Among the present CDK6-degrading PROTACs, one compound, termed YX-2-107, was investigated in detail. In addition to its kinase-dependent effects (inhibition of phospho-RB and FOXM1 expression and S phase), this PROTAC was able to promote the preferential degradation of CDK6 over CDK4 in Ph+ ALL cells. In normal CD34+ human hematopoietic progenitors which depend for their proliferation on the expression/activity of both CDK4 and CDK6 (FIG. 20I), treatment with PROTAC YX-2-107 induced the selective degradation of CDK6 but did not inhibit significantly the S phase of these cells (FIGS. 20F & 20H), implying that, in the clinic, selective CDK6 degraders would be less toxic for normal hematopoietic cells than CDK4/6 dual enzymatic inhibitors. In this regard, neutropenia was the most common adverse-event (60-70%) in estrogen receptor (ER)+ breast cancer patients treated with dual CDK4/6 inhibitor Pabociclib or Ribociclib (Turner et al., 2018; Im et al., 2019). Such adverse-event would probably be clinically relevant in patients with acute leukemia in whom normal white blood cell counts are typically low due to the bone marrow replacement by leukemic cells, emphasizing the importance of using a selective CDK6 inhibitor to spare normal hematopoietic progenitors.

Similar to Palbociclib, YX-2-107 exhibited a relatively long half-life when incubated in mouse liver microsomes, which is expected to correlate with potent in vivo activity. However, YX-2-107 had a half-life of 1 h in a mouse PK study (FIG. 22B) when administered by IP, suggesting that further improvement in PK is warranted.

A short-term treatment of NSG mice with progressing Ph+ ALL was sufficient to significantly inhibit the proportion of S phase cells in the bone marrow, to markedly suppress the expression of the CDK4/6 substrates phospho-RB and FOXM1, and to induce the preferential degradation of CDK6 over CDK4. A long-term (2-3 weeks) treatment of NSG mice injected with de novo or TKI-resistant primary Ph+ ALL induced a marked suppression of peripheral blood leukemia load that was comparable or even superior to that induced by treatment with Palbociclib. PROTAC YX-2-107 was also as effective or superior to Palbociclib in suppressing the growth of a TKI-resistant Ph+ ALL in human cytokine-expressing NRG mice. This finding suggests that neither pharmacological resistance to TKIs, nor exposure to cytokines present in the human microenvironment impairs the therapeutic effects of PROTAC-based CDK6 degraders. Of interest, normal mice treated for 10 consecutive days with a high-dose (150 mg/Kg) of YX-2-107 did not exhibit significant changes in normal hematopoietic progenitors and mature cells (FIG. 24), consistent with the ex vivo findings showing no significant effect on the S phase of YX-2-107-treated human CD34+ cells (FIG. 20H).

These in vivo effects may represent the first demonstration of therapeutic efficacy by a PROTAC that targets an oncogenic protein which exerts kinase-dependent and independent effects. YX-2-107 may not possess the optimal in vitro, ex vivo, and in vivo properties to replace current enzymatic inhibitors of CDK4/6; nevertheless, derivatives of PROTAC YX-2-107 have been generated that are more potent in inhibiting S phase and in inducing CDK6 degradation or possess better metabolic stability. Further development is expected to yield second-generation PROTACs combining superior ex vivo activities (inhibition of CDK6-regulated S phase; CDK6 degradation) with improved pharmacokinetic properties that might establish these drugs as bona fide anti-cancer therapeutics with novel mechanisms of action.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Anderson, Practical Process Research & Development—A Guide for Organic Chemists, 2^{nd} ed., Academic Press, New York, 2012.
Handbook of Pharmaceutical Salts: Properties, and Use, Stahl and Wermuth Eds., Verlag Helvetica Chimica Acta, 2002.
Reagan-Shaw et al., FASEB J., 22(3):659-661, 2008.
Smith, March's Advanced Organic Chemistry: Reactions, Mechanisms, and Structure, 7^{th} Ed., Wiley, 2013.
Anders et al., Cancer Cell, 20:620-634, 2011.
Baughn et al., Cancer Res, 66:7661-7667, 2006.
Bellutti et al., Cancer Discov., 8: 884-897, 2018.
Brand et al., Cell Chem. Biol., 26:300-306, 2019.
Buckley et al., J. Am. Chem. Soc., 134, 4465-4468, 2012.
Bujnicki et al., Leukemia, 26: 615-622, 2012.
Bulatov and Ciulli, Biochem J., 467:365-368, 2015.
Burslem and Crews, Chem. Rev., 217:11269-11301, 2017.
Buss et al., PLoS One, 7(12):e51847, 2012.
Calabretta et al. Proc. Natl. Acad. Sci. USA, 88: 2351-2355, 1991.
Chae et al., Cancer Cell., 30:257-272, 2016.
Chen et al., Mol. Cancer Ther., 15:2273-2281, 2016.
Cox and Mann, Nat. Biotechnol., 26:1367-2372, 2008.
De Dominici et al., Cancer Res., 78: 1097-1109, 2018.
Drabsch et al., Proc. Natl. Acad. Sci. USA, 104:13762-13767, 2008.
Faderl et al., Blood, 91: 3995-4015, 1998.
Fielding et al., Blood, 123:843-50, 2014.
Fujimoto et al., EMBO J., 26: 2361-2370, 2007.
Galdeano et al., J. Am. Chem. Soc. 134:4465-4468, 2012.
Galdeano et al., J. Med. Chem., 57:8657-8663, 2014.
Guzman et al., Blood, 105: 4163-4169, 2005.
Handschick et al., Mol Cell. 53:193-208, 2014.
Hofmann et al., Blood, 99: 18860-1862, 2002.
Im et al., N. Engl J. Med., 381(4):307-316, 2019.
Kawasaki et al., The Prostate, 69: 827-837, 2009.
Kollmann et al., Cancer Cell, 24: 167-181, 2013.
Koo et al., Biochim. Biophys. Acta, 1819:28-37, 2012.
Lahortiga et al., Nat Genet., 39: 593-595, 2007.
Lai and Crews, Nat. Rev. Drug Discov., 16:101-114, 2017.
Laoukili et al., Nat. Cell Biol., 7:126-136, 2005.
Lee et al., Biochem. Pharmacol. 72: 1311-1321, 2006.
Leone et al., Genes Dev., 12:2120-2130, 1998.
Li et al., Cancer Cell, 34:893-905, 2018.
Lidonnici et al., Blood, 111: 4771-4779, 2008.
Lu and Schulze-Gahmen, J. Med. Chem., 49:3826-3831, 2006.
Lugo et al., Science, 247:1079-1082, 1990.
Matyskiela et al., J. Med. Chem., 61:535-542, 2018.
Metzger et al., J. Cell Sci., 125:531-537, 2012.
Minieri et al., Cancer Res., 78:5793-5807, 2018.
Neklesa et al., Pharmacol Ther., 174:138-144, 2017.
Pegoraro et al., J. Natl. Cancer. Inst., 70: 447-453, 1983.
Pfeifer et al., Blood, 110: 727-734, 2007.
Robinson and Ardley, J. Cell Sci., 117:5191-5194, 2004.
Sato et al., Bioorg. Med. Chem. Lett., 18:3354, 2008.
Scheicher et al., Blood, 125:90-101, 2015.
Sherr et al., Cancer Discov. 6: 353-367, 2016.
Short et al., Blood, 128:504-7, 2016.
Souers et al., Nat Med., 19:202-208, 2013.
Soverini et al., Cancer, 120:1002-9, 2014.
Spofford et al., J Biol Chem., 281: 25644-51, 2006.
Stubbs et al., Clin. Cancer Res., 21:2348-2358, 2015.
Tovar et al., Cancer Res., 73:2587-2597, 2013.
Turner et al., N. Engl J. Med., 379(20):1926-1936, 2018.
Uras et al., Blood. 133:1677-1690, 2019.
Uttarkar et al., Blood, 127: 1173-1182, 2016.
Uttarkar et al., Mol. Cancer Ther. 14: 1276-1285, 2015.
Vu et al., ACS Med Chem Lett., 4: 466-469, 2013.
Wada et al., Biochem Biophys Res Commun., 477:388-94, 2016.
Waldron et al., Leukemia, 26: 644-653, 2012.
Wang et al., Eur. J. Med. Chem., 122:546-556, 2016.
Wang et al., Nature, 546, 426-430, 2017.
Winter et al., Science, 348:1376-1380, 2015.
Wurz et al., J. Med. Chem., 61(2):453-461, 2018.
Xu et al., Cancer Cell, 33: 13-28, 2018.
Yang et al., Cancer Res., 66: 4758-4765, 2006.
Yang et al., Oncogene, 36:2255-2264, 2017.
Zhou et al., Breast Cancer Research Treatment, 115: 269-277, 2009.
Zuber et al., Genes Dev. 25: 1628-1640, 2011.

What is claimed is:

1. A compound of the formula: 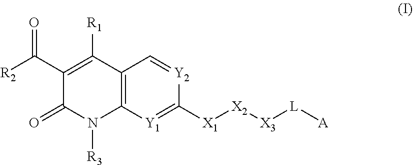
wherein:
R₁ is alkyl_{(C≤12)}, cycloalkyl_{(C≤12)}, aryl_{(C≤12)}, or a substituted version of any of these groups;
R₂ is alkyl_{(C≤12)}, cycloalkyl_{(C≤12)}, or a substituted version of either of these groups;
R₃ is cycloalkyl_{(C≤12)}, aryl_{(C≤12)}, or a substituted version of either of these groups;
Y₁ and Y₂ are each independently N or CH;
X₁ is O, S, or NRₐ,
Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:
—(C(O))_{d}(CH₂)ₐX₄—(CH₂)_{b}—Y₃—X₅—(CH₂)_{c}— (IA)
wherein:
d is 0 or 1;
a, b, or c is 0, 1, 2, 3, 4, 5, or 6;
X₄ is —C(O)—, —NR_{b}—, heteroarenediyl_{(C≤12)} or substituted heteroarenediyl_{(C≤12)};
wherein R_{b} and R_{c} are each independently selected from hydrogen, alkyl_{(C≤6)}, or substituted alkyl_{(C≤6)};
X₅ is —C(O)—, —NR_{b}—, —C(O)NR_{c}—, alkanediyl_{(C≤12)}, substituted alkanediyl_{(C≤12)}, arenediyl_{(C≤12)}, substituted arenediyl_{(C≤12)}, heteroarenediyl_{(C≤12)}, or substituted heteroarenediyl_{(C≤12)};
wherein R_{b} and R_{c} are each independently selected from hydrogen, alkyl_{(C≤6)}, or substituted alkyl_{(C≤6)};
Y₃ is a covalent bond, alkanediyl_{(C≤12)}, substituted alkanediyl_{(C≤12)}, —(CH₂CH₂O)ₑ(CH₂)_{f}—, —C(O)NR_{d}-alkanediyl_{(C≤12)}, or substituted —C(O)NR_{d}-alkanediyl_{(C≤12)};
wherein:
e is 1, 2, 3, 4, or 5;
f is 0, 1, 2, 3, 4, or 5; and
R_{d} is hydrogen, alkyl_{(C≤6)}, or substituted alkyl_{(C≤6)}; or
a linking group of the formula:
-(AA₁)ₓ- (IB)
wherein:
AA₁ is an amino acid residue; and
x is 1, 2, 3, 4, 5, or 6; and
A is hydrogen or an E3 ligase ligand; or
a compound of the formula: 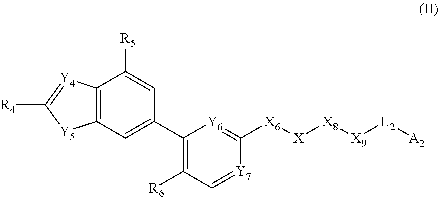
wherein:
R₄ is hydrogen, alkyl_{(C≤12)}, substituted alkyl_{(C≤12)}, cycloalkyl_{(C≤12)}, or substituted cycloalkyl_{(C≤12)};
R₅ and R₆ are each independently is hydrogen, halo, alkyl_{(C≤12)}, substituted alkyl_{(C≤12)}, cycloalkyl_{(C≤12)}, or substituted cycloalkyl_{(C≤12)};
Y₄, Y₆, and Y₇ are each independently N or CH;
Y₅ is O, S, or NR_{d}, wherein:
R_{d} is hydrogen, alkyl_{(C≤12)}, substituted alkyl_{(C≤12)}, cycloalkyl_{(C≤12)}, or substituted cycloalkyl_{(C≤12)};
X₆ is O, S, or NRₑ,
Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:
—(CH₂)_{g}X₁₀—(CH₂)ₕ—Y₈—X₁₁—(CH₂)ᵢ— (IIA)
wherein:
g, h, and i are each independently 0, 1, 2, 3, 4, or 5;
X₁₀ is —C(O)—, —NR_{f}—, heteroarenediyl_{(C≤12)} or substituted heteroarenediyl_{(C≤12)}, wherein:
R_{f} is hydrogen, alkyl_{(C≤6)}, or substituted alkyl_{(C≤6)};
X₁₁ is —C(O)—, —NR_{f}—, —C(O)NR_{g}—, heteroarenediyl_{(C≤12)} or substituted heteroarenediyl_{(C≤12)};
wherein R_{f} and R_{g} are each independently selected from hydrogen, alkyl_{(C≤6)}, or substituted alkyl_{(C≤6)};
Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
wherein:
j is 1, 2, 3, 4, or 5;
k is 0, 1, 2, 3, 4, or 5; and
R_{g} is hydrogen, alkyl_{(C≤6)}, or substituted alkyl_{(C≤6)}; or
a linking group of the formula:
-(AA₂)_{y}- (IIB)
wherein:
AA₂ is an amino acid residue; and
y is 1, 2, 3, 4, 5, or 6; and
A₂ is hydrogen or an E3 ligase ligand;
or a pharmaceutically acceptable salt of either of these formulae.

2. The compound of claim 1 further defined as: 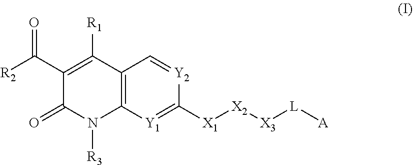
wherein:
R₁ is alkyl_{(C≤12)}, cycloalkyl_{(C≤12)}, aryl_{(C≤12)}, or a substituted version of any of these groups;
R₂ is alkyl_{(C≤12)}, cycloalkyl_{(C≤12)}, or a substituted version of either of these groups;
R₃ is cycloalkyl_{(C≤12)}, aryl_{(C≤12)}, or a substituted version of either of these groups;
Y₁ and Y₂ are each independently N or CH;
X₁ is O, S, or NRₐ,
Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:
—(C(O))_{d}(CH₂)ₐX₄—(CH₂)_{b}—Y₃—X₅—(CH₂)_{c}— (IA)
wherein:
d is 0 or 1;
a, b, or c is 0, 1, 2, 3, 4, 5, or 6;
X₄ is —C(O)—, —NR_{b}—, heteroarenediyl_{(C≤12)} or substituted heteroarenediyl_{(C≤12)};
wherein R_{b} and R_{c} are each independently selected from hydrogen, alkyl_{(C≤6)}, or substituted alkyl_{(C≤6)};
X₅ is —C(O)—, —NR_{b}—, —C(O)NR_{c}—, alkanediyl_{(C≤12)}, substituted alkanediyl_{(C≤12)}, arenediyl_{(C≤12)}, substituted arenediyl_{(C≤12)}, heteroarenediyl_{(C≤12)}, or substituted heteroarenediyl_{(C≤12)};
wherein R_{b} and R_{c} are each independently selected from hydrogen, alkyl_{(C≤6)}, or substituted alkyl_{(C≤6)};
Y₃ is a covalent bond, alkanediyl_{(C≤12)}, substituted alkanediyl_{(C≤12)}, —(CH₂CH₂O)ₑ(CH₂)_{f}—, —C(O)NR_{d}-alkanediyl_{(C≤12)}, or substituted —C(O)NR_{d}-alkanediyl_{(C≤12)};
wherein:
e is 1, 2, 3, 4, or 5;
f is 0, 1, 2, 3, 4, or 5; and
R_{d} is hydrogen, alkyl_{(C≤6)}, or substituted alkyl_{(C≤6)}; or
a linking group of the formula:
-(AA₁)ₓ- (IB)
wherein:
AA₁ is an amino acid residue; and
x is 1, 2, 3, 4, 5, or 6; and
A is hydrogen or an E3 ligase ligand;
or a pharmaceutically acceptable salt thereof.

3. The compound of claim 1 further defined as: 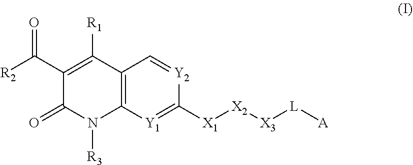
wherein:
R₁ is alkyl_{(C≤12)}, cycloalkyl_{(C≤12)}, aryl_{(C≤12)}, or a substituted version of any of these groups;
R₂ is alkyl_{(C≤12)}, cycloalkyl_{(C≤12)}, or a substituted version of either of these groups;
R₃ is cycloalkyl_{(C≤12)}, aryl_{(C≤12)}, or a substituted version of either of these groups;
Y₁ and Y₂ are each independently N or CH;
X₁ is O, S, or NRₐ,
Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:
—C(O)(CH₂)ₐX₄—(CH₂)_{b}—Y₃—X₅—(CH₂)_{c}— (IC)
wherein:
a, b, or c is 0, 1, 2, 3, 4, or 5;
X₄ is —C(O)—, —NR_{b}—, heteroarenediyl_{(C≤12)} or substituted heteroarenediyl_{(C≤12)};
wherein R_{b} and R_{c} are each independently selected from hydrogen, alkyl_{(C≤6)}, or substituted alkyl_{(C≤6)};
X₅ is —C(O)—, —NR_{b}—, —C(O)NR_{c}—, heteroarenediyl_{(C≤12)} or substituted heteroarenediyl_{(C≤12)};
wherein R_{b} and R_{c} are each independently selected from hydrogen, alkyl_{(C≤6)}, or substituted alkyl_{(C≤6)};
Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
wherein:
d is 1, 2, 3, 4, or 5;
e is 0, 1, 2, 3, 4, or 5; and
R_{d} is hydrogen, alkyl_{(C≤6)}, or substituted alkyl_{(C≤6)}; or
a linking group of the formula:
-(AA₁)ₓ- (IB)
wherein:
AA₁ is an amino acid residue; and
x is 1, 2, 3, 4, 5, or 6; and
A is an E3 ligase ligand;
or a pharmaceutically acceptable salt thereof.

4. The compound of claim 3, wherein the compound is further defined as: 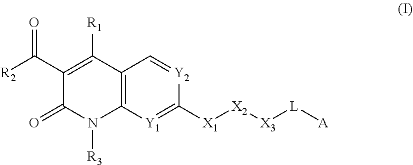
wherein:
R₁ is alkyl_{(C≤12)}, cycloalkyl_{(C≤12)}, aryl_{(C≤12)}, or a substituted version of any of these groups;
R₂ is alkyl_{(C≤12)}, cycloalkyl_{(C≤12)}, or a substituted version of either of these groups;
R₃ is cycloalkyl_{(C≤12)}, aryl_{(C≤12)}, or a substituted version of either of these groups;
Y₁ and Y₂ are each independently N or CH;
X₁ is O, S, or NRₐ,
Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:
—C(O)(CH₂)ₐX₄—(CH₂)_{b}—Y₃—X₅—(CH₂)_{c}— (IC)
wherein:
a, b, or c is 0, 1, 2, 3, 4, or 5;
X₄ is —C(O)—, —NR_{b}—, heteroarenediyl_{(C≤12)} or substituted heteroarenediyl_{(C≤12)};
wherein R_{b} and R_{c} are each independently selected from hydrogen, alkyl_{(C≤6)}, or substituted alkyl_{(C≤6)};
X₅ is —C(O)—, —NR_{b}—, —C(O)NR_{c}—, heteroarenediyl_{(C≤12)} or substituted heteroarenediyl_{(C≤12)};
wherein R_{b} and R_{c} are each independently selected from hydrogen, alkyl_{(C≤6)}, or substituted alkyl_{(C≤6)};
Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
wherein:
d is 1, 2, 3, 4, or 5;
e is 0, 1, 2, 3, 4, or 5; and
R_{d} is hydrogen, alkyl_{(C≤6)}, or substituted alkyl_{(C≤6)}; and
A is an E3 ligase ligand;
or a pharmaceutically acceptable salt thereof.

5. The compound of either claim 3 or claim 4, wherein the compound is further defined as: 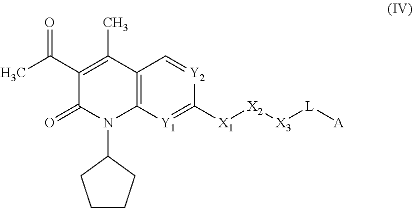
wherein:
Y₁ and Y₂ are each independently N or CH;
X₁ is O, S, or NRₐ,
Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:
—C(O)(CH₂)ₐX₄—(CH₂)_{b}—Y₃—X₅—(CH₂)_{c}— (IC)
wherein:
a, b, or c is 0, 1, 2, 3, 4, or 5;
X₄ is —C(O)—, —NR_{b}—, heteroarenediyl_{(C≤12)} or substituted heteroarenediyl_{(C≤12)};
wherein R_{b} and R_{c} are each independently selected from hydrogen, alkyl_{(C≤6)}, or substituted alkyl_{(C≤6)};
X₅ is —C(O)—, —NR_{b}—, —C(O)NR_{c}—, heteroarenediyl_{(C≤12)} or substituted heteroarenediyl_{(C≤12)};
wherein R_{b} and R_{c} are each independently selected from hydrogen, alkyl_{(C≤6)}, or substituted alkyl_{(C≤6)};
Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
wherein:
d is 1, 2, 3, 4, or 5;
e is 0, 1, 2, 3, 4, or 5; and
R_{d} is hydrogen, alkyl_{(C≤6)}, or substituted alkyl_{(C≤6)}; and
A is an E3 ligase ligand;
or a pharmaceutically acceptable salt thereof.

6. The compound according to any one of claims 1-5, wherein the compound is further defined as: 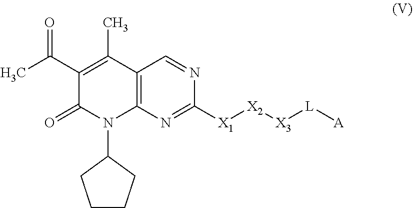
wherein:
X₁ is O, S, or NRₐ,
Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:
—C(O)(CH₂)ₐX₄—(CH₂)_{b}—Y₃—X₅—(CH₂)_{c}— (IC)
wherein:
a, b, or c is 0, 1, 2, 3, 4, or 5;
X₄ is —C(O)—, —NR_{b}—, heteroarenediyl_{(C≤12)} or substituted heteroarenediyl_{(C≤12)};
wherein R_{b} and R_{c} are each independently selected from hydrogen, alkyl_{(C≤6)}, or substituted alkyl_{(C≤6)};
X₅ is —C(O)—, —NR_{b}—, or —C(O)NR_{c}—;
wherein R_{b} and R_{c} are each independently selected from hydrogen, alkyl_{(C≤6)}, or substituted alkyl_{(C≤6)};
Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
wherein:
d is 1, 2, 3, 4, or 5;
e is 0, 1, 2, 3, 4, or 5; and
R_{d} is hydrogen, alkyl_{(C≤6)}, or substituted alkyl_{(C≤6)}; and
A is an E3 ligase ligand;
or a pharmaceutically acceptable salt thereof.

7. The compound according to any one of claims 1-6, wherein the compound is further defined as: 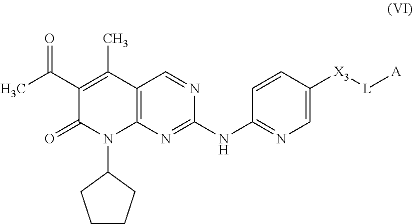
wherein:
X₃ is heterocycloalkanediyl_{(C≤12)} or substituted heterocycloalkanediyl_{(C≤12)};
L is a linking group of the formula:
—C(O)(CH₂)ₐX₄—(CH₂)_{b}—Y₃—X₅—(CH₂)_{c}— (IC)
wherein:
a, b, or c is 0, 1, 2, 3, 4, or 5;
X₄ is —C(O)—, —NR_{b}—, heteroarenediyl_{(C≤12)} or substituted heteroarenediyl_{(C≤12)};
wherein R_{b} and R_{c} are each independently selected from hydrogen, alkyl_{(C≤6)}, or substituted alkyl_{(C≤6)};
X₅ is —C(O)—, —NR_{b}—, or —C(O)NR_{c}—;
wherein R_{b} and R_{c} are each independently selected from hydrogen, alkyl_{(C≤6)}, or substituted alkyl_{(C≤6)};
Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
wherein:
d is 1, 2, 3, 4, or 5;
e is 0, 1, 2, 3, 4, or 5; and
R_{d} is hydrogen, alkyl_{(C≤6)}, or substituted alkyl_{(C≤6)}; and
A is an E3 ligase ligand;
or a pharmaceutically acceptable salt thereof.

8. The compound according to any one of claims 1-7, wherein the compound is further defined as: 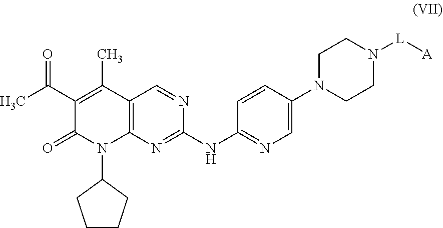
wherein:
L is a linking group of the formula:
—C(O)—(CH₂)ₐX₄—(CH₂)_{b}—Y₃—X₅—(CH₂)_{c}— (IC)
wherein:
a, b, or c is 0, 1, 2, 3, 4, or 5; provided the sum of a, b, and c are greater than 1;
X₄ is —C(O)—, —NR_{b}—, heteroarenediyl_{(C≤12)} or substituted heteroarenediyl_{(C≤12)};
wherein R_{b} and R_{c} are each independently selected from hydrogen, alkyl_{(C≤6)}, or substituted alkyl_{(C≤6)};
X₅ is —C(O)—, —NR_{b}—, or —C(O)NR_{c}—;
wherein R_{b} and R_{c} are each independently selected from hydrogen, alkyl_{(C≤6)}, or substituted alkyl_{(C≤6)};
Y₃ is alkanediyl_{(C≤12)}, substituted alkanediyl_{(C≤12)}, —(CH₂CH₂O)_{d}(CH₂)ₑ—, —C(O)NR_{d}-alkanediyl_{(C≤12)}, or substituted —C(O)NR_{d}-alkanediyl_{(C≤12)};
wherein:
d is 1, 2, 3, 4, or 5;
e is 0, 1, 2, 3, 4, or 5; and
R_{d} is hydrogen, alkyl_{(C≤6)}, or substituted alkyl_{(C≤6)};
A is an E3 ligase ligand;
or a pharmaceutically acceptable salt thereof.

9. The compound according to any one of claims 1-3, wherein the compound is further defined as: 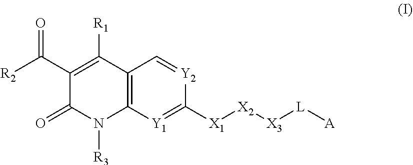
wherein:
R₁ is alkyl_{(C≤12)}, cycloalkyl_{(C≤12)}, aryl_{(C≤12)}, or a substituted version of any of these groups;
R₂ is alkyl_{(C≤12)}, cycloalkyl_{(C≤12)}, or a substituted version of either of these groups;
R₃ is cycloalkyl_{(C≤12)}, aryl_{(C≤12)}, or a substituted version of either of these groups;
Y₁ and Y₂ are each independently N or CH;
X₁ is O, S, or NRₐ,
Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:
-(AA₁)ₓ- (IB)
wherein:
AA₁ is an amino acid residue; and
x is 1, 2, 3, 4, 5, or 6;
A is an E3 ligase ligand;
or a pharmaceutically acceptable salt thereof.

10. The compound according to any one of claims 1-3 and 9, wherein the compound is further defined as: 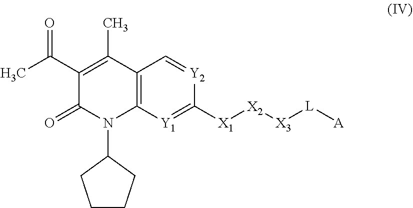
wherein:
Y₁ and Y₂ are each independently N or CH;
X₁ is O, S, or NRₐ, Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:
-(AA₁)ₓ- (IB)
wherein:
AA₁ is an amino acid residue; and
x is 1, 2, 3, 4, 5, or 6;
A is an E3 ligase ligand;
or a pharmaceutically acceptable salt thereof.

11. The compound according to any one of claims 1-3, 9, and 10, wherein the compound is further defined as: 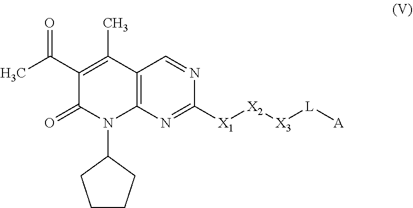
wherein:
X₁ is O, S, or NRₐ,
Rₐ is hydrogen, alkyl_{(C≤6)}, or substituted alkyl_{(C≤6)};
X₂ is heteroarenediyl_{(C≤12)} or substituted heteroarenediyl_{(C≤12)};
X₃ is heterocycloalkanediyl_{(C≤12)} or substituted heterocycloalkanediyl_{(C≤12)};
L is a linking group of the formula:
-(AA₁)ₓ- (IB)
wherein:
AA₁ is an amino acid residue; and
x is 1, 2, 3, 4, 5, or 6;
A is an E3 ligase ligand;
or a pharmaceutically acceptable salt thereof.

12. The compound according to any one of claims 1-3 and 9-11, wherein the compound is further defined as: 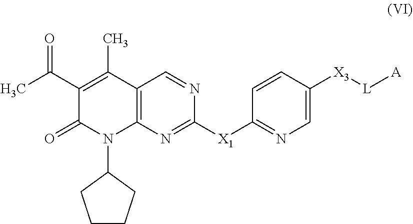
wherein:
X₃ is heterocycloalkanediyl_{(C≤12)} or substituted heterocycloalkanediyl_{(C≤12)};
L is a linking group of the formula:
-(AA₁)ₓ- (IB)
wherein:
AA₁ is an amino acid residue; and
x is 1, 2, 3, 4, 5, or 6;
A is an E3 ligase ligand;
or a pharmaceutically acceptable salt thereof.

13. The compound according to any one of claims 1-3 and 9-12, wherein the compound is further defined as: 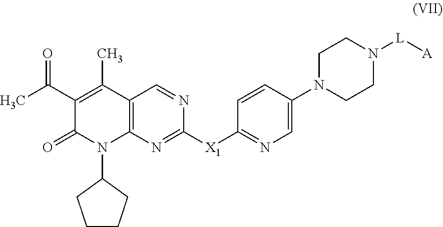
wherein:
L is a linking group of the formula:
-(AA₁)ₓ- (IB)
wherein:
AA₁ is an amino acid residue; and
x is 1, 2, 3, 4, 5, or 6;
A is an E3 ligase ligand;
or a pharmaceutically acceptable salt thereof.

14. The compound of claim 1, further defined as: 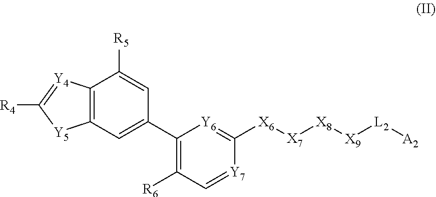
wherein:
R₄ is hydrogen, alkyl_{(C≤12)}, substituted alkyl_{(C≤12)}, cycloalkyl_{(C≤12)}, or substituted cycloalkyl_{(C≤12)};
R₅ and R₆ are each independently is hydrogen, halo, alkyl_{(C≤12)}, substituted alkyl_{(C≤12)}, cycloalkyl_{(C≤12)}, or substituted cycloalkyl_{(C≤12)};
Y₄, Y₆, and Y₇ are each independently N or CH;
Y₅ is O, S, or NR_{d}, wherein:
R_{d} is hydrogen, alkyl_{(C≤12)}, substituted alkyl_{(C≤12)}, cycloalkyl_{(C≤12)}, or substituted cycloalkyl_{(C≤12)};
X₆ is O, S, or NRₑ,
Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:
—(CH₂)_{g}X₁₀—(CH₂)ₕ—Y₈—X₁₁—(CH₂)ᵢ— (IIA)
wherein:
g, h, and i are each independently 0, 1, 2, 3, 4, or 5;
X₁₀ is —C(O)—, —NR_{f}—, heteroarenediyl_{(C≤12)} or substituted heteroarenediyl_{(C≤12)}, wherein:
R_{f} is hydrogen, alkyl_{(C≤6)}, or substituted alkyl_{(C≤6)};
X₁₁ is —C(O)—, —NR_{f}—, —C(O)NR_{g}—, heteroarenediyl_{(C≤12)} or substituted heteroarenediyl_{(C≤12)};
wherein R_{f} and R_{g} are each independently selected from hydrogen, alkyl_{(C≤6)}, or substituted alkyl_{(C≤6)};
Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
wherein:
j is 1, 2, 3, 4, or 5;
k is 0, 1, 2, 3, 4, or 5; and
R_{g} is hydrogen, alkyl_{(C≤6)}, or substituted alkyl_{(C≤6)}; or
a linking group of the formula:
-(AA₂)_{y}- (IIB)
wherein:
AA₂ is an amino acid residue; and
y is 1, 2, 3, 4, 5, or 6; and
A₂ is hydrogen or an E3 ligase ligand;
or a pharmaceutically acceptable salt thereof.

15. The compound of either claim 1 or claim 14 further defined as: 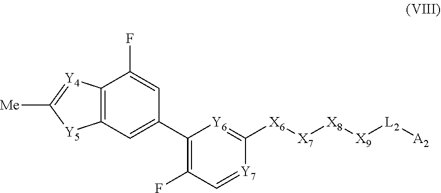
wherein:
Y₄, Y₆, and Y₇ are each independently N or CH;
Y₅ is O, S, or NR_{d}, wherein:
R_{d} is hydrogen, alkyl_{(C≤12)}, substituted alkyl_{(C≤12)}, cycloalkyl_{(C≤12)}, or substituted cycloalkyl_{(C≤12)};
X₆ is O, S, or NRₑ,
Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:
—(CH₂)_{g}X₁₀—(CH₂)ₕ—Y₈—X₁₁—(CH₂)ᵢ— (IIA)
wherein:
g, h, and i are each independently 0, 1, 2, 3, 4, or 5;
X₁₀ is —C(O)—, —NR_{f}—, heteroarenediyl_{(C≤12)} or substituted heteroarenediyl_{(C≤12)}, wherein:
R_{f} is hydrogen, alkyl_{(C≤6)}, or substituted alkyl_{(C≤6)};
X₁₁ is —C(O)—, —NR_{f}—, —C(O)NR_{g}—, heteroarenediyl_{(C≤12)} or substituted heteroarenediyl_{(C≤12)};
wherein R_{f} and R_{g} are each independently selected from hydrogen, alkyl_{(C≤6)}, or substituted alkyl_{(C≤6)};
Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
wherein:
j is 1, 2, 3, 4, or 5;
k is 0, 1, 2, 3, 4, or 5; and
R_{g} is hydrogen, alkyl_{(C≤6)}, or substituted alkyl_{(C≤6)}; or
a linking group of the formula:
-(AA₂)_{y}- (IIB)
wherein:
AA₂ is an amino acid residue; and
y is 1, 2, 3, 4, 5, or 6; and
A₂ is hydrogen or an E3 ligase ligand;
or a pharmaceutically acceptable salt thereof.

16. The compound according to any one of claims 1, 14, and 15 further defined as: 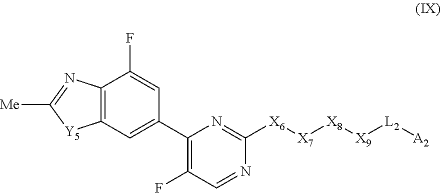
wherein:
Y₅ is O, S, or NR_{d}, wherein:
R_{d} is hydrogen, alkyl_{(C≤12)}, substituted alkyl_{(C≤12)}, cycloalkyl_{(C≤12)}, or substituted cycloalkyl_{(C≤12)};
X₆ is O, S, or NRₑ,
Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:
—(CH₂)_{g}X₁₀—(CH₂)ₕ—Y₈—X₁₁—(CH₂)ᵢ— (IIA)
wherein:
g, h, and i are each independently 0, 1, 2, 3, 4, or 5;
X₁₀ is —C(O)—, —NR_{f}—, heteroarenediyl_{(C≤12)} or substituted heteroarenediyl_{(C≤12)}, wherein:
R_{f} is hydrogen, alkyl_{(C≤6)}, or substituted alkyl_{(C≤6)};
X₁₁ is —C(O)—, —NR_{f}—, —C(O)NR_{g}—, heteroarenediyl_{(C≤12)} or substituted heteroarenediyl_{(C≤12)};
wherein R_{f} and R_{g} are each independently selected from hydrogen, alkyl_{(C≤6)}, or substituted alkyl_{(C≤6)};
Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
wherein:
j is 1, 2, 3, 4, or 5;
k is 0, 1, 2, 3, 4, or 5; and
R_{g} is hydrogen, alkyl_{(C≤6)}, or substituted alkyl_{(C≤6)}; or
a linking group of the formula:
-(AA₂)_{y}- (IIB)
wherein:
AA₂ is an amino acid residue; and
y is 1, 2, 3, 4, 5, or 6; and
A₂ is hydrogen or an E3 ligase ligand;
or a pharmaceutically acceptable salt thereof.

17. The compound according to any one of claims 1 and 14-16 further defined as: 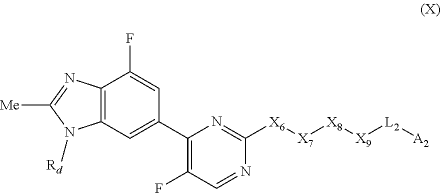
wherein:
R_{d} is hydrogen, alkyl_{(C≤12)}, substituted alkyl_{(C≤12)}, cycloalkyl_{(C≤12)}, or substituted cycloalkyl_{(C≤12)};
X₆ is O, S, or NRₑ,
Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:
—(CH₂)_{g}X₁₀—(CH₂)ₕ—Y₈—X₁₁—(CH₂)ᵢ— (IIA)
wherein:
g, h, and i are each independently 0, 1, 2, 3, 4, or 5;
X₁₀ is —C(O)—, —NR_{f}—, heteroarenediyl_{(C≤12)} or substituted heteroarenediyl_{(C≤12)}, wherein:
R_{f} is hydrogen, alkyl_{(C≤6)}, or substituted alkyl_{(C≤6)};
X₁₁ is —C(O)—, —NR_{f}—, —C(O)NR_{g}—, heteroarenediyl_{(C≤12)} or substituted heteroarenediyl_{(C≤12)};
wherein R_{f} and R_{g} are each independently selected from hydrogen, alkyl_{(C≤6)}, or substituted alkyl_{(C≤6)};
Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
wherein:
j is 1, 2, 3, 4, or 5;
k is 0, 1, 2, 3, 4, or 5; and
R_{g} is hydrogen, alkyl_{(C≤6)}, or substituted alkyl_{(C≤6)}; or
a linking group of the formula:
-(AA₂)_{y}- (IIB)
wherein:
AA₂ is an amino acid residue; and
y is 1, 2, 3, 4, 5, or 6; and
A₂ is hydrogen or an E3 ligase ligand;
or a pharmaceutically acceptable salt thereof.

18. The compound according to any one of claims 1 and 14-17 further defined as: 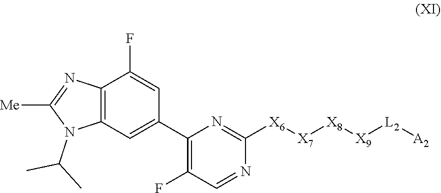
wherein:
X₆ is O, S, or NRₑ,
Rₑ is hydrogen, alkyl_{(C≤6)}, or substituted alkyl_{(C≤6)};
X₇ is heteroarenediyl_{(C≤12)} or substituted heteroarenediyl_{(C≤12)};
X₈ is alkanediyl_{(C≤12)} or substituted alkanediyl_{(C≤12)};
X₉ is heterocycloalkanediyl_{(C≤12)} or substituted heterocycloalkanediyl_{(C≤12)};
L₂ is a linking group of the formula:
—(CH₂)_{g}X₁₀—(CH₂)ₕ—Y₈—X₁₁—(CH₂)ᵢ— (IIA)
wherein:
g, h, and i are each independently 0, 1, 2, 3, 4, or 5;
X₁₀ is —C(O)—, —NR_{f}—, heteroarenediyl_{(C≤12)} or substituted heteroarenediyl_{(C≤12)}, wherein:
R_{f} is hydrogen, alkyl_{(C≤6)}, or substituted alkyl_{(C≤6)};
X₁₁ is —C(O)—, —NR_{f}—, —C(O)NR_{g}—, heteroarenediyl_{(C≤12)} or substituted heteroarenediyl_{(C≤12)};
wherein R_{f} and R_{g} are each independently selected from hydrogen, alkyl_{(C≤6)}, or substituted alkyl_{(C≤6)};
Y₈ is a covalent bond, alkanediyl_{(C≤12)}, substituted alkanediyl_{(C≤12)}, —(CH₂CH₂O)ⱼ(CH₂)ₖ—, —C(O)NR_{g}-alkanediyl_{(C≤12)}, or substituted —C(O)NR_{g}-alkanediyl_{(C≤12)};
wherein:
j is 1, 2, 3, 4, or 5;
k is 0, 1, 2, 3, 4, or 5; and
R_{g} is hydrogen, alkyl_{(C≤6)}, or substituted alkyl_{(C≤6)}; or
a linking group of the formula:
-(AA₂)_{y}- (IIB)
wherein:
AA₂ is an amino acid residue; and
y is 1, 2, 3, 4, 5, or 6; and
A₂ is hydrogen or an E3 ligase ligand;
or a pharmaceutically acceptable salt thereof.

19. The compound according to any one of claims 3-8, wherein a is 0, 1, 2, or 3.

20. The compound of claim 19, wherein a is 0 or 1.

21. The compound of claim 19, wherein a is 1 or 2.

22. The compound according to any one of claims 3-8, wherein a is 6.

23. The compound according to any one of claims 3-8 and 19-21, wherein b is 0, 1, 2, or 3.

24. The compound of claim 23, wherein b is 0 or 1.

25. The compound of claim 23, wherein b is 1 or 2.

26. The compound according to any one of claims 3-8 and 19-25, wherein c is 0, 1, 2, or 3.

27. The compound of claim 26, wherein c is 0 or 1.

28. The compound of claim 26, wherein c is 1 or 2.

29. The compound according to any one of claims 3-8 and 19-28, wherein d is 0.

30. The compound according to any one of claims 3-8 and 19-28, wherein d is 1.

31. The compound according to any one of claims 3-8 and 19-28, wherein X₄ is heteroarenediyl_{(C≤12)} or substituted heteroarenediyl_{(C≤12)}.

32. The compound of claim 31, wherein X₄ is 1,2,3-triazol-1,4-diyl.

33. The compound according to any one of claims 3-8 and 19-28, wherein X₄ is NR_{b}.

34. The compound of claim 33, wherein X₄ is NH or N(CH₃).

35. The compound according to any one of claims 3-8 and 19-34, wherein X₅ is —C(O)NR_{c}; wherein R_{c} is hydrogen, alkyl_{(C≤6)}, or substituted alkyl_{(C≤6)}.

36. The compound of claim 35, wherein X₅ is —C(O)NH—.

37. The compound according to any one of claims 3-8 and 19-34, wherein X₅ is —C(O)—.

38. The compound according to any one of claims 3-8 and 19-37, wherein Y₃ is a covalent bond.

39. The compound according to any one of claims 3-8 and 19-37, wherein Y₃ is alkanediyl_{(C≤8)} or substituted alkanediyl_{(C≤8)}.

40. The compound of claim 39, wherein Y₃ is methanediyl, ethanediyl, propanediyl, or butanediyl.

41. The compound according to any one of claims 3-8 and 19-37, wherein Y₃ is —C(O)NR_{d}-alkanediyl_{(C≤12)} or substituted —C(O)NR_{d}-alkanediyl_{(C≤12)}.

42. The compound of claim 41, wherein Y₃ is —C(O)NH-alkanediyl_{(C≤12)} or substituted —C(O)NH-alkanediyl_{(C≤12)}.

43. The compound of either claim 41 or claim 42, wherein the alkanediyl_{(C≤12)} or substituted alkanediyl_{(C≤12)} is methanediyl, ethanediyl, propanediyl, butanediyl, pentanediyl, or hexanediyl.

44. The compound according to any one of claims 3-8 and 19-37, wherein Y₃ is —(CH₂CH₂O)_{d}(CH₂)ₑ—, wherein: e is 1, 2, 3, 4, or 5; and f is 0, 1, 2, 3, 4, or 5.

45. The compound of claim 44, wherein e is 2, 3, or 4.

46. The compound of either claim 44 or claim 45, wherein f is 0 or 1.

47. The compound according to any one of claims 3 and 9-13, wherein AA₁ is a canonical amino acid.

48. The compound according to any one of claims 3, 9-13, and 47, wherein x is 1, 2, or 3.

49. The compound according to any one of claims 1 and 14-18, wherein X₆ is NRₑ, wherein Rₑ is hydrogen, alkyl_{(C≤6)}, or substituted alkyl_{(C≤6)}.

50. The compound according to any one of claims 1, 14-18, and 49, wherein X₇ is pyridinediyl.

51. The compound of claim 50, wherein X₇ is 2,5-pyridinediyl.

52. The compound according to any one of claims 1, 14-18, and 49-51, wherein X₈ is alkanediyl_{(C≤6)}.

53. The compound of claim 52, wherein X₈ is methylene.

54. The compound according to any one of claims 1, 14-18, and 48-53, wherein X₉ is heterocycloalkanediyl_{(C≤6)}.

55. The compound of claim 54, wherein X₉ is 1,4-piperazindiyl.

56. The compound according to any one of claims 1, 14-18, and 48-55, wherein g is 0, 1, or 2.

57. The compound of claim 56, wherein g is 2.

58. The compound according to any one of claims 1, 14-18, and 48-57, wherein h is 0, 1, or 2.

59. The compound of claim 58, wherein h is 0.

60. The compound according to any one of claims 1, 14-18, and 48-59, wherein i is 0, 1, or 2.

61. The compound of claim 60, wherein i is 1.

62. The compound according to any one of claims 1, 14-18, and 48-61, wherein X₁₀ is —NR_{f}—.

63. The compound of claim 62, wherein R_{f} is hydrogen.

64. The compound according to any one of claims 1, 14-18, and 48-63, wherein Y₈ is a covalent bond.

65. The compound according to any one of claims 1, 14-18, and 48-64, wherein X₁₁ is —C(O)—.

66. The compound according to either claim 1 or claim 2, wherein A is hydrogen.

67. The compound according to any one of claims 1-13 and 19-48, wherein A is an E3 ligase ligand for VHL, MDM2, cereblon, or cIAP.

68. The compound of claim 67, wherein the E3 ligase ligand is pomalidomide, thalidomide, lenalidomide, VHL-1, adamantane, 1-((4,4,5,5,5-pentafluoropentyl)sulfinyl)nonane, nutlin-3a, RG7112, RG7338, AMG 232, AA-115, bestatin, MV-1, LCL161, or a derivative thereof.

69. The compound according to any one of claims 14-18 and 49-55, wherein A₂ is hydrogen.

70. The compound according to any one of claims 14-18 and 49-55, wherein A₂ is an E3 ligase ligand for VHL, MDM2, cereblon, or cIAP.

71. The compound of claim 70, wherein the E3 ligase ligand is pomalidomide, thalidomide, lenalidomide, VHL-1, adamantane, 1-((4,4,5,5,5-pentafluoropentyl)sulfinyl)nonane, nutlin-3a, RG7112, RG7338, AMG 232, AA-115, bestatin, MV-1, LCL161, or a derivative thereof.

72. The compound according to any one of claims 67-71, wherein the E3 ligase ligand is:

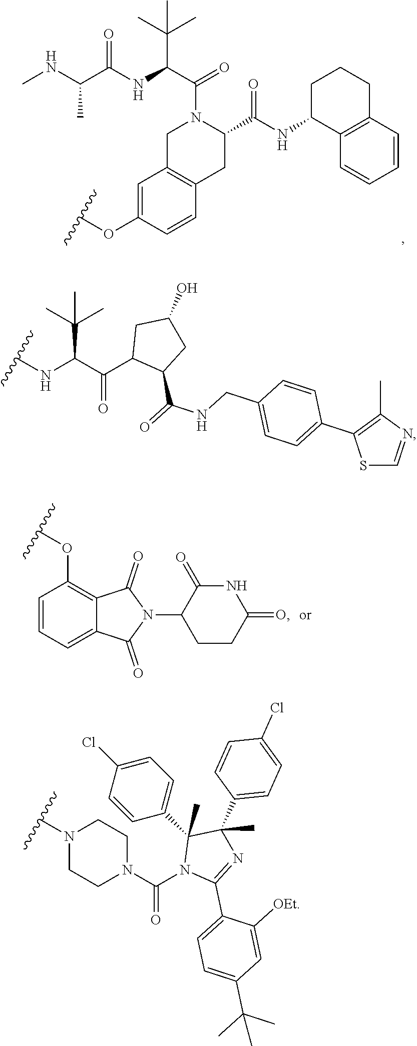 [SMILES: *Oc1ccc2c(c1)CN(C(=O)[C@@H](NC(=O)[C@H](C)NC)C(C)(C)C)[C@H](C(=O)N[C@@H]1CCCc3ccccc31)C2 ; *N[C@H](C(=O)C1C[C@H](O)C[C@H]1C(=O)NCc1ccc(-c2scnc2C)cc1)C(C)(C)C ; *Oc1cccc2c1C(=O)N(C1CCC(=O)NC1=O)C2=O ; *N1CCN(C(=O)N2C(c3ccc(C(C)(C)C)cc3OCC)=N[C@@](C)(c3ccc(Cl)cc3)[C@@]2(C)c2ccc(Cl)cc2)CC1]

73. The compound according to any one of claims 1-72, wherein the compound is further defined as: 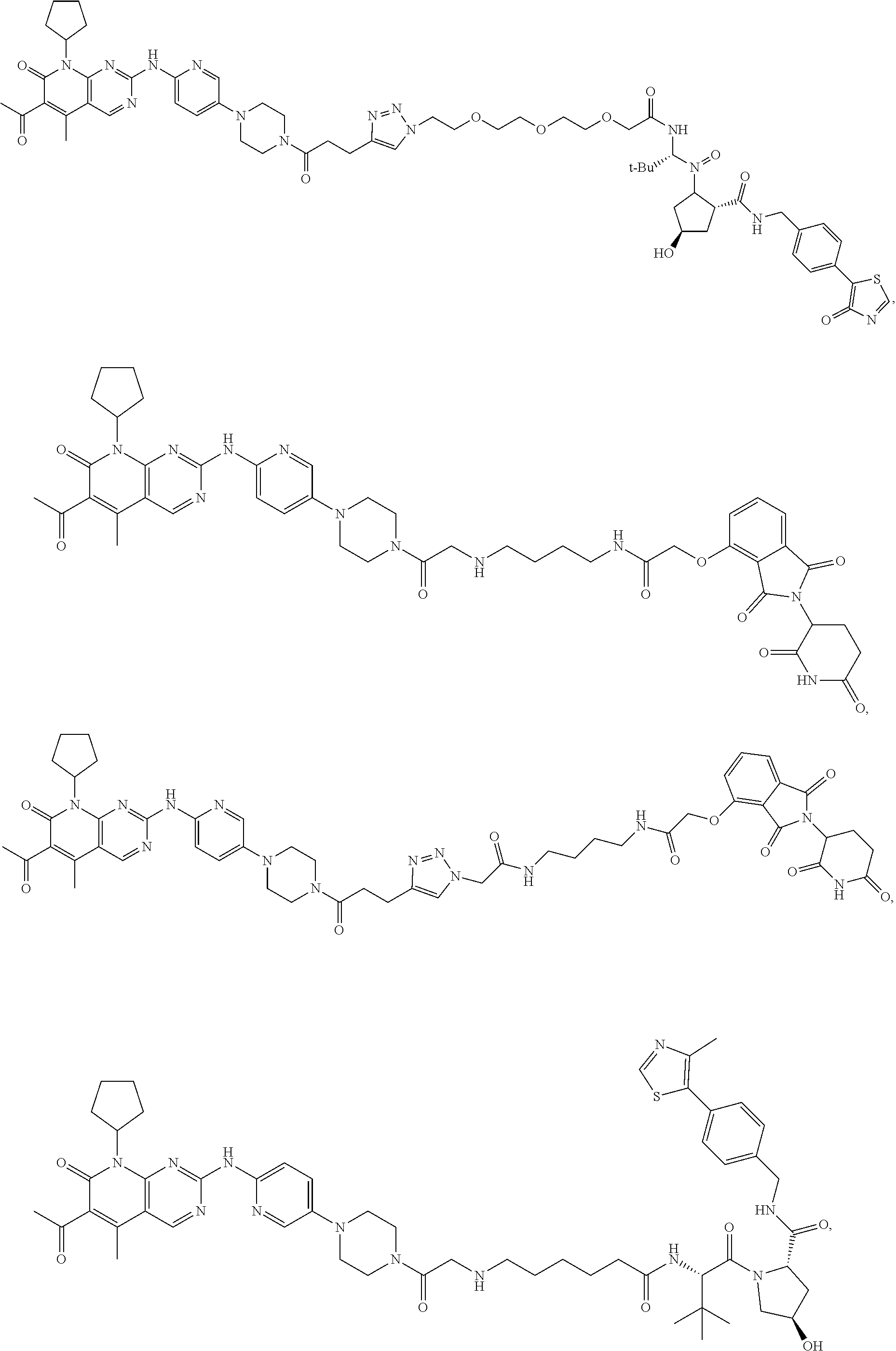 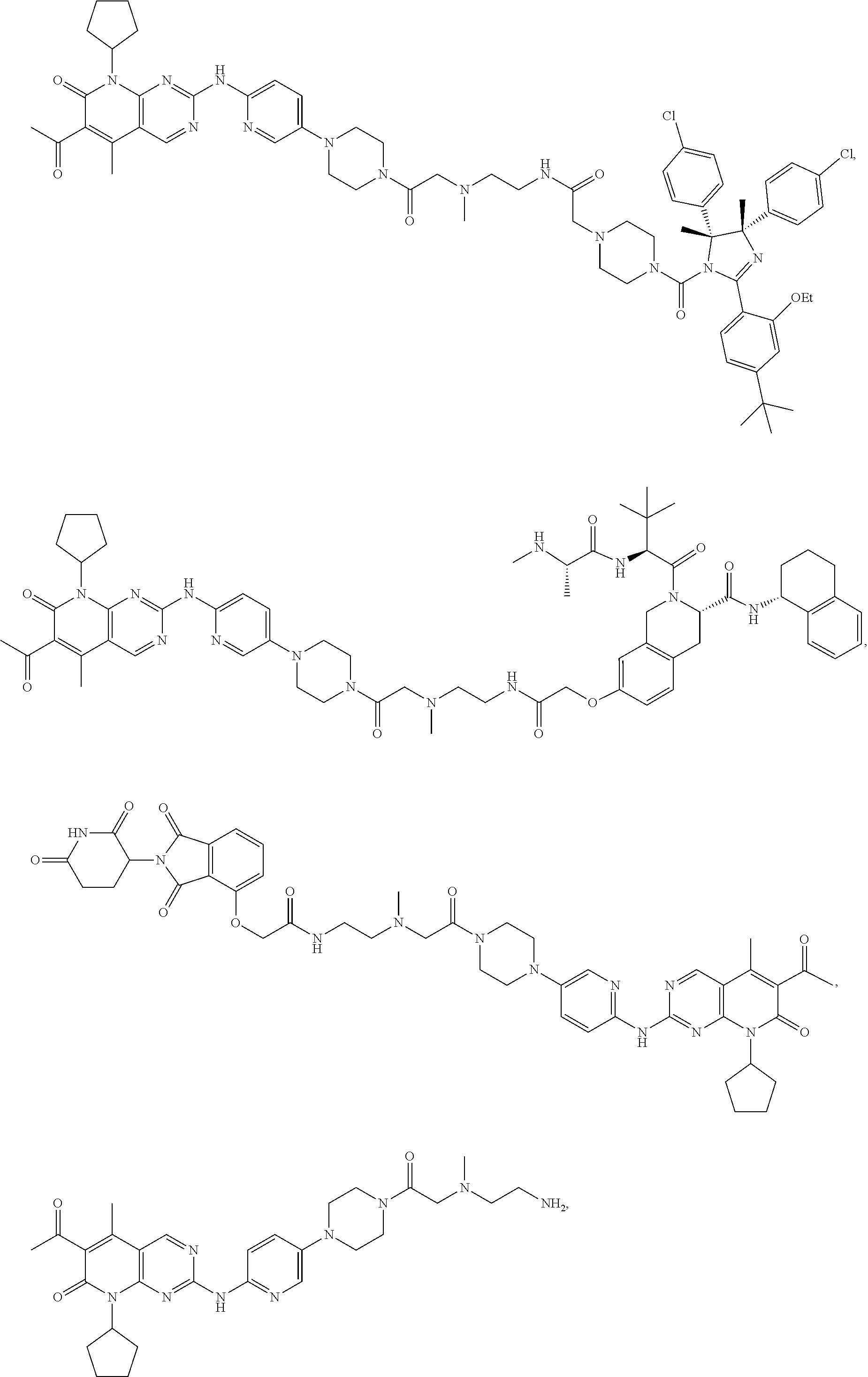 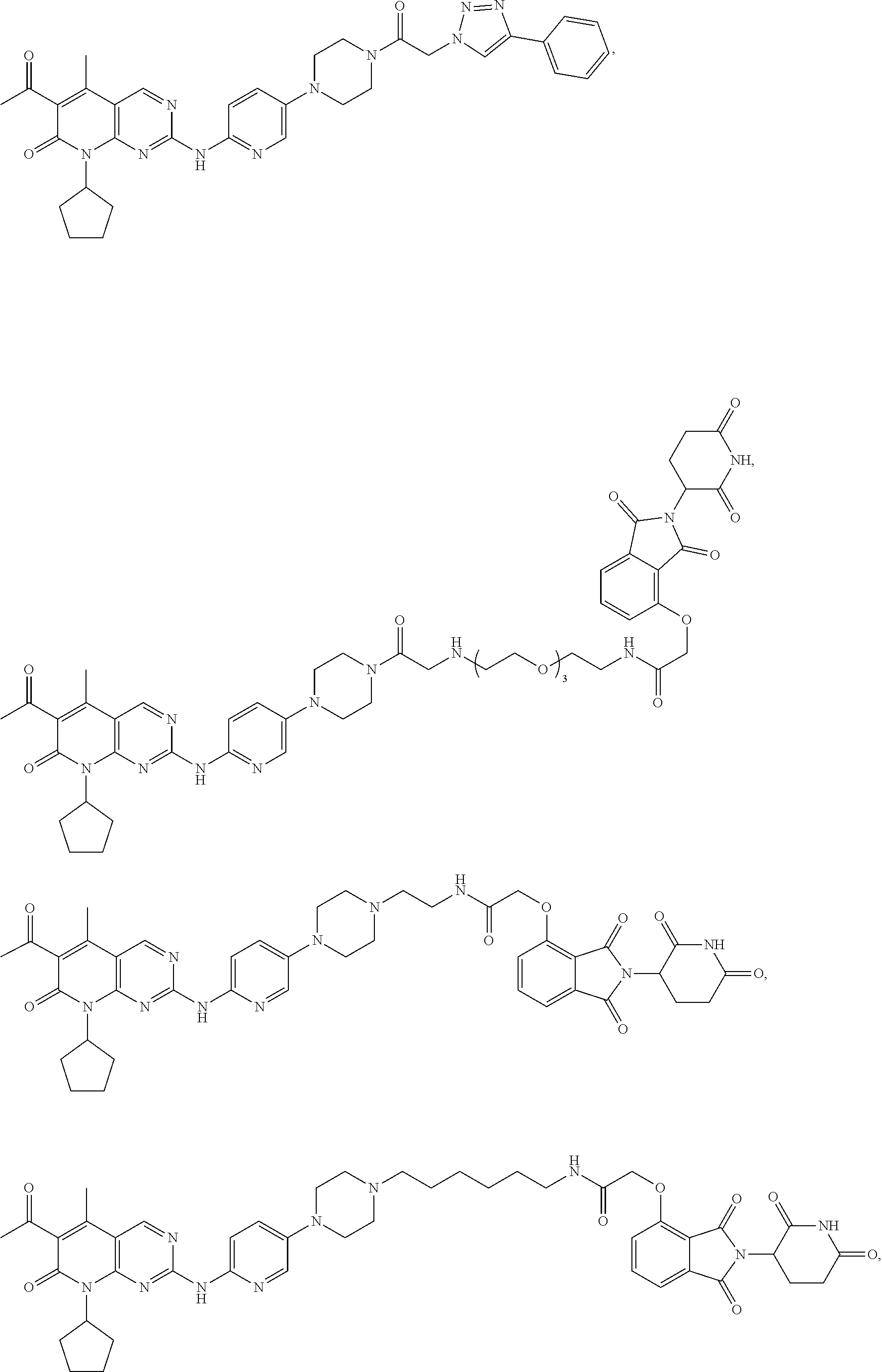 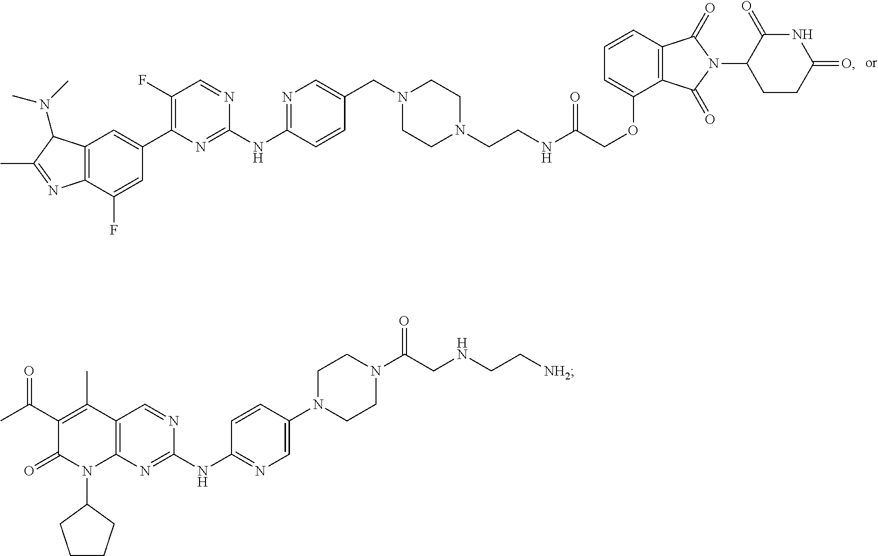 [SMILES: CC1=Nc2c(F)cc(-c3nc(Nc4ccc(CN5CCN(CCNC(=O)COc6cccc7c6C(=O)N(C6CCC(=O)NC6=O)C7=O)CC5)cn4)ncc3F)cc2C1N(C)C ; CC(=O)c1c(C)c2cnc(Nc3ccc(N4CCN(C(=O)CNCCN)CC4)cn3)nc2n(C2CCCC2)c1=O]
or a pharmaceutically acceptable salt thereof.

74. The compound of claim 73, wherein the compound is: 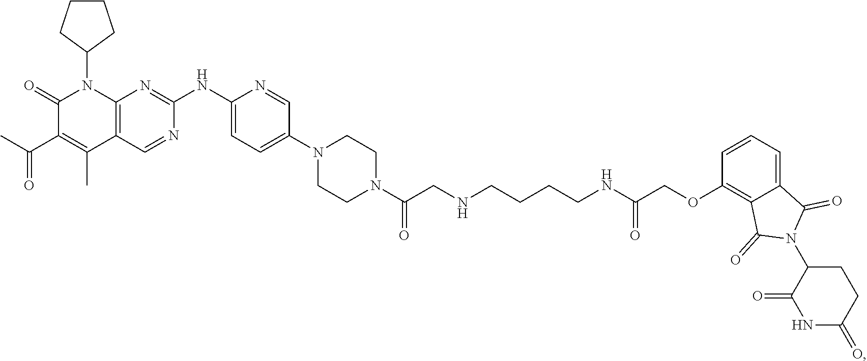 [SMILES: CC(=O)c1c(C)c2cnc(Nc3ccc(N4CCN(C(=O)CNCCCCNC(=O)COc5cccc6c5C(=O)N(C5CCC(=O)NC5=O)C6=O)CC4)cn3)nc2n(C2CCCC2)c1=O]
or a pharmaceutically acceptable salt thereof.

75. A pharmaceutical composition comprising:
(A) a compound according to any one of claims 1-74; and
(B) an excipient.

76. The pharmaceutical composition of claim 75, wherein the composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

77. The pharmaceutical composition of either claim 75 or claim 76, wherein the composition is formulated as a unit dose.

78. A method of treating a disease or disorder in a patient comprising administering a therapeutically effective amount of a compound or composition according to any one of claims 1-77 to the patient.

79. The method of claim 78, wherein the disease or disorder is cancer.

80. The method of claim 79, wherein the cancer has aberrant signaling of CDK4 or CDK6.

81. The method of either claim 79 or claim 80, wherein the cancer is a leukemia, breast cancer, gastric cancer, pancreatic cancer, or liver cancer.

82. The method of claim 81, wherein the leukemia is acute lymphoblastic leukemia, acute myeloid leukemia, or chronic myeloid leukemia.

83. The method according to any one of claims 79-82, wherein the method further comprises administering a second anti-cancer therapy.

84. The method of claim 83, wherein the patient is a mammal.

85. The method of claim 84, wherein the mammal is a human.
